(19) United States
(12) Patent Application Publication
(10) Pub. No.: US 2024/0023433 A1
(43) Pub. Date: Jan. 18, 2024

(54) HETEROCYCLIC COMPOUND, ORGANIC LIGHT-EMITTING DEVICE COMPRISING THE SAME, MANUFACTURING METHOD OF THE SAME AND COMPOSITION FOR ORGANIC LAYER OF ORGANIC LIGHT-EMITTING DEVICE

(71) Applicant: LT MATERIALS CO., LTD., Yongin-si, Gyeonggi-do (KR)

(72) Inventors: Jun Tae MO, Yongin-si (KR); Sol LEE, Yongin-si (KR); Woo Jeong CHAE, Yongin-si (KR); Dong Jun KIM, Yongin-si (KR)

(73) Assignee: LT MATERIALS CO., LTD., Yongin-si, Gyeonggi-do (KR)

(21) Appl. No.: 18/038,602

(22) PCT Filed: Nov. 4, 2021
(86) PCT No.: PCT/KR2021/015941

§ 371 Date: May 24, 2023

(30) Foreign Application Priority Data
Dec. 16, 2020 (KR) 10-2020-0176741

(51) Int. Cl. H10K 85/60; C07D 403/04; C07D 491/048; C07D 495/04; C07D 409/14; C07D 471/04; C07D 403/14; C07D 405/14; C07D 471/20; C07D 519/00; C07D 403/10

(52) U.S. Cl. CPC H10K 85/6572; C07D 403/04; C07D 491/048; C07D 495/04; C07D 409/14; C07D 471/04; C07D 403/14; C07D 405/14; C07D 471/20; C07D 519/00; C07D 403/10; H10K 85/654; H10K 85/657; H10K 85/615; H10K 85/6574; H10K 85/6576; H10K 50/11

(57) ABSTRACT

The present specification relates to a heterocyclic compound represented by Formula 1, an organic light-emitting device comprising the same, a manufacturing method thereof, and a composition for an organic layer thereof.

### TECHNICAL FIELD

This application claims the benefit of priority based on Korean Patent Application No. 10-2020-0176741 filed on Dec. 16, 2020, the entire contents of which are incorporated herein as part of the present specification.

The present invention relates to a heterocyclic compound, an organic light-emitting device comprising the same, a manufacturing method thereof, and a composition for an organic layer thereof.

### BACKGROUND ART

An organic light-emitting device is a kind of self-emitting display device, and has the advantages that the viewing angle is wide, the contrast is excellent, and the response speed is fast.

The organic light-emitting device has a structure in which an organic thin film is disposed between two electrodes. When a voltage is applied to the organic light-emitting device having such a structure, electrons and holes injected from the two electrodes combine in the organic thin film to form a pair, and then emit light while disappearing. The organic thin film may be composed of a single layer or multiple layers, if necessary.

The material for the organic thin film may have a light-emitting function, if necessary. For example, as a material for the organic thin film, a compound capable of constituting the light-emitting layer by itself may be used, or a compound capable of serving as a host or dopant of the host-dopant-based light-emitting layer may be used. In addition, as a material for the organic thin film, a compound capable of performing the roles of hole injection, hole transport, electron blocking, hole blocking, electron transport, electron injection, and the like may be used.

In order to improve the performance, lifespan, or efficiency of the organic light-emitting device, there is a continuous demand for the development of materials for the organic thin film.

### PRIOR ART DOCUMENT

### Patent Document

(Patent Document 1) U.S. Pat. No. 4,356,429

### DISCLOSURE

### Technical Problem

It is an object of the present invention to provide a heterocyclic compound, an organic light-emitting device comprising the same, a manufacturing method thereof, and a composition for an organic layer thereof.

### Technical Solution

The present invention provides a heterocyclic compound represented by following Formula 1:

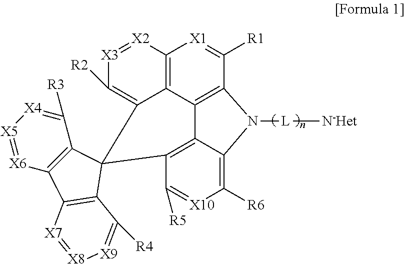

wherein,
X1 to X10 are the same as or different from each other and are each independently N or CRa,
Ra and R1 to R6 are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group; a substituted or unsubstituted C6 to C60 aryl group; or a substituted or unsubstituted C2 to C60 heteroaryl group,
L is a single bond; a substituted or unsubstituted C6 to C60 arylene group; or a substituted or unsubstituted C2 to C60 heteroarylene group,
n is an integer from 0 to 5, with the proviso that when n is 2 or more, L is the same as or different from each other,
N-Het is a substituted or unsubstituted, C2 to C60 monocyclic or polycyclic heterocyclic group containing one or more N.

In addition, the present invention provides an organic light-emitting device comprising a first electrode; a second electrode provided to face the first electrode; and one or more organic layers provided between the first electrode and the second electrode, and wherein one or more of the organic layers comprises the heterocyclic compound represented by Formula 1.

In addition, the present invention provides a composition for an organic layer of the organic light-emitting device comprising the heterocyclic compound represented by Formula 1.

In addition, the present invention provides a method of manufacturing an organic light-emitting device, comprising the steps of: preparing a substrate; forming a first electrode on the substrate; forming one or more organic layers on the first electrode; and forming a second electrode on the organic layer, and wherein the step of forming the organic layers comprises a step of forming one or more organic layers using the composition for an organic layer of the organic light-emitting device.

### Advantageous Effects

The compounds described in the present specification may be used as a material for an organic layer of an organic light-emitting device. The compound may serve as a material for a hole injection layer, a material for a hole transport layer, a material for a light-emitting layer, a material for an electron transport layer, a material for an electron injection layer, and the like in an organic light-emitting device. In particular, the compound may be used as a material for a light-emitting layer of an organic light-emitting device.

Specifically, the compound may be used alone as a light-emitting material, and may be used as a host material or a dopant material of the light-emitting layer. When the compound represented by Formula 1 is used for the organic layer, it is possible to lower the driving voltage, improve the luminous efficiency, and improve the lifespan properties, of the organic light-emitting device.

In particular, the HOMO orbital in the heterocyclic compound represented by Formula 1 of the present invention is delocalized to improve hole mobility, thereby reducing driving voltage and improving efficiency of the organic light-emitting device.

In addition, the heterocyclic compound represented by Formula 1 of the present invention has a high triplet energy level (T₁ level), thereby preventing retrograde energy transfer from the dopant to the host, and exhibiting an effect of well preserving triplet excitons in the light-emitting layer.

In addition, the heterocyclic compound represented by Formula 1 of the present invention facilitates intramolecular charge transfer and reduces the energy gap between the singlet energy level (S₁) and the triplet energy level (T₁), thereby exhibiting an effect of well preserving excitons.

### DESCRIPTION OF DRAWINGS

FIGS. 1 to 3 are views schematically showing a stacked structure of an organic light-emitting device according to one embodiment of the present invention, respectively.

### BEST MODE

Hereinafter, the present application will be described in detail.

In the present specification, the term “substituted” means that a hydrogen atom bonded to a carbon atom of a compound is replaced with another substituent, and the position to be substituted is not limited as long as it is the position at which a hydrogen atom is substituted, that is, the position at which it may be substituted with the substituent. When substituted with two or more substituents, the two or more substituents may be the same as or different from each other.

In the present specification, the term “substituted or unsubstituted” means that it is unsubstituted or substituted with one or more substituents selected from the group consisting of deuterium; halogen; cyano; C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or that it is unsubstituted or substituted with a substituent in which two or more substituents selected from the above-exemplified substituents are connected to each other.

In the present specification, the halogen may be fluorine, chlorine, bromine, or iodine.

In the present specification, the alkyl group includes a linear or branched chain having 1 to 60 carbon atoms, and may be further substituted with another substituent. The number of carbon atoms in the alkyl group may be 1 to 60, specifically 1 to 40, more specifically 1 to 20. Specific examples include, but are not limited to, a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like.

In the present specification, the alkenyl group includes a linear or branched chain having 2 to 60 carbon atoms, and may be further substituted with another substituent. The number of carbon atoms in the alkenyl group may be 2 to 60, specifically 2 to 40, more specifically 2 to 20. Specific examples include, but are not limited to, a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like.

In the present specification, the alkynyl group includes a linear or branched chain having 2 to 60 carbon atoms, and may be further substituted with another substituent. The number of carbon atoms in the alkynyl group may be 2 to 60, specifically 2 to 40, more specifically 2 to 20.

In the present specification, the alkoxy group may be a linear chain, a branched chain or a cyclic chain. Although the number of carbon atoms in the alkoxy group is not particularly limited, it is preferable that the number of carbon atoms is 1-20.

Specifically, it may include, but is not limited to, methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like.

In the present specification, the cycloalkyl group includes a monocyclic or polycyclic ring having 3 to 60 carbon atoms, and may be further substituted with another substituent. In this case, the polycyclic ring refers to a group in which a cycloalkyl group is directly connected or condensed with another cyclic group. In this case, the another cyclic group may be a cycloalkyl group, but may be a different type of cyclic group, for example, a heterocycloalkyl group, an aryl group, a heteroaryl group, or the like. The number of carbon atoms in the cycloalkyl group may be 3 to 60, specifically 3 to 40, more specifically 5 to 20. Specifically, it includes, but is not limited to, a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, 4-tert-butylcyclohexyl group, a cycloheptyl group, a cyclooctyl group, and the like.

In the present invention, the heterocycloalkyl group includes a monocyclic or polycyclic ring containing O, S, Se, N or Si as a heteroatom and having 2 to 60 carbon atoms, and may be further substituted with another substituent. In this case, the polycyclic group refers to a group in which a heterocycloalkyl group is directly connected or condensed with another cyclic group. In this case, the another cyclic group may be a heterocycloalkyl group, but may be a different type of cyclic group, for example, a cycloalkyl group, an aryl group, a heteroaryl group, or the like. The number of carbon atoms in the heterocycloalkyl group may be 2 to 60, specifically 2 to 40, more specifically 3 to 20.

In the present specification, the aryl group includes a monocyclic or polycyclic ring having 6 to 60 carbon atoms, and may be further substituted with another substituent. In this case, the polycyclic group refers to a group in which an aryl group is directly connected or condensed with another cyclic group. In this case, the another cyclic group may be an aryl group, but may be a different type of cyclic group, for example, a cycloalkyl group, a heterocycloalkyl group, a heteroaryl group, or the like. The aryl group includes a spiro group. The number of carbon atoms in the aryl group may be 6 to 60, specifically 6 to 40, more specifically 6 to 25. Specific examples of the aryl group may include, but are not limited to, a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, tetracenyl group, a pentacenyl group, a fluorenyl group, an indenyl group, an acenaphthylenyl group, a benzofluorenyl group, a spirobifluorenyl group, a 2,3-dihydro-1H-indenyl group, condensed cyclic groups thereof, and the like.

In the present specification, the phosphine oxide group is represented by —P(═O) R₁₀₁R₁₀₂, wherein R₁₀₁ and R₁₀₂ are the same as or different from each other, and may each independently be a substituent consisting of at least one of hydrogen; deuterium; a halogen group; an alkyl group; an alkenyl group; an alkoxy group; a cycloalkyl group; an aryl group; and a heterocyclic group. Specifically, it may be substituted with an aryl group, wherein the aryl group may be as exemplified above. For example, the phosphine oxide group includes, but is not limited to, a diphenyl phosphine oxide group, a dinaphthyl phosphine oxide group, and the like.

In the present specification, the silyl group is a substituent containing Si, in which the Si atom is directly connected as a radical, and is represented by —SiR₁₀₄R₁₀₅R₁₀₆, wherein R₁₀₄ to R₁₀₆ are the same as or different from each other, and may each independently be a substituent consisting of at least one of hydrogen; deuterium; a halogen group; an alkyl group; an alkenyl group; an alkoxy group; a cycloalkyl group; an aryl group; and a heterocyclic group. Specific examples of the silyl group include, but is not limited to, a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like.

In the present specification, the fluorenyl group may be substituted, and adjacent substituents may be bonded to each other to form a ring.

When the fluorenyl group is substituted, it may be, but is not limited to,

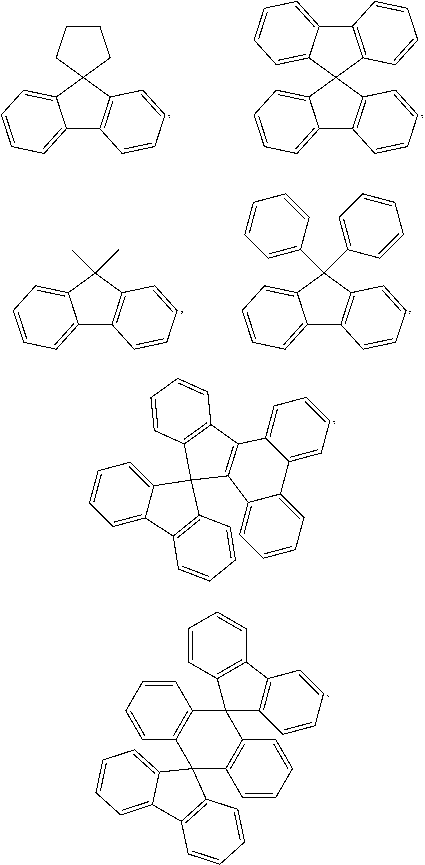

or the like.

In the present specification, the heteroaryl group includes a monocyclic or polycyclic ring containing S, O, Se, N, or Si as a heteroatom and having 2 to 60 carbon atoms, and may be further substituted with another substituent. In this case, the polycyclic group refers to a group in which a heteroaryl group is directly connected or condensed with another cyclic group. In this case, the another cyclic group may be a heteroaryl group, but may be a different type of cyclic group, for example, a cycloalkyl group, a heterocycloalkyl group, an aryl group, or the like. The number of carbon atoms in the heteroaryl group may be 2 to 60, specifically 2 to 40, more specifically 3 to 25. Specific examples of the heteroaryl group may include, but are not limited to, a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolylyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilol group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group; and the number of carbon atoms is not particularly limited, but is preferably 1 to 30. Specific examples of the amine group include, but are not limited to, a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like.

In the present specification, the arylene group refers to a group having two bonding positions on the aryl group, that is, a divalent group. The above description of the aryl group may be applied, except that each of them is a divalent group. In addition, the heteroarylene group refers to a group having two bonding positions on the heteroaryl group, that is, a divalent group. The above description of the heteroaryl group may be applied, except that each of them is a divalent group.

In the present specification, an “adjacent” group may refer to a substituent substituted on an atom directly connected to the atom on which that substituent is substituted, a substituent which is sterically closest to that substituent, or a substituent substituted on the atom on which that substituent is substituted.

For example, two substituents substituted at an ortho position on a benzene ring and two substituents substituted at the same carbon on an aliphatic ring may be interpreted as “adjacent” groups to each other.

In the present invention, “when a substituent is not indicated in the chemical formula or compound structure” means that a hydrogen atom is bonded to a carbon atom. However, since deuterium (²H) is an isotope of hydrogen, some hydrogen atoms may be deuterium.

In one embodiment of the present invention, “when a substituent is not indicated in the chemical formula or compound structure” may mean that hydrogen or deuterium is present at all positions that may be substituted with a substituent. That is, deuterium is an isotope of hydrogen, and thus, some hydrogen atoms may be deuterium, which is an isotope, and in this case, the content of deuterium may be 0% to 100%.

In one embodiment of the present invention, in the case of “when a substituent is not indicated in the chemical formula or compound structure,” hydrogen and deuterium may be used interchangeably in compounds unless deuterium is explicitly excluded, such as “the content of deuterium is 0%,” “the content of hydrogen is 100%,” and “all substituents are hydrogen”.

In one embodiment of the present invention, deuterium is one of the isotopes of hydrogen and is an element having a deuteron consisting of one proton and one neutron as a nucleus, and may be expressed as hydrogen-2, and its element symbol may also be written as D or 2H.

In one embodiment of the present invention, an isotope refers to an atom having the same atomic number (Z) but a different mass number (A), and may also be interpreted as an element having the same number of protons but a different number of neutrons.

In one embodiment of the present invention, the meaning of the T % content of a specific substituent may be defined as the following equation: T2/T1×100 T %, wherein T1 is defined as the total number of substituents that the basic compound can have and T2 is defined as the number of specific substituents substituted among them.

That is, in one example, the 20% content of deuterium in the phenyl group represented by

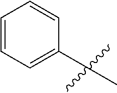 [SMILES: *c1ccccc1]

may mean that the total number of substituents that the phenyl group is 5 (T1 in the equation) and the number of deuterium among them is 1 (T2 in the equation). That is, the 20% content of deuterium in the phenyl group may be represented by the following structural formulas:

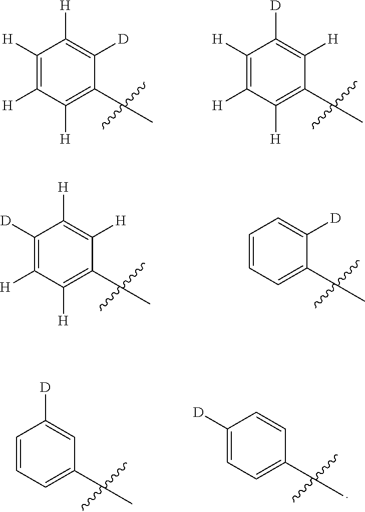 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present invention, the case of “a phenyl group having a deuterium content of 0%” may mean a phenyl group that does not contain deuterium atoms, that is, a phenyl group having 5 hydrogen atoms.

In the present invention, the content of deuterium in the heterocyclic compound represented by Formula 1 may be 0 to 100%, more preferably 30 to 100%.

In the present invention, C6 to C60 aromatic hydrocarbon ring refers to a compound including an aromatic ring consisting of C6 to C60 carbons and hydrogens, for example, includes, but is not limited to, benzene, biphenyl, terphenyl, triphenylene, naphthalene, anthracene, phenalene, phenanthrene, fluorene, pyrene, chrysene, perylene, azulene, and the like, and includes all of the aromatic hydrocarbon ring compounds known in the art as those satisfying the carbon number described above.

The present invention provides a heterocyclic compound represented by following Formula 1:

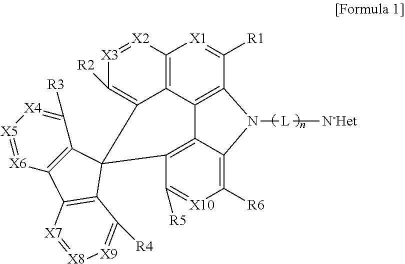

wherein,
X1 to X10 are the same as or different from each other and are each independently N or CRa,
Ra and R1 to R6 are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group; a substituted or unsubstituted C6 to C60 aryl group; or a substituted or unsubstituted C2 to C60 heteroaryl group,
L is a single bond; a substituted or unsubstituted C6 to C60 arylene group; or a substituted or unsubstituted C2 to C60 heteroarylene group,
n is an integer from 0 to 5, with the proviso that when n is 2 or more, L is the same as or different from each other,
N-Het is a substituted or unsubstituted and is C2 to C60 monocyclic or polycyclic heterocyclic group containing one or more N.

As the aliphatic or aromatic hydrocarbon ring or heterocycle that the adjacent groups may form, the structures exemplified by the above-described cycloalkyl group, cycloheteroalkyl group, aryl group and heteroaryl group may be applied, except for those that are not monovalent groups.

In another embodiment of the present invention, the ‘substitution’ of X1 to X10, Ra, R1 to R6, L, and N-Het above may be each independently made with one or more substituents selected from the group consisting of a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In another embodiment of the present invention, the ‘substitution’ of X1 to X10, Ra, R1 to R6, L, and N-Het above may be each independently made with one or more substituents selected from the group consisting of a C1 to C10 alkyl group; a C6 to C30 aryl group; and a C2 to C30 heteroaryl group.

In another embodiment of the present invention, the substitution of X1 to X10, Ra, R1 to R6, L, and N-Het above may be each independently made with one or more substituents selected from the group consisting of a C1 to C5 alkyl group; a C6 to C20 aryl group; and a C2 to C20 heteroaryl group.

In another embodiment of the present invention, the ‘substitution’ of X1 to X10, Ra, R1 to R6, L, and N-Het above may be each independently made with one or more substituents selected from the group consisting of a methyl group, an ethyl group, a linear or branched propyl group, a linear or branched butyl group, a linear or branched pentyl group, a phenyl group, a naphthalenyl group, a pyridinyl group, an anthracenyl group, a carbazolyl group, a dibenzothiophenyl group, a dibenzofuranyl group, and a phenanthrenyl group.

In another embodiment of the present invention, the substitution of X1 to X10, Ra, R1 to R6, L, and N-Het above may be each independently made with one or more substituents selected from the group consisting of a methyl group, an ethyl group, a linear or branched propyl group, a linear or branched butyl group, and a linear or branched pentyl group.

In one embodiment of the present invention, Formula 1 above may be represented by following Formulas 1-1 to 1-3:

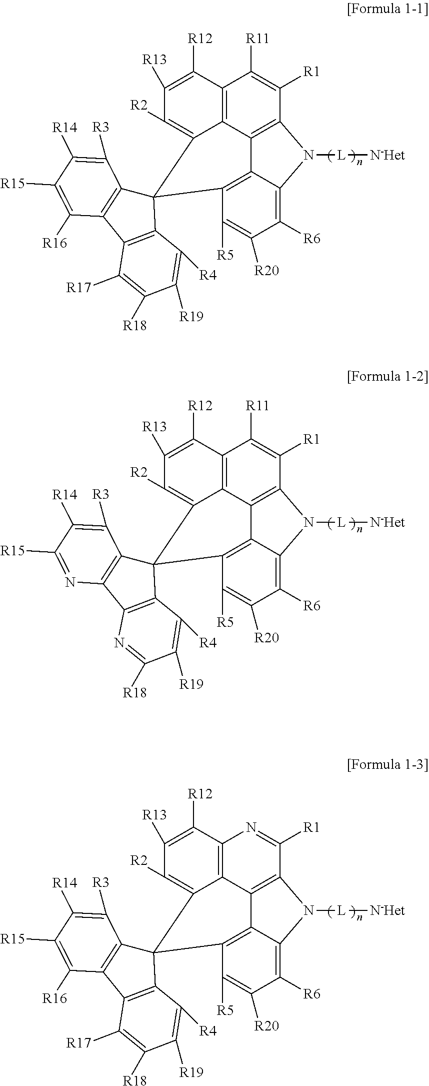

wherein,
R11 to R20 are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group, a substituted or unsubstituted C6 to C60 aryl group, or a substituted or unsubstituted C2 to C60 heteroaryl group,
R1 to R6, L, n, and N-Het are the same as defined in Formula 1.

In one embodiment of the present invention, R1 to R6 and R11 to R20 in Formula 1-1 above may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, R1 to R6 and R11 to R20 in Formula 1-1 above may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, R1 to R6 and R11 to R20 in Formula 1-1 may be the same as or different from each other and may be independently hydrogen or deuterium.

In another embodiment of the present invention, all of R1 to R6 and R11 to R20 in Formula 1-1 may be hydrogen.

In one embodiment of the present invention, in Formula 1-1 above, any one of R11 to R20 may be halogen; a substituted or unsubstituted C1 to C60 alkyl group; a substituted or unsubstituted C6 to C60 aryl group; or a substituted or unsubstituted C2 to C60 heteroaryl group, and the rest may be hydrogen or deuterium.

In another embodiment of the present invention, in Formula 1-1 above, any one of R11 to R20 may be halogen; a substituted or unsubstituted C1 to C30 alkyl group; a substituted or unsubstituted C6 to C30 aryl group; or a substituted or unsubstituted C2 to C30 heteroaryl group, and the rest may be hydrogen or deuterium.

In another embodiment of the present invention, in Formula 1-1 above, any one of R11 to R20 may be halogen; a substituted or unsubstituted C1 to C20 alkyl group; a substituted or unsubstituted C6 to C20 aryl group; or a substituted or unsubstituted C2 to C20 heteroaryl group, and the rest may be hydrogen or deuterium.

In another embodiment of the present invention, in Formula 1-1 above, any one of R11 to R20 may be a fluoro group; tert-butyl group; a methyl group; a substituted or unsubstituted phenyl group, a naphthyl group, a dibenzofuranyl group, or a carbazolyl group, and the rest may be hydrogen.

In one embodiment of the present invention, in Formula 1-1 above, two of R11 to R20 may be a substituted or unsubstituted C6 to C60 aryl group or a substituted or unsubstituted C2 to C60 heteroaryl group, and the rest may be hydrogen or deuterium.

In another embodiment of the present invention, in Formula 1-1 above, two of R1l to R20 may be a substituted or unsubstituted C6 to C30 aryl group or a substituted or unsubstituted C2 to C30 heteroaryl group, and the rest may be hydrogen or deuterium.

In another embodiment of the present invention, in Formula 1-1 above, two of R1l to R20 may be a substituted or unsubstituted C6 to C20 aryl group or a substituted or unsubstituted C2 to C20 heteroaryl group, and the rest may be hydrogen or deuterium.

In another embodiment of the present invention, in Formula 1-1 above, two of R11 to R20 may be a substituted or unsubstituted C6 to C20 aryl group, and the rest may be hydrogen or deuterium.

In another embodiment of the present invention, in Formula 1-1 above, two of R11 to R20 may be a substituted or unsubstituted phenyl group, and the rest may be hydrogen or deuterium.

In another embodiment of the present invention, in Formula 1-1 above, R11 and R20, or R12 and R20 of R11 to R20 may be a substituted or unsubstituted C6 to C60 aryl group or a substituted or unsubstituted C2 to C60 heteroaryl group, and the rest may be hydrogen or deuterium.

In another embodiment of the present invention, in Formula 1-1 above, R11 and R20, or R12 and R20 of R11 to R20 may be a substituted or unsubstituted C6 to C30 aryl group or a substituted or unsubstituted C2 to C30 heteroaryl group, and the rest may be hydrogen or deuterium.

In another embodiment of the present invention, in Formula 1-1 above, R11 and R20, or R12 and R20 of R11 to R20 may be a substituted or unsubstituted C6 to C20 aryl group or a substituted or unsubstituted C2 to C20 heteroaryl group, and the rest may be hydrogen or deuterium.

In another embodiment of the present invention, in Formula 1-1 above, R11 and R20, or R12 and R20 of R11 to R20 may be a substituted or unsubstituted C6 to C20 aryl group, and the rest may be hydrogen or deuterium.

In another embodiment of the present invention, in Formula 1-1 above, R11 and R20, or R12 and R20 of R11 to R20 may be a substituted or unsubstituted phenyl group, and the rest may be hydrogen or deuterium.

In one embodiment of the present invention, R1 to R6, R11 to R15, and R18 to R20 in Formula 1-2 above may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, R1 to R6, R11 to R15, and R18 to R20 in Formula 1-2 above may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, R1 to R6, R11 to R15, and R18 to R20 in Formula 1-2 above may be the same as or different from each other and may be each independently hydrogen; deuterium; a substituted or unsubstituted C6 to C20 aryl group; or a substituted or unsubstituted C2 to C20 heteroaryl group.

In another embodiment of the present invention, R1 to R6, R11 to R15, and R18 to R20 in Formula 1-2 may be the same as or different from each other and may be hydrogen or deuterium.

In another embodiment of the present invention, all of R1 to R6, R11 to R15, and R18 to R20 in Formula 1-2 may be hydrogen.

In one embodiment of the present invention, R1 to R6 and R12 to R20 in Formula 1-3 above may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, R1 to R6 and R12 to R20 in Formula 1-3 above may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, R1 to R6 and R12 to R20 in Formula 1-3 above may be the same as or different from each other and may be each independently hydrogen; deuterium; a substituted or unsubstituted C6 to C20 aryl group; or a substituted or unsubstituted C2 to C20 heteroaryl group.

In another embodiment of the present invention, R1 to R6 and R12 to R20 in Formula 1-3 above may be the same as or different from each other and may be hydrogen or deuterium.

In another embodiment of the present invention, all of R1 to R6 and R12 to R20 in Formula 1-3 may be hydrogen.

In another embodiment of the present invention, the ‘substitution’ of R11 to R20 above may be each independently made with one or more substituents selected from the group consisting of a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In another embodiment of the present invention, the ‘substitution’ of R11 to R20 above may be each independently made with one or more substituents selected from the group consisting of a C1 to C10 alkyl group; a C6 to C30 aryl group; and a C2 to C30 heteroaryl group.

In another embodiment of the present invention, the ‘substitution’ of R11 to R20 above may be each independently made with one or more substituents selected from the group consisting of a C1 to C5 alkyl group; a C6 to C20 aryl group; and a C2 to C20 heteroaryl group.

In another embodiment of the present invention, the ‘substitution’ of R11 to R20 may be each independently made with one or more substituents selected from the group consisting of a methyl group, an ethyl group, a linear or branched propyl group, a linear or branched butyl group, a linear or branched pentyl group, a phenyl group, a naphthalenyl group, a pyridinyl group, an anthracenyl group, a carbazolyl group, a dibenzothiophenyl group, a dibenzofuranyl group, and a phenanthrenyl group.

In another embodiment of the present invention, the ‘substitution’ of R11 to R20 above may be each independently made with one or more substituents selected from the group consisting of a methyl group, an ethyl group, a linear or branched propyl group, a linear or branched butyl group, and a linear or branched pentyl group.

In one embodiment of the present invention, N-Het is the heterocyclic compound represented by any one of following Formulas 1-4 to 1-8:

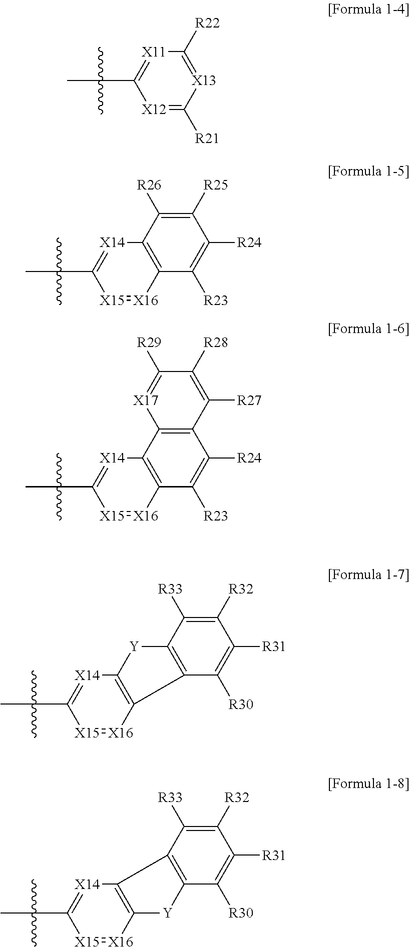

wherein,
X11 to X13 are the same as or different from each other and are each independently N or CRb,
X14 to X16 are the same as or different from each other and are each independently N or CRc,
X17 is N or CRd;
Rb, Rc, and Rd are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 above are the same as or different from each other and are each independently a substituted or unsubstituted C1 to C60 alkyl group; a substituted or unsubstituted C6 to C60 aryl group; or a substituted or unsubstituted C2 to C60 heteroaryl group,
Y is O or S,
R21 to R33 are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group; a substituted or unsubstituted C6 to C60 aryl group; or a substituted or unsubstituted C2 to C60 heteroaryl group.

In one embodiment of the present invention, in Formula 1-4 above, two or more of X11 to X13 may be N, and Rb may be each independently hydrogen; deuterium; a substituted or unsubstituted C6 to C60 aryl group; or a substituted or unsubstituted C2 to C60 heteroaryl group.

In another embodiment of the present invention, in Formula 1-4 above, two or more of X11 to X13 may be N, and Rb may be each independently hydrogen; deuterium; a substituted or unsubstituted C6 to C30 aryl group; or a substituted or unsubstituted C2 to C30 heteroaryl group.

In another embodiment of the present invention, in Formula 1-4 above, two or more of X11 to X13 may be N, and Rb may be each independently hydrogen; deuterium; a substituted or unsubstituted C6 to C20 aryl group; or a substituted or unsubstituted C2 to C20 heteroaryl group.

In another embodiment of the present invention, in Formula 1-4 above, two or more of X11 to X13 may be N, and Rb may be each independently hydrogen; deuterium; a substituted or unsubstituted phenyl group; or a substituted or unsubstituted carbazolyl group.

In another embodiment of the present invention, all of X11 to X13 in Formula 1-4 may be N.

In one embodiment of the present invention, R21 and R22 in Formula 1-4 above may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, R21 and R22 in Formula 1-4 above may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, R21 and R22 in Formula 1-4 above may be the same as or different from each other and may be each independently hydrogen; deuterium; a substituted or unsubstituted C6 to C20 aryl group; or a substituted or unsubstituted C2 to C20 heteroaryl group.

In another embodiment of the present invention, R21 and R22 in Formula 1-4 above may be the same as or different from each other and may be each independently hydrogen; deuterium; a substituted or unsubstituted phenyl group, naphthyl group, or fluorenyl group; or a substituted or unsubstituted dibenzofuranyl group, dibenzothiophenyl group, carbazolyl group or quinolinyl group.

In one embodiment of the present invention, in Formula 1-5 above, two of X14 to X16 may be N, and the rest may be CRc. In addition, Rc may be deuterium; a substituted or unsubstituted C6 to C60 aryl group; or a substituted or unsubstituted C2 to C60 heteroaryl group.

In another embodiment of the present invention, in Formula 1-5 above, two of X14 to X16 may be N, and the rest may be CRc. In addition, Rc may be deuterium; a substituted or unsubstituted C6 to C30 aryl group; or a substituted or unsubstituted C2 to C30 heteroaryl group.

In another embodiment of the present invention, in Formula 1-5 above, two of X14 to X16 may be N, and the rest may be CRc. In addition, Rc may be deuterium; a substituted or unsubstituted C6 to C20 aryl group, or a substituted or unsubstituted C2 to C20 heteroaryl group.

In another embodiment of the present invention, in Formula 1-5 above, two of X14 to X16 may be N, and the rest may be CRc. In addition, Rc may be deuterium; a substituted or unsubstituted phenyl group, naphthyl group, or fluorene group; or a substituted or unsubstituted dibenzofuranyl group, dibenzothiophenyl group, benzonaphthofuranyl group, benzonaphthothiophenyl group, benzocarbazolyl group, or dibenzocarbazolyl group.

In another embodiment of the present invention, in Formula 1-5 above, X14 and X15, or X14 and X16 of X14 to X16 may be N, and the rest may be CRc. In addition, Rc may be deuterium; a substituted or unsubstituted C6 to C60 aryl group; or a substituted or unsubstituted C2 to C60 heteroaryl group.

In another embodiment of the present invention, in Formula 1-5 above, X14 and X15, or X14 and X16 of X14 to X16 may be N, and the rest may be CRc. In addition, Rc may be deuterium; a substituted or unsubstituted C6 to C30 aryl group; or a substituted or unsubstituted C2 to C30 heteroaryl group.

In another embodiment of the present invention, in Formula 1-5 above, X14 and X15, or X14 and X16 of X14 to X16 may be N, and the rest may be CRc. In addition, Rc may be deuterium; a substituted or unsubstituted C6 to C20 aryl group; or a substituted or unsubstituted C2 to C20 heteroaryl group.

In another embodiment of the present invention, in Formula 1-5 above, X14 and X15, or X14 and X16 of X14 to X16 may be N, and the rest may be CRc. In addition, Rc may be deuterium; a substituted or unsubstituted phenyl group, naphthyl group, or fluorene group; or a substituted or unsubstituted dibenzofuranyl group, dibenzothiophenyl group, benzonaphthofuranyl group, benzonaphthothiophenyl group, benzocarbazolyl group, or dibenzocarbazolyl group.

In one embodiment of the present invention, R23 to R26 in Formula 1-5 above may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, R23 to R26 in Formula 1-5 above may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, R23 to R26 in Formula 1-5 above may be the same as or different from each other and may be each independently hydrogen; deuterium; a substituted or unsubstituted C6 to C20 aryl group; or a substituted or unsubstituted C2 to C20 heteroaryl group.

In another embodiment of the present invention, R23 to R26 in Formula 1-5 above may be the same as or different from each other and may be each independently hydrogen; deuterium; or a substituted or unsubstituted C6 to C20 aryl group.

In another embodiment of the present invention, R23 to R26 in Formula 1-5 may be the same as or different from each other and may be hydrogen or deuterium.

In one embodiment of the present invention, in Formula 1-6 above, two of X14 to X16 may be N and the rest may be CRc, and X17 may be CRd, wherein Rc and Rd may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, in Formula 1-6 above, two of X14 to X16 may be N and the rest may be CRc, and X17 may be CRd, wherein Rc and Rd may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, in Formula 1-6 above, two of X14 to X16 may be N and the rest may be CRc, and X17 may be CRd, wherein Rc and Rd may be the same as or different from each other and may be each independently hydrogen; deuterium; a substituted or unsubstituted C6 to C20 aryl group; or a substituted or unsubstituted C2 to C20 heteroaryl group.

In another embodiment of the present invention, in Formula 1-6 above, two of X14 to X16 may be N and the rest may be CRc, and X17 may be CRd, wherein Rc and Rd may be the same as or different from each other and may be each independently hydrogen; deuterium; or a substituted or unsubstituted C6 to C20 aryl group.

In another embodiment of the present invention, in Formula 1-6 above, two of X14 to X16 may be N and the rest may be CRc, and X17 may be CRd, wherein Rc may be a substituted or unsubstituted phenyl group, and Rd may be hydrogen or deuterium.

In another embodiment of the present invention, in Formula 1-6 above, X14 and X15 may be N, X16 may be CRc, and X17 may be CRd. Rc and Rd may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, in Formula 1-6 above, X14 and X15 may be N, X16 may be CRc, and X17 may be CRd. Rc and Rd may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, in Formula 1-6 above, X14 and X15 may be N, X16 may be CRc, and X17 may be CRd. Rc and Rd may be the same as or different from each other and may be each independently hydrogen; deuterium; a substituted or unsubstituted C6 to C20 aryl group; or a substituted or unsubstituted C2 to C20 heteroaryl group.

In another embodiment of the present invention, in Formula 1-6 above, X14 and X15 may be N, X16 may be CRc, and X17 may be CRd. Rc and Rd may be the same as or different from each other and may be each independently hydrogen; deuterium; or a substituted or unsubstituted C6 to C20 aryl group.

In another embodiment of the present invention, in Formula 1-6 above, X14 and X15 may be N, X16 may be CRc, and X17 may be CRd. Rc may be a substituted or unsubstituted phenyl group, and Rd may be hydrogen or deuterium.

In another embodiment of the present invention, in Formula 1-6 above, any one of X14 to X16 may be N and the rest may be CRc, and X17 may be N, wherein Rc may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, in Formula 1-6 above, any one of X14 to X16 may be N and the rest may be CRc, and X17 may be N, wherein Rc may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, in Formula 1-6 above, any one of X14 to X16 may be N and the rest may be CRc, and X17 may be N, wherein Rc may be the same as or different from each other and may be each independently hydrogen; deuterium; a substituted or unsubstituted C6 to C20 aryl group; or a substituted or unsubstituted C2 to C20 heteroaryl group.

In another embodiment of the present invention, in Formula 1-6 above, any one of X14 to X16 may be N and the rest may be CRc, and X17 may be N, wherein Rc may be the same as or different from each other and may be each independently hydrogen; deuterium; or a substituted or unsubstituted C6 to C20 aryl group.

In another embodiment of the present invention, in Formula 1-6 above, any one of X14 to X16 may be N and the rest may be CRc, and X17 may be N, wherein Rc may be the same as or different from each other and may be each independently hydrogen or deuterium.

In another embodiment of the present invention, R23, R24, and R27 to R29 in Formula 1-6 above may be the same as or different from each other and may be each independently hydrogen; deuterium; a substituted or unsubstituted C6 to C60 aryl group; or a substituted or unsubstituted C2 to C60 heteroaryl group.

In another embodiment of the present invention, R23, R24, and R27 to R29 in Formula 1-6 above may be the same as or different from each other and may be each independently hydrogen; deuterium; a substituted or unsubstituted C6 to C30 aryl group; or a substituted or unsubstituted C2 to C30 heteroaryl group.

In another embodiment of the present invention, R23, R24, and R27 to R29 in Formula 1-6 above may be hydrogen; deuterium; a substituted or unsubstituted C6 to C20 aryl group; or a substituted or unsubstituted C2 to C20 heteroaryl group.

In another embodiment of the present invention, R23, R24, and R27 to R29 in Formula 1-6 above may be hydrogen; deuterium; or a substituted or unsubstituted C6 to C20 aryl group.

In one embodiment of the present invention, in Formulas 1-7 and 1-8 above, two of X14 to X16 may be N, and the rest may be CRc, wherein Rc may be hydrogen; deuterium; a substituted or unsubstituted C6 to C60 aryl group, or a substituted or unsubstituted C2 to C60 heteroaryl group.

In another embodiment of the present invention, in Formulas 1-7 and 1-8 above, two of X14 to X16 may be N, and the rest may be CRc, wherein Rc may be hydrogen; deuterium; a substituted or unsubstituted C6 to C30 aryl group; or a substituted or unsubstituted C2 to C30 heteroaryl group.

In another embodiment of the present invention, in Formulas 1-7 and 1-8 above, two of X14 to X16 may be N, and the rest may be CRc, wherein Rc may be hydrogen; deuterium; a substituted or unsubstituted C6 to C20 aryl group; or a substituted or unsubstituted C2 to C20 heteroaryl group.

In another embodiment of the present invention, in Formulas 1-7 and 1-8 above, two of X14 to X16 may be N, and the rest may be CRc, wherein Rc may be deuterium; a substituted or unsubstituted phenyl group, naphthyl group, or fluorene group; or a substituted or unsubstituted dibenzofuranyl group, dibenzothiophenyl group, benzonaphthofuranyl group, benzonaphthothiophenyl group, benzocarbazolyl group, or dibenzocarbazolyl group.

In another embodiment of the present invention, in Formulas 1-7 and 1-8 above, X14 and X15, or X14 and X16 of X14 to X16 may be N, and the rest may be CRc, wherein Rc may be hydrogen; deuterium; a substituted or unsubstituted C6 to C60 aryl group; or a substituted or unsubstituted C2 to C60 heteroaryl group.

In another embodiment of the present invention, in Formulas 1-7 and 1-8 above, X14 and X15, or X14 and X16 of X14 to X16 may be N, and the rest may be CRc, wherein Rc may be hydrogen; deuterium; a substituted or unsubstituted C6 to C30 aryl group; or a substituted or unsubstituted C2 to C30 heteroaryl group.

In another embodiment of the present invention, in Formulas 1-7 and 1-8 above, X14 and X15, or X14 and X16 of X14 to X16 may be N, and the rest may be CRc, wherein Rc may be hydrogen, deuterium, a substituted or unsubstituted C6 to C20 aryl group, or a substituted or unsubstituted C2 to C20 heteroaryl group.

In another embodiment of the present invention, in Formulas 1-7 and 1-8 above, X14 and X15, or X14 and X16 of X14 to X16 may be N, and the rest may be CRc, wherein Rc may be deuterium; a substituted or unsubstituted phenyl group, naphthyl group, or fluorenyl group; or a substituted or unsubstituted dibenzofuranyl group, dibenzothiophenyl group, benzonaphthofuranyl group, benzonaphthothiophenyl group, benzocarbazolyl group, or dibenzocarbazolyl group.

In another embodiment of the present invention, in Formulas 1-7 and 1-8 above, R30 to R33 may be the same as or different from each other and may be each independently hydrogen; deuterium; a substituted or unsubstituted C6 to C60 aryl group; or a substituted or unsubstituted C2 to C60 heteroaryl group; or two or more groups adjacent to each other may combine with each other to form a substituted or unsubstituted C6 to C60 aromatic hydrocarbon ring or a substituted or unsubstituted C2 to C60 heterocycle.

In another embodiment of the present invention, in Formulas 1-7 and 1-8 above, R30 to R33 may be the same as or different from each other and may be each independently hydrogen; deuterium; a substituted or unsubstituted C6 to C30 aryl group; or a substituted or unsubstituted C2 to C30 heteroaryl group; or two or more groups adjacent to each other may combine with each other to form a substituted or unsubstituted C6 to C30 aromatic hydrocarbon ring or a substituted or unsubstituted C2 to C30 heterocycle.

In another embodiment of the present invention, in Formulas 1-7 and 1-8 above, R30 to R33 may be the same as or different from each other and may be each independently hydrogen; deuterium; a substituted or unsubstituted C6 to C20 aryl group; or a substituted or unsubstituted C2 to C20 heteroaryl group, or two or more groups adjacent to each other may combine with each other to form a substituted or unsubstituted C6 to C30 aromatic hydrocarbon ring or a substituted or unsubstituted C2 to C20 heterocycle.

In another embodiment of the present invention, in Formulas 1-7 and 1-8 above, R30 to R33 may be the same as or different from each other and may be each independently hydrogen or deuterium, or two or more groups adjacent to each other may combine with each other to form a substituted or unsubstituted benzene ring.

In another embodiment of the present invention, the ‘substitution’ of X11 to X17, R21 to R33, Rb, Rc, and Rd may be each independently made with one or more substituents selected from the group consisting of a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In another embodiment of the present invention, the ‘substitution’ of X11 to X17, R21 to R33, Rb, Rc, and Rd may be each independently made with one or more substituents selected from the group consisting of a C1 to C10 alkyl group; a C6 to C30 aryl group; and a C2 to C30 heteroaryl group.

In another embodiment of the present invention, the ‘substitution’ of X11 to X17, R21 to R33, Rb, Rc, and Rd may be each independently made with one or more substituents selected from the group consisting of a C1 to C5 alkyl group; a C6 to C20 aryl group; and a C2 to C20 heteroaryl group.

In another embodiment of the present invention, the ‘substitution’ of X11 to X17, R21 to R33, Rb, Rc, and Rd may be each independently made with one or more substituents selected from the group consisting of a methyl group, an ethyl group, a linear or branched propyl group, a linear or branched butyl group, a linear or branched pentyl group, a phenyl group, a naphthalenyl group, a pyridinyl group, an anthracenyl group, a carbazolyl group, a dibenzothiophenyl group, a dibenzofuranyl group, and a phenanthrenyl group.

In another embodiment of the present invention, the ‘substitution’ of X11 to X17, R21 to R33, Rb, Rc, and Rd may be each independently made with a methyl group, an ethyl group, a linear or branched propyl group, a linear or branched butyl group, and a linear or branched pentyl group.

In one embodiment of the present invention, Formula 1 above is the heterocyclic compound represented by any one of following compounds:

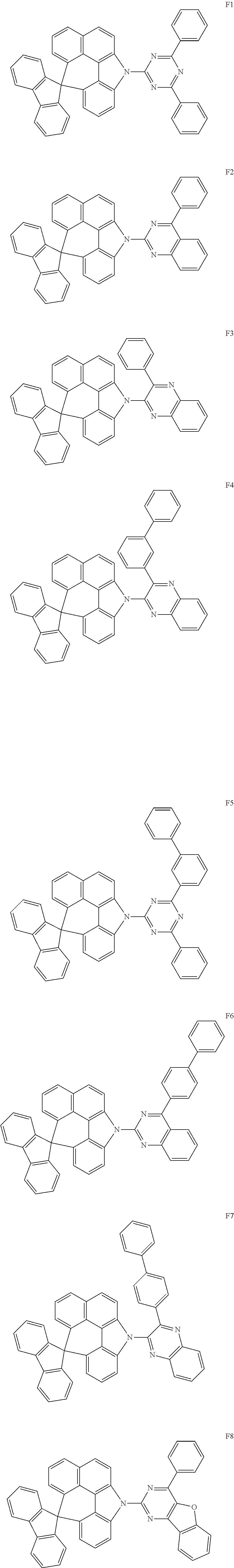

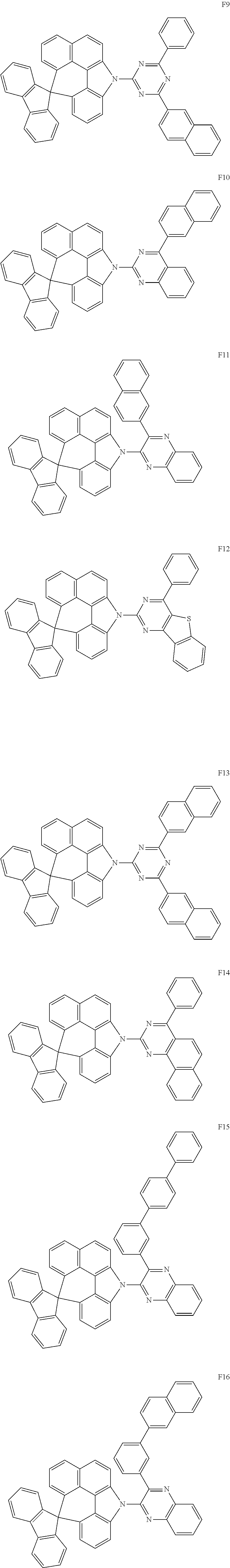

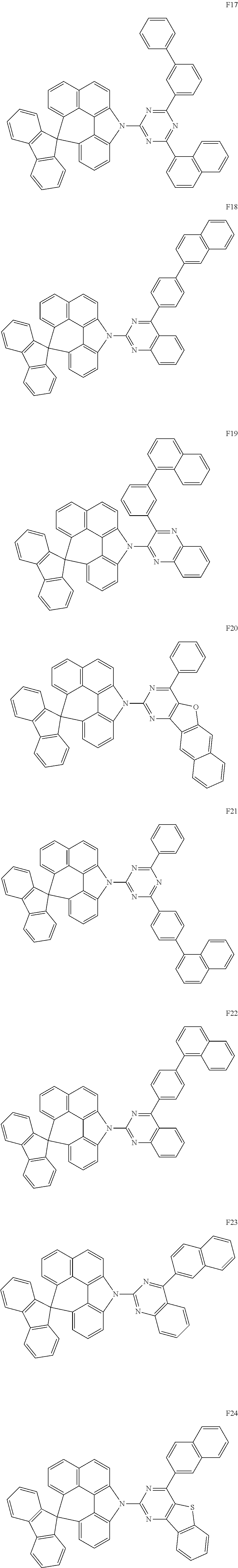

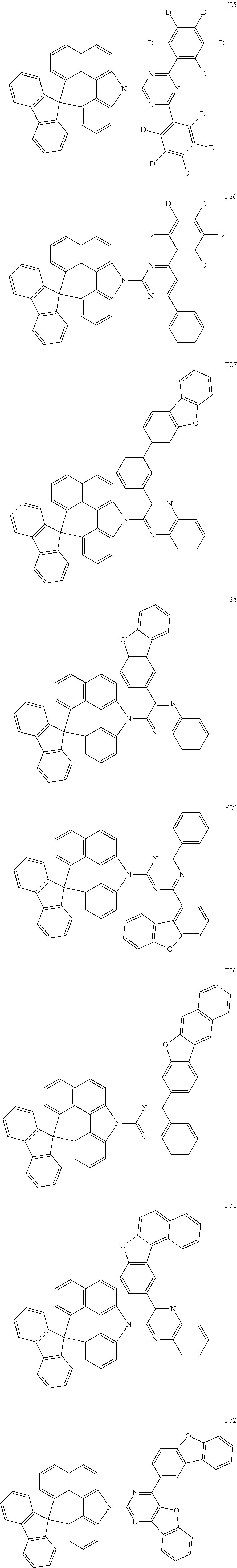

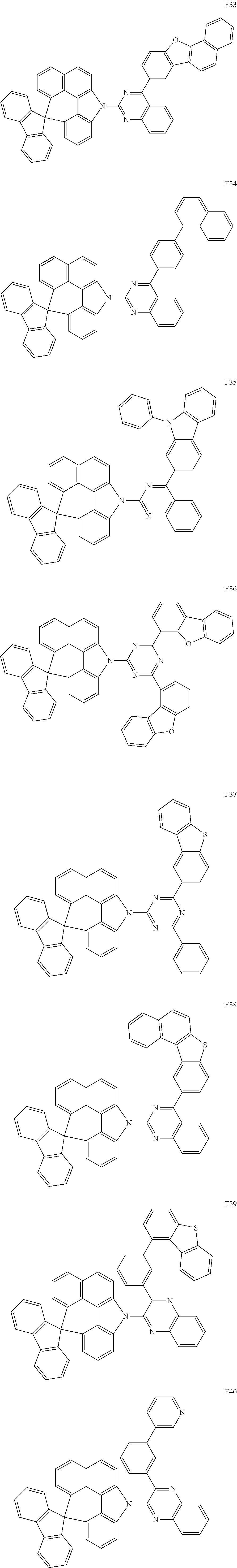

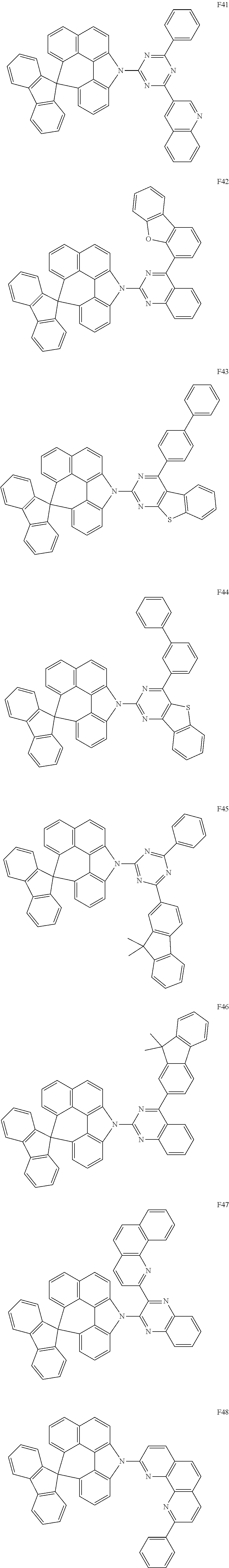

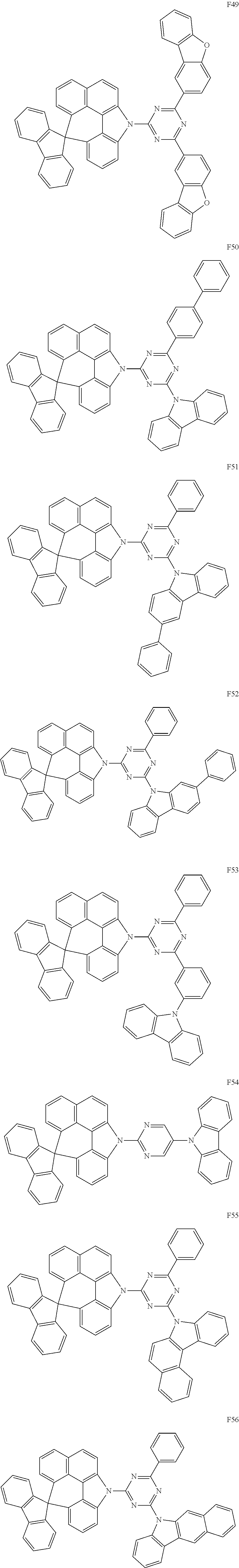

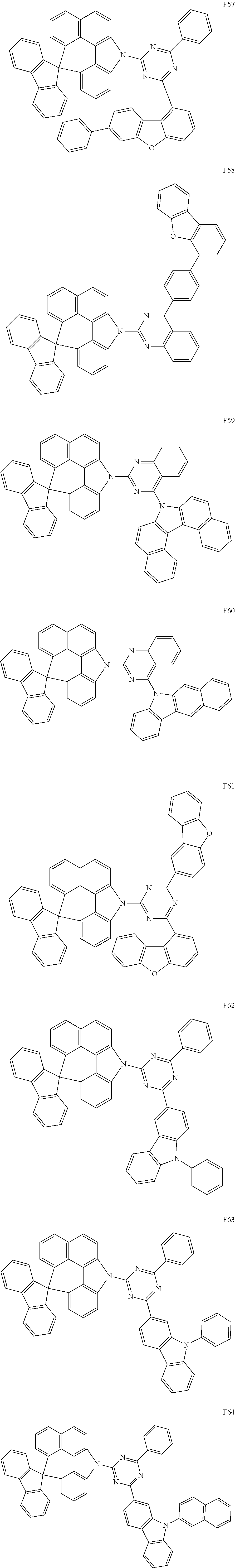

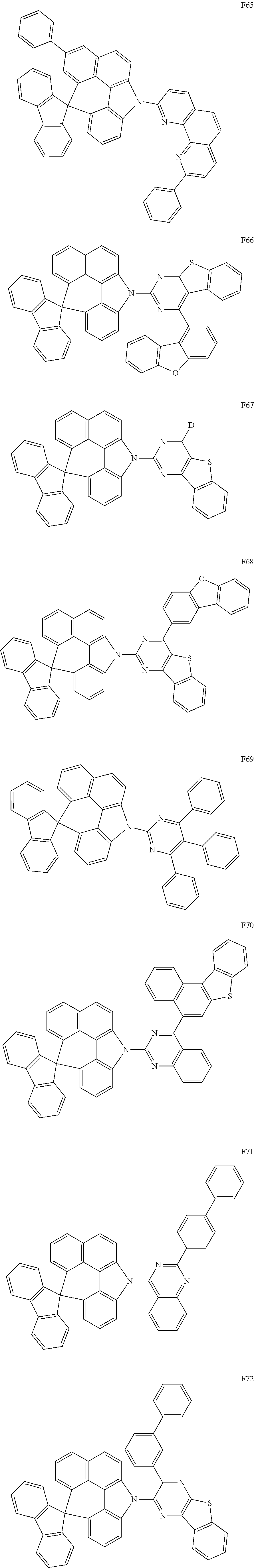

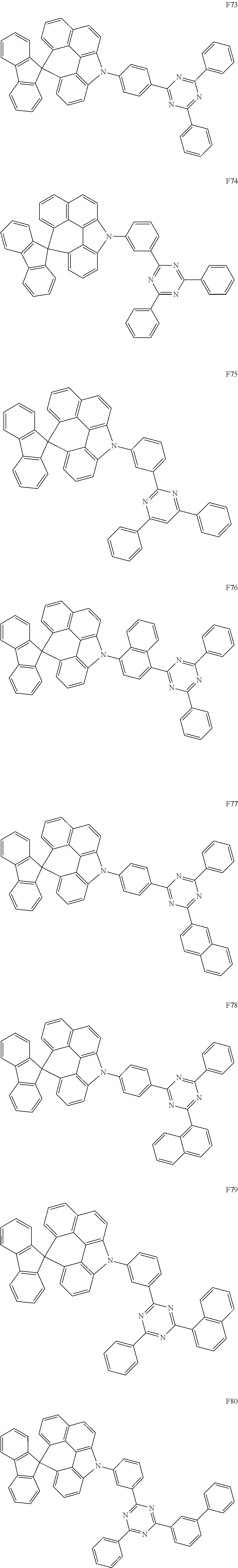

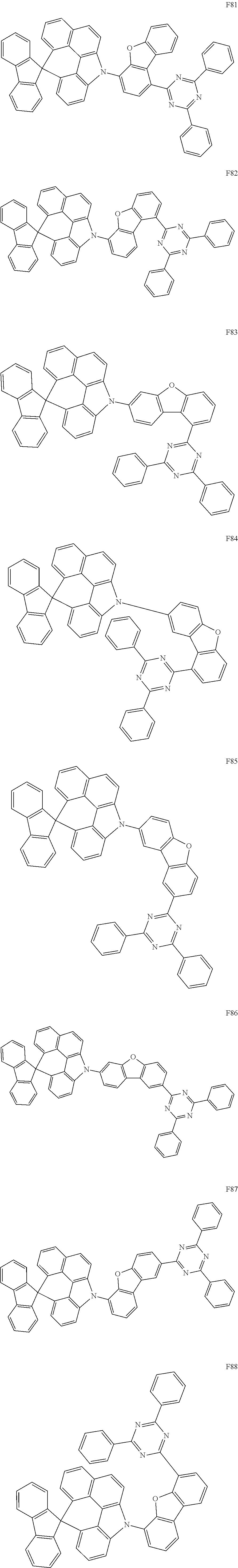

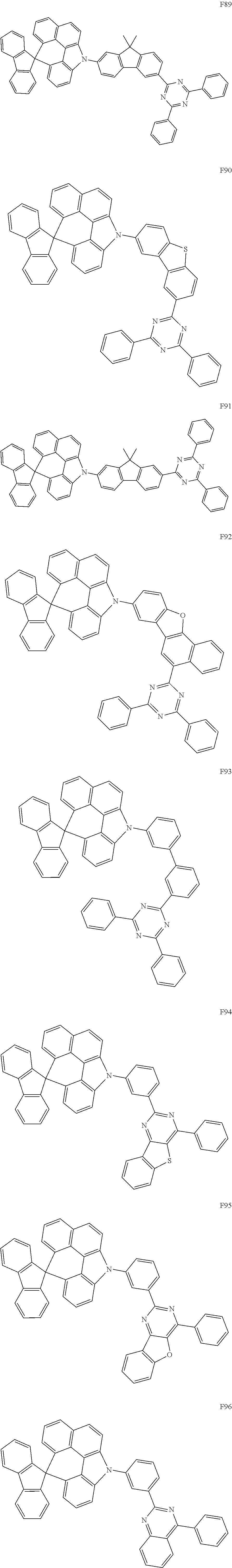

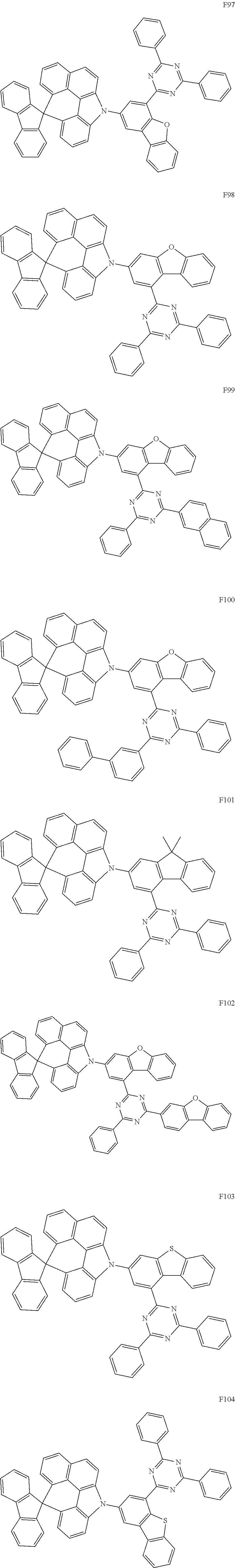

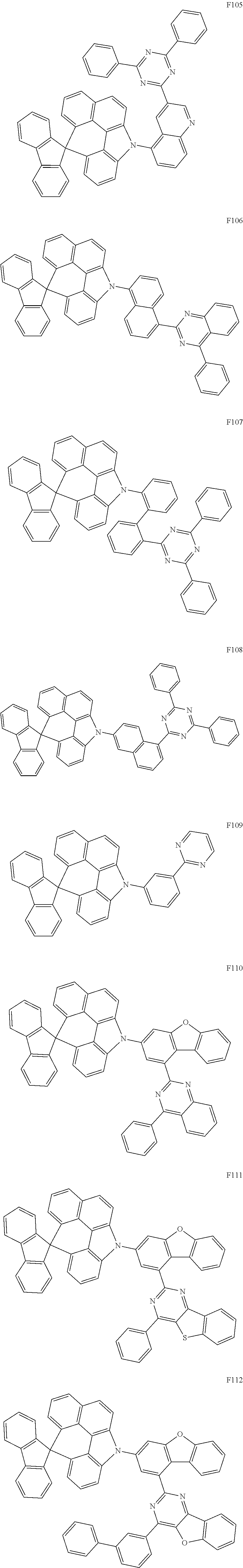

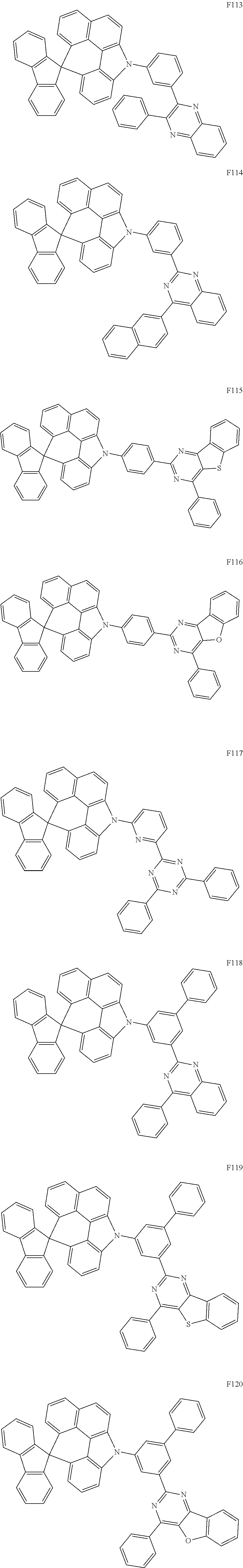

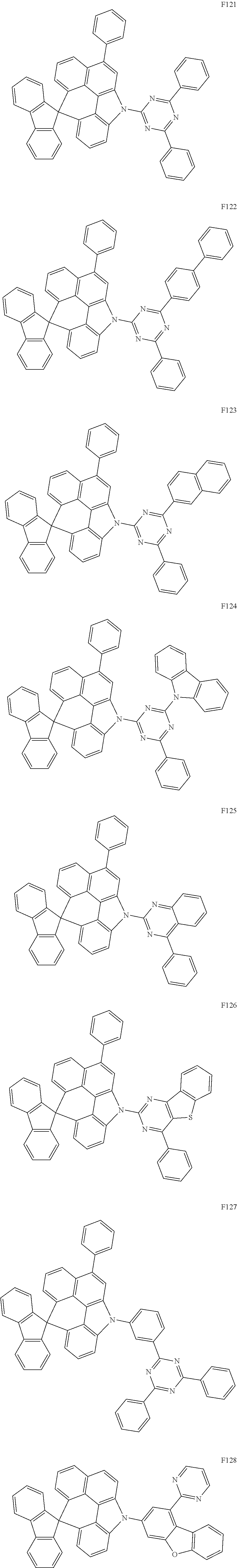

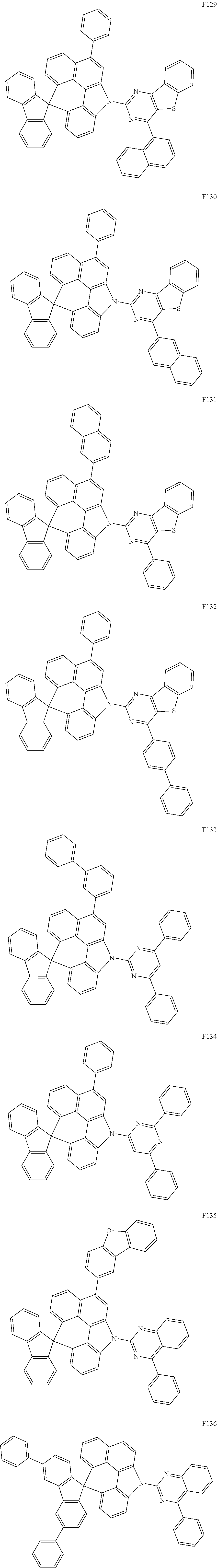

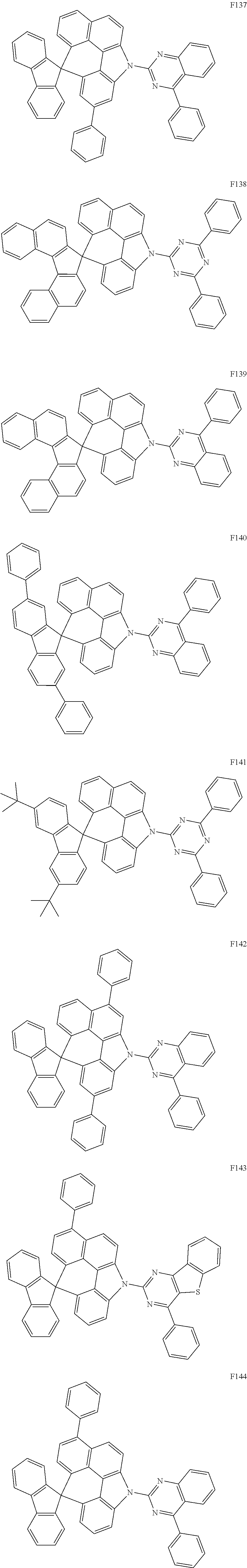

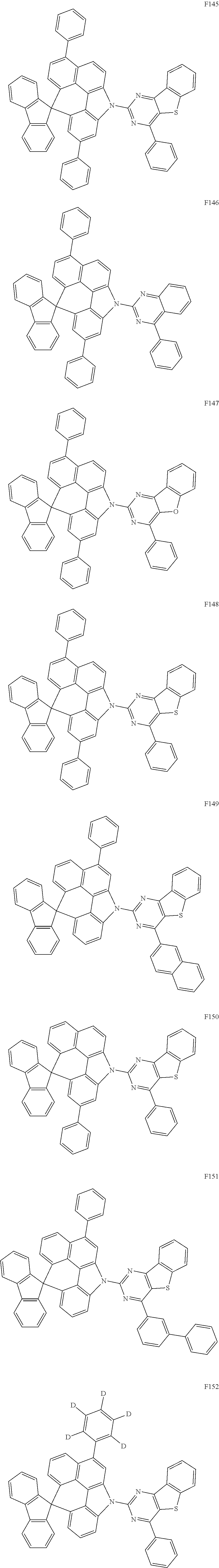

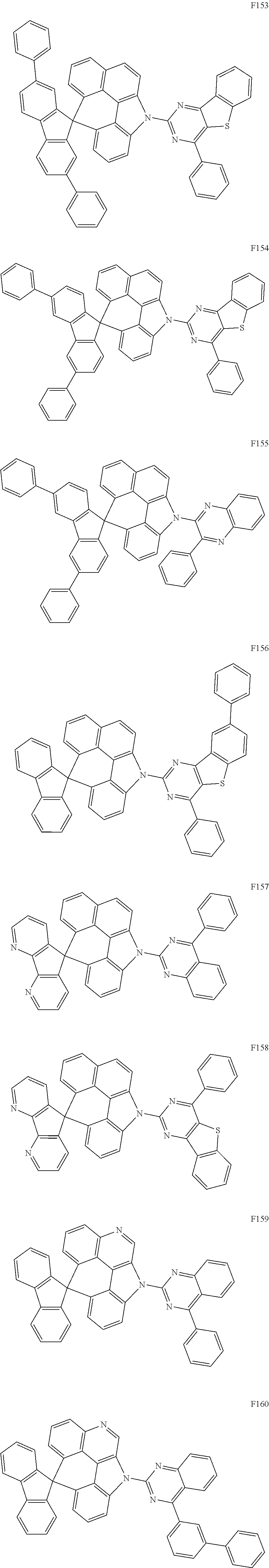

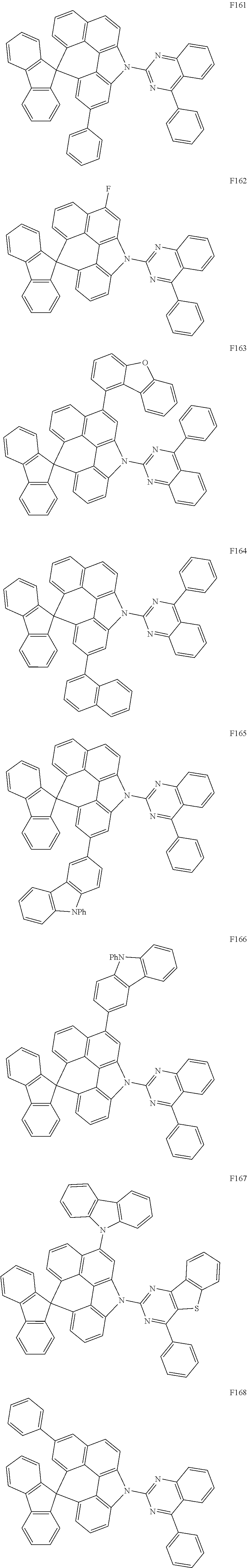

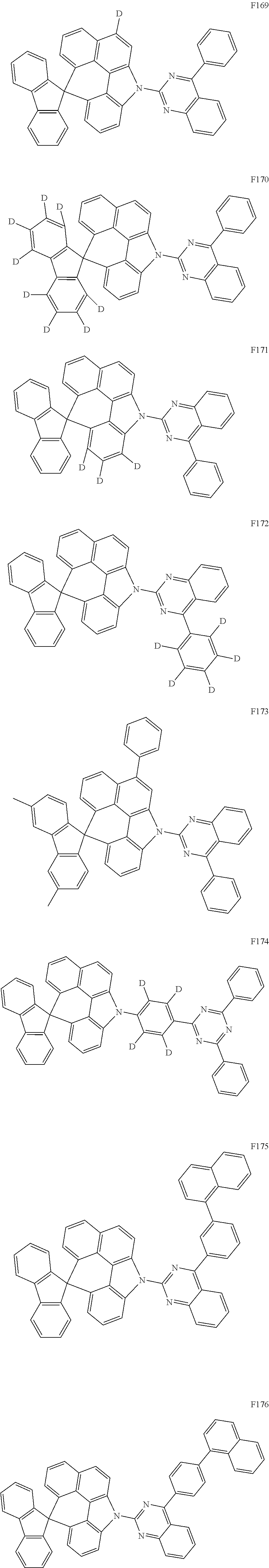

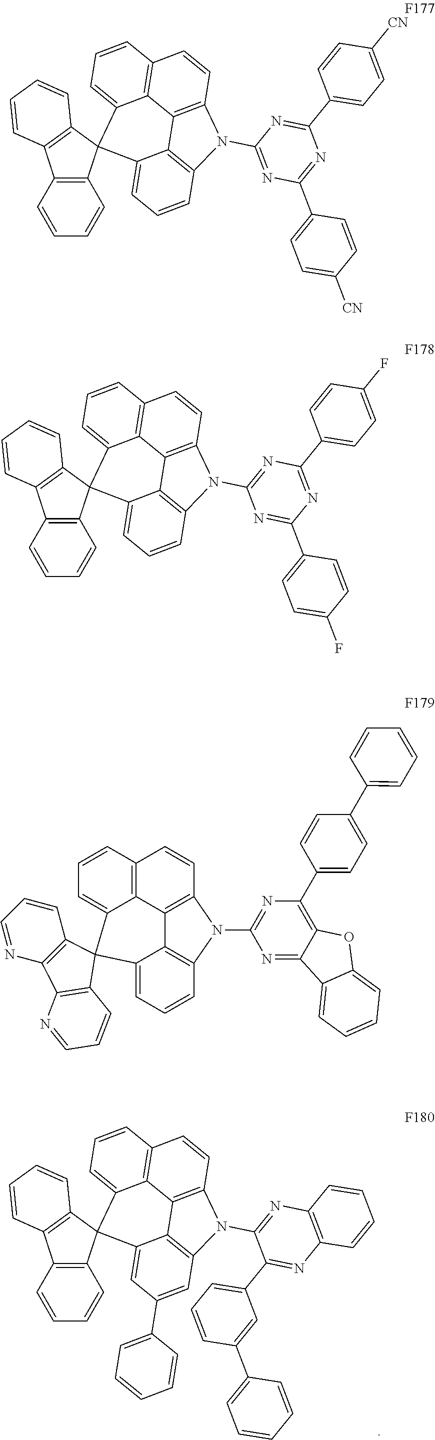 [SMILES: N#Cc1ccc(-c2nc(-c3ccc(C#N)cc3)nc(-n3c4cccc5c4c4c6c(cccc6ccc43)C53c4ccccc4-c4ccccc43)n2)cc1 ; Fc1ccc(-c2nc(-c3ccc(F)cc3)nc(-n3c4cccc5c4c4c6c(cccc6ccc43)C53c4ccccc4-c4ccccc43)n2)cc1 ; c1ccc(-c2ccc(-c3nc(-n4c5cccc6c5c5c7c(cccc7ccc54)C64c5cccnc5-c5ncccc54)nc4c3oc3ccccc34)cc2)cc1 ; c1ccc(-c2cccc(-c3nc4ccccc4nc3-n3c4cc(-c5ccccc5)cc5c4c4c6c(cccc6ccc43)C53c4ccccc4-c4ccccc43)c2)cc1]

In addition, by introducing various substituents into the structure of Formula above, compounds having intrinsic properties of the introduced substituent may be synthesized. For example, by introducing into the core structure a material for a hole injection layer, a material for a hole transport layer, a material for a light-emitting layer, a material for an electron transport layer, and a material for a charge-generating layer used in manufacturing the organic light-emitting device, it is possible to synthesize materials satisfying the conditions required for each organic layer.

In addition, it is possible to finely control the energy band gap by introducing various substituents into the structure of Formula 1, while it is possible to diversify the use of the materials by improving the properties at the interface between organic materials.

In addition, in one embodiment of the present invention, there is provided an organic light-emitting device, comprising a first electrode; a second electrode provided to face the first electrode; and one or more organic layers provided between the first electrode and the second electrode, and wherein one or more of the organic layers comprises the heterocyclic compound represented by Formula 1.

In one embodiment of the present invention, the first electrode may be an anode, and the second electrode may be a cathode.

In another embodiment, the first electrode may be a cathode, and the second electrode may be an anode.

In one embodiment of the present invention, the organic light-emitting device may be a blue organic light-emitting device, and the heterocyclic compound represented by Formula 1 may be used as a material of the blue organic light-emitting device.

In one embodiment of the present invention, the organic light-emitting device may be a green organic light-emitting device, and the heterocyclic compound represented by Formula 1 may be used as a material of the green organic light-emitting device.

In one embodiment of the present invention, the organic light-emitting device may be a red organic light-emitting device, and the heterocyclic compound represented by Formula 1 may be used as a material of the red organic light-emitting device.

In one embodiment of the present invention, the organic light-emitting device may be a blue organic light-emitting device, and the heterocyclic compound represented by Formula 1 may be used as a material for light-emitting layer of the blue organic light-emitting device.

In one embodiment of the present invention, the organic light-emitting device may be a green organic light-emitting device, and the heterocyclic compound represented by Formula 1 may be used as a material for light-emitting layer of the green organic light-emitting device.

In one embodiment of the present invention, the organic light-emitting device may be a red organic light-emitting device, and the heterocyclic compound represented by Formula 1 may be used as a material for light-emitting layer of the red organic light-emitting device.

Specific details of the heterocyclic compound represented by Formula 1 are the same as described above.

The organic light-emitting device of the present invention may be manufactured by conventional methods and materials for manufacturing an organic light-emitting device, except that one or more organic layers are formed using the aforementioned heterocyclic compound.

The heterocyclic compound may be formed into an organic layer by a solution coating method as well as a vacuum deposition method when manufacturing an organic light-emitting device. In this case, the solution coating method refers to, but is not limited to, spin coating, dip coating, inkjet printing, screen printing, spraying, roll coating, and the like.

The organic layer of the organic light-emitting device of the present invention may have a single-layer structure, and may also have a multi-layer structure in which two or more organic layers are stacked. For example, the organic light-emitting device of the present invention may have a structure comprising a hole injection layer, a hole transport layer, a light-emitting layer, an electron transport layer, an electron injection layer, and the like, as an organic layer. However, the structure of the organic light-emitting device is not limited thereto, and may include a smaller number of organic layers.

In addition, one embodiment of the present invention provides a composition for an organic layer of the organic light-emitting device comprising the heterocyclic compound represented by Formula 1.

Specific details of the heterocyclic compound represented by Formula 1 are the same as described above.

The composition for organic layer of the organic light-emitting device may be used when forming an organic material of the organic light-emitting device, and in particular, may be more preferably used when forming a host of the light-emitting layer.

In one embodiment of the present invention, the organic layer includes the heterocyclic compound represented by Formula 1, and may be used together with a phosphorescent dopant.

As the phosphorescent dopant material, those known in the art may be used. For example, phosphorescent dopant materials represented by LL′MX′, LL′L″M, LMX′X″, L₂MX′, and L₃M may be used, but the scope of the present invention is not limited by these examples.
M may be iridium, platinum, osmium, or the like.
L is an anionic bidentate ligand coordinated to M by sp² carbon and a heteroatom, and X may function to trap electrons or holes. Non-limiting examples of L include 2-(1-naphthyl)benzoxazole, (2-phenylbenzoxazole), (2-phenylbenzothiazole), (2-phenylbenzothiazole), (7,8-benzoquinoline), (thiophenylpyrizine), phenylpyridine, benzothiophenylpyrizine, 3-methoxy-2-phenylpyridine, tolylpyridine, and the like. Non-limiting examples of X′ and X″ include acetylacetonate (acac), hexafluoroacetylacetonate, salicylidene, picolinate, 8-hydroxyquinolinate, and the like.

Specific examples of the phosphorescent dopant are shown below, but are not limited to these examples.

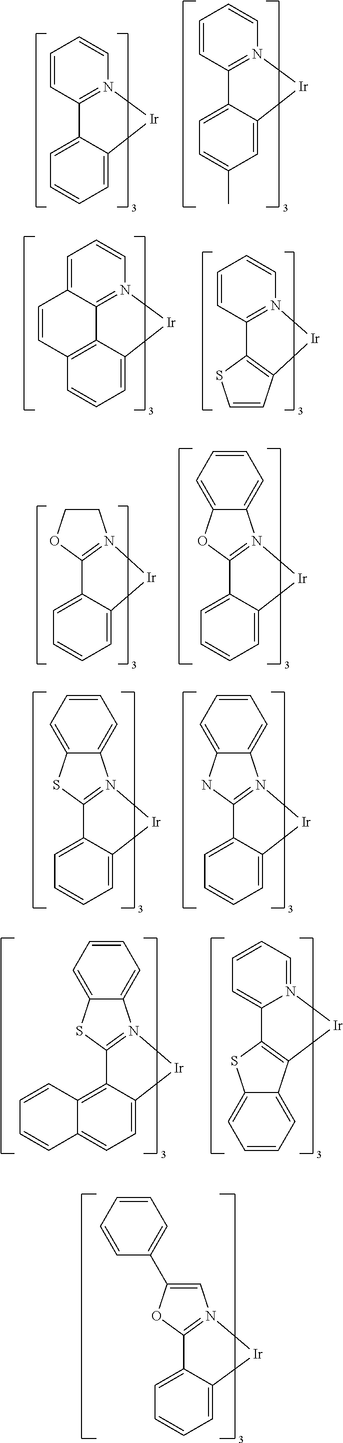

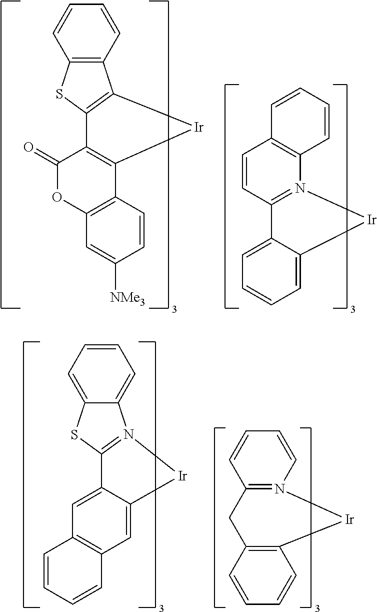

In one embodiment of the present invention, the organic layer includes the heterocyclic compound represented by Formula 1, and may be used together with an iridium-based dopant.

In one embodiment of the present invention, (piq)₂(Ir) (acac), which is a red phosphorescent dopant, may be used as the iridium-based dopant.

In one embodiment of the present invention, the content of the dopant may be 14 to 15%, preferably 3% to 10% based on the entire light-emitting layer.

In the organic light-emitting device according to one embodiment of the present invention, the organic layer may include a light-emitting layer.

In the organic light-emitting device according to another embodiment of the present invention, the organic layer may further include one or two or more layers selected from the group consisting of a light-emitting layer, a hole injection layer, a hole transport layer, an electron injection layer, an electron transport layer, an electron-blocking layer, and a hole-blocking layer.

In the organic light-emitting device according to another embodiment of the present invention, the light-emitting layer may include the heterocyclic compound.

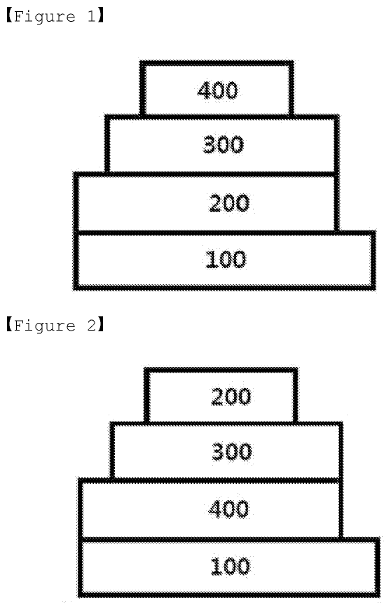

FIGS. 1 to 3 illustrate the stacking order of the electrodes and the organic layers of the organic light-emitting device according to one embodiment of the present invention. However, it is not intended that the scope of the present application be limited by these drawings, and the structure of the organic light-emitting device known in the art may also be applied to the present application.

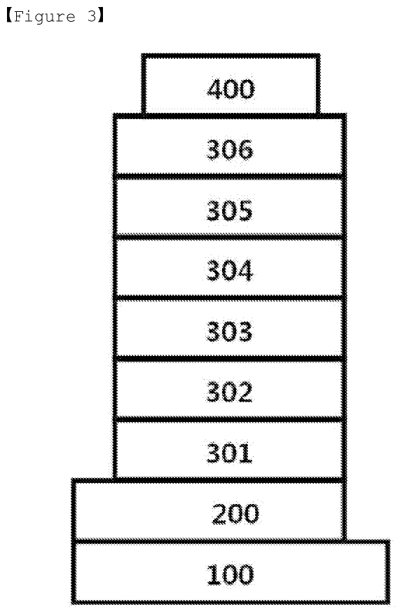

According to FIG. 1, an organic light-emitting device in which an anode 200, an organic layer 300, and a cathode 400 are sequentially stacked on a substrate 100 is shown. However, it is not limited only to such a structure, and an organic light-emitting device in which a cathode, an organic layer, and an anode are sequentially stacked on a substrate may be implemented, as shown in FIG. 2.

FIG. 3 illustrates a case where the organic layer is composed of multiple layers. The organic light-emitting device according to FIG. 3 comprises a hole injection layer 301, a hole transport layer 302, a light-emitting layer 303, a hole-blocking layer 304, an electron transport layer 305, and an electron injection layer 306.

However, the scope of the present application is not limited by the stacked structures as described above, and the remaining layers except for the light-emitting layer may be omitted, if necessary, and other necessary functional layers may be further added.

In one embodiment of the present invention, there is provided a method of manufacturing an organic light-emitting device, comprising the steps of: preparing a substrate; forming a first electrode on the substrate; forming one or more organic layers on the first electrode; and forming a second electrode on the organic layer, and wherein the step of forming the organic layers comprises a step of forming one or more organic layers using the composition for an organic layer according to one embodiment of the present invention.

In one embodiment of the present invention, the step of forming the organic layers may comprise depositing the heterocyclic compound represented by Formula 1 using a thermal vacuum deposition method.

The organic layer including the heterocyclic compound represented by Formula 1 may further comprise another material, if necessary.

The organic layer simultaneously including the heterocyclic compound represented by Formula 1 may further comprise another material, if necessary.

In the organic light-emitting device according to one embodiment of the present invention, the materials other than the heterocyclic compound represented by Formula 1 are exemplified below, but these are for illustrative purposes only and are not intended to limit the scope of the present application, and may be replaced with materials known in the art.

As the anode material, materials having a relatively large work function may be used, and transparent conductive oxides, metals, conductive polymers, or the like may be used. Specific examples of the anode material include, but are not limited to, metals such as vanadium, chromium, copper, zinc, and gold, or alloys thereof; metal oxides such as zinc oxide, indium oxide, indium tin oxide (ITO), and indium zinc oxide (IZO); a combination of metals and oxides such as ZnO: Al or SnO₂: Sb; conductive polymers such as poly(3-methylthiophene), poly[3,4-(ethylene-1,2-dioxy)thiophene] (PEDOT), polypyrrole, and polyaniline; and the like.

As the cathode material, materials having a relatively low work function may be used, and metals, metal oxides, conductive polymers, or the like may be used. Specific examples of the cathode material include, but are not limited to, metals such as magnesium, calcium, sodium, potassium, titanium, indium, yttrium, lithium, gadolinium, aluminum, silver, tin, and lead, or alloys thereof; multilayer-structured materials such as LiF/Al or LiO₂/Al; and the like.

As the hole injection layer material, a known material for the hole injection layer may be used, for example, phthalocyanine compounds such as copper phthalocyanine, and the like, disclosed in U.S. Pat. No. 4,356,429, or starburst-type amine derivatives such as tris(4-carbazolyl-9-ylphenyl)amine (TCTA), 4,4′,4″-tri[phenyl (m-tolyl) amino] triphenylamine (m-MTDATA), 1,3,5-tris[4-(3-methylphenylphenylamino)phenyl]benzene (m-MTDAPB), soluble conductive polymer polyaniline/dodecylbenzenesulfonic acid or poly(3,4-ethylenedioxythiophene)/poly(4-styrenesulfonate), polyaniline/camphor sulfonic acid or polyaniline/poly(4-styrene-sulfonate), or the like, disclosed in Advanced Material, 6, p. 677 (1994) may be used.

As a material for the hole transport layer, a pyrazoline derivative, an arylamine-based derivative, a stilbene derivative, a triphenyldiamine derivative, or the like may be used, and a low-molecular weight or high-molecular weight material may be used.

As a material for the electron transport layer, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone and derivatives thereof, diphenyldicyanoethylene and derivatives thereof, diphenoquinone derivatives, 8-hydroxyquinoline and derivatives thereof, and the like may be used, and high-molecular weight materials as well as low-molecular weight materials may be used.

As a material of the electron injection layer, for example, LiF is typically used in the art, but the present application is not limited thereto.

As a material for the light-emitting layer, a red, green, or blue light-emitting material may be used, and a mixture of two or more light-emitting materials may be used, if necessary. In this case, it is possible to use by depositing two or more light-emitting materials as separate sources, or it is possible to use by pre-mixing and depositing them as a single source. In addition, as a material for the light-emitting layer, a fluorescent material may be used, and a phosphorescent material may also be used. As a material for the light-emitting layer, materials that emit light by combining holes and electrons respectively injected from the anode and the cathode may be used alone, and materials in which the host material and the dopant material together participate in light emission may also be used.

When using by mixing hosts of the material for the light-emitting layer, it is possible to use by mixing hosts of the same type, or it is possible to use by mixing different types of hosts. For example, it is possible to use by selecting any two or more types of n-type host materials or p-type host materials as a host material for the light-emitting layer.

The organic light-emitting device according to one embodiment of the present invention may be a top emission type, a bottom emission type, or a dual emission type depending on the material to be used.

The heterocyclic compound according to one embodiment of the present invention may act on the principle similar to that applied to the organic light-emitting device even in an organic electronic device including an organic solar cell, an organic photoreceptor, an organic transistor, and the like.

Hereinafter, preferred examples will be presented to help the understanding of the present invention, but the following examples are provided not to limit the present invention but to facilitate the understanding of the present invention.

### PREPARATIVE EXAMPLES

### <Preparative Example 1> Preparation of Compounds D1, D6, and

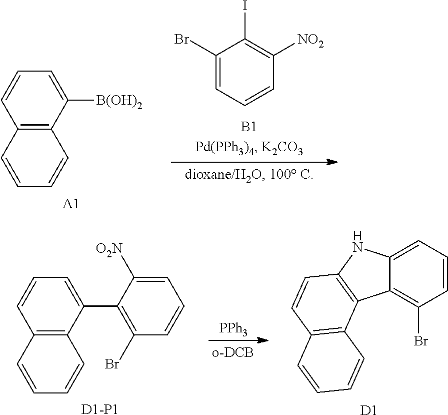

1) Preparation of Compound D1-P1

20 g (116.28 mmol) of Al, 45.7 g (139.53 mmol) of B1, 13.4 g (11.62 mmol) of tetrakis(triphenylphosphine)palladium(0) (Pd(PPh₃)₄), and 32.14 g (232 mmol) of K₂CO₃ were placed in a 1000 mL round-bottom flask, 400 mL of 1,4-dioxane and 80 mL of H₂O were placed therein under a nitrogen atmosphere, and stirred at 100° C. for 12 hours. After completion of the reaction, the reaction temperature was lowered to room temperature and it was washed with water, and then extracted with methylene chloride (MC). The extracted organic solvent was dried over Mg₂SO₄ and then concentrated. Silica-gel column purification and recrystallization were performed to obtain 30.5 g (93.0 mmol, yield 80%) of compound D1-P1 as a white solid compound.

2) Preparation of Compound D1

30.5 g (93.0 mmol) of compound D1-P1 and 60.7 g (232 mmol) of triphenylphosphine (PPh₃) were placed in a 500 mL round-bottom flask, 250 mL of o-dichlorobenzene (o-DCB) was placed therein under a nitrogen atmosphere, and was refluxed for 6 hours. After completion of the reaction, the reaction temperature was lowered to room temperature, it was filtered through a silica-gel filter, DCB was removed with a hexane developing solvent, and then purified by a silica-gel column to obtain 16.2 g (54.9 mmol, yield 59%) of compound D1 as a green solid compound.

The following target compound D was prepared in the same manner as in Preparative Example 1 above, except that intermediates A and B and Compound D-P1 of Table 1 below were used instead of A1, B1, and D1-P1, respectively.

TABLE 1

| Compound | Intermediate | Intermediate | Compound D-P1 | Target Compound D |
|---|---|---|---|---|
| No. | A | B | (Yield) | (Yield) |
| D6 | 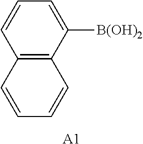 | 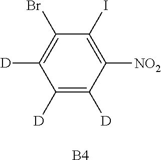 | 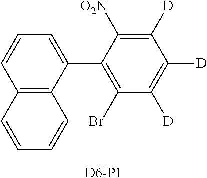 | 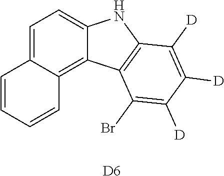 [SMILES: [2H]c1c([2H])c(Br)c2c([nH]c3ccc4ccccc4c32)c1[2H] ; "D6"] |
| | | | (75%) | (62%) |
| D7 | 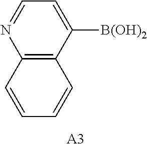 | 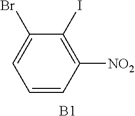 | 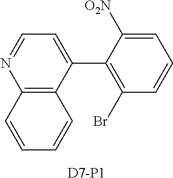 | 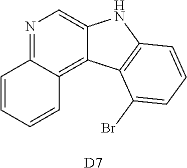 [SMILES: Brc1cccc2[nH]c3cnc4ccccc4c3c12 ; "D7"] |
| | | | (89%) | (38%) |

### <Preparative Example 2> Preparation of Compounds D2, D8, and D9

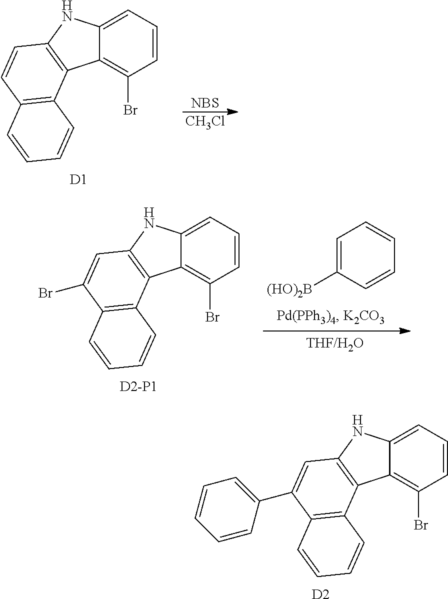

1) Preparation of Compound D2-P1

16.2 g (54.9 mmol) of compound D1 was placed in a 500 ml, round-bottom flask, and 250 mL of MC was placed therein under a nitrogen atmosphere, and then the reaction temperature was lowered to 0° C., and then 11.7 g (65.8 mmol) of N-bromosuccinimide (NBS) solid was added thereto in portions over several times. After stirring at room temperature for 5 hours, the reaction was completed, and then, the solvent was removed under reduced pressure. Thereafter, a hexane solvent was added thereto to precipitate the solid, and then filtered using a paper filter to obtain 16.8 g (45.0 mmol, yield 82%) of compound D2-P1 as a white solid compound.

2) Preparation of Compound D2

16.8 g (45.0 mmol) of compound D2-P1 obtained above, 6.6 g (54 mmol) of phenylboronic acid, 2.6 g (2.25 mmol) of Pd(PPh₃)₄, and 12.4 g (90 mmol) of K₂CO₃ were placed in a 500 mL round-bottom flask, 200 mL of 1,4-dioxane and 50 mL of H₂O were placed therein under a nitrogen atmosphere, and stirred at 120° C. for 12 hours. After completion of the reaction, the reaction temperature was lowered to room temperature, and it was washed with water, and then extracted with MC. The extracted organic solvent was dried over Mg₂SO₄ and then concentrated. Silica gel column purification and recrystallization were performed to obtain 30.5 g (93.0 mmol, yield 65%) of compound D2 as a white solid compound.

The following target compound D was prepared in the same manner as in Preparative Example 2 above, except that the boronic acid of Table 2 below were used instead of the phenylboronic acid.

TABLE 2

| Compound No. | Boronic Acid | Target Compound D | Yield |
|---|---|---|---|
| D8 | 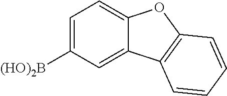 [SMILES: OB(O)c1ccc2oc3ccccc3c2c1] | 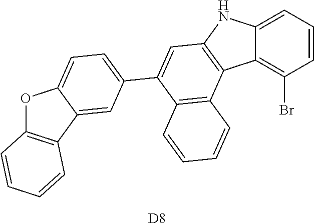 [SMILES: Brc1cccc2[nH]c3cc(-c4ccc5oc6ccccc6c5c4)c4ccccc4c3c12 ; "D8"] | 59% |
| D9 | 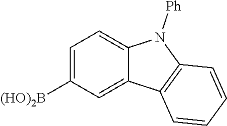 [SMILES: OB(O)c1ccc2c(c1)c1ccccc1n2-c1ccccc1] | 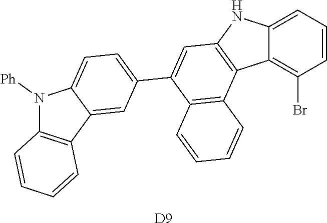 [SMILES: Brc1cccc2[nH]c3cc(-c4ccc5c(c4)c4ccccc4n5-c4ccccc4)c4ccccc4c3c12 ; "D9"] | 65% |

### <Preparative Example 3> Preparation of Compounds D3 and D10

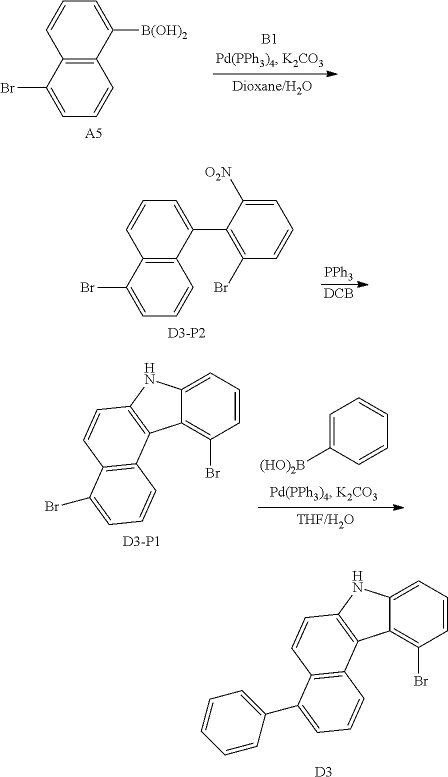

1) Preparation of Compound D3-P2

20 g (1¹6.28 mmol) of compound A5, 45.7 g (139.53 mmol) of B1, 13.4 g (11.62 mmol) of Pd(PPh₃)₄, and 32.14 g (232 mmol) of K₂CO₃ were placed in a 1000 mL round-bottom flask, 400 mL of 1,4-dioxane and 80 mL of H₂O were placed therein under a nitrogen atmosphere, and stirred at 100° C. for 12 hours. After completion of the reaction, the reaction temperature was lowered to room temperature, and it was washed with water, and then extracted with MC. The extracted organic solvent was dried over Mg₂SO₄ and then concentrated. Silica-gel column purification and recrystallization were performed to obtain 31.7 g (77.9 mmol, yield 671) of compound D3-P2 as a white solid compound.

2) Preparation of Compound D3-P1

31.7 g (77.9 mmol) of compound D3-P2 obtained above and 51 g (194.7 mmol) of PPh₃ were placed in a 500 mL round-bottom flask, 200 mL of o-DCB was placed therein under a nitrogen atmosphere, and was refluxed for 12 hours. After completion of the reaction, the reaction temperature was lowered to room temperature, it was filtered through a silica gel filter, DCB was removed with a hexane developing solvent, and then purified by a silica-gel column to obtain 9.6 g (25.7 mmol, yield 33%) of compound D3-P1 as a green solid compound.

3) Preparation of Compound D3

9.6 g (25.7 mmol) of compound D3-P1 obtained above, 3.7 g (30.8 mmol) of phenylboronic acid, 1.5 g (1.28 mmol) of Pd(PPh₃)₄, and 7 g (51 mmol) of K₂CO₃ were placed in a 250 mL round-bottom flask, 80 mL of 1,4-dioxane and 20 mL of H₂O were placed therein under a nitrogen atmosphere, and stirred at 120° C. for 6 hours. After completion of the reaction, the reaction temperature was lowered to room temperature, and it was washed with water, and then extracted with MC. The extracted organic solvent was dried over Mg₂SO₄ and then concentrated. Thereafter, silica-gel column purification and recrystallization were performed to obtain 4 g (10.8 mmol, yield 42%) of compound D3 as a white solid compound.

The following target compound (D) was prepared in the same manner as in Preparative Example 3 above, except that intermediates A and B, Compound D-P2 and Compound D-P1 of Table 3 below were used instead of A5, B1, D3-P2 and D3-P1, respectively.

TABLE 3

| Compound | Intermediate | Intermediate | Compound |
|---|---|---|---|
| No. | A | B | D-P2 (Yield) |
| D10 | 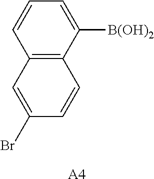 | 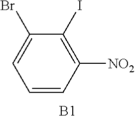 | 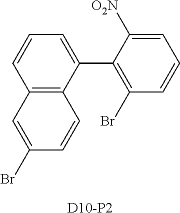 [SMILES: O=[N+]([O-])c1cccc(Br)c1-c1cccc2cc(Br)ccc12 ; "D10-P2"] |
| | | | (74%) |

| Compound | Compound | Target Compound |
|---|---|---|
| No. | D-P1 (Yield) | D (Yield) |
| D10 | 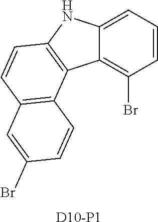 | 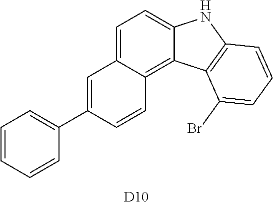 [SMILES: Brc1cccc2[nH]c3ccc4cc(-c5ccccc5)ccc4c3c12 ; "D10"] |
| | (32%) | (39%) |

<Preparative Example 4> Preparation of Compounds D4 and D5

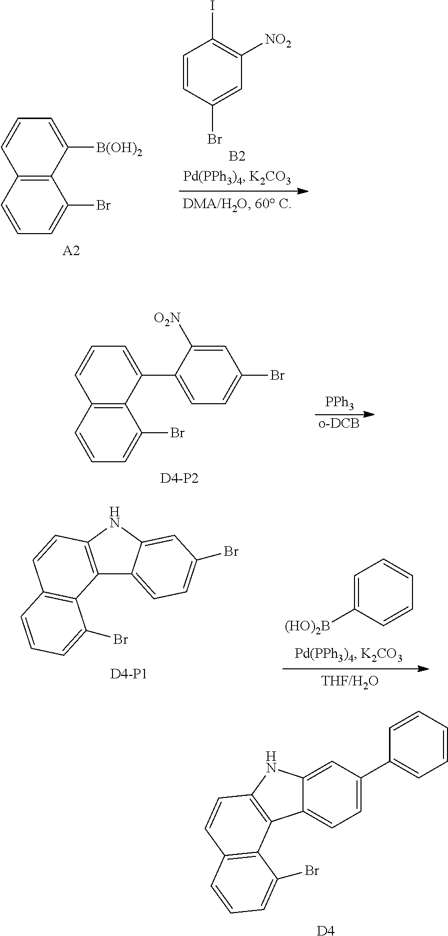

1) Preparation of Compound D4-P2

20 g (116.28 mmol) of compound A5, 45.7 g (139.53 mmol) of B1, 13.4 g (11.62 mmol) of Pd(PPh₃)₄, and 32.14 g (232 mmol) of K₂CO₃ were placed in a 1000 mL round-bottom flask, 400 mL of 1,4-dioxane and 80 mL of H₂O were placed therein under a nitrogen atmosphere, and stirred at 100° C. for 12 hours. After completion of the reaction, the reaction temperature was lowered to room temperature, and it was washed with water, and then extracted with MC. The extracted organic solvent was dried over Mg₂SO₄ and then concentrated. Silica-gel column purification and recrystallization were performed to obtain 29.3 g (72.1 mmol, yield 62%) of compound D3-P2 as a white solid compound.

2) Preparation of Compound D4-P1

29.3 g (72.1 mmol) of compound D3-P2 obtained above and 47 g (180.25 mmol) of PPh₃ were placed in a 500 mL round-bottom flask, 200 mL of o-DCB was placed therein under a nitrogen atmosphere, and was refluxed for 112 hours. After completion of the reaction, the reaction temperature was lowered to room temperature, it was filtered through a silica-gel filter, DCB was removed with a hexane developing solvent, and then purified by a silica-gel column to obtain 11.9 g (31.7 mmol, yield 44%) of compound D4-P1 as a grey solid compound.

3) Preparation of Compound 4

11.9 g (31.7 mmol) of compound D4-P1 obtained above, 4.6 g (38.0 mmol) of phenylboronic acid, 1.8 g (1.58 mmol) of Pd(PPh₃)₄, and 8.7 g (63 mmol) of K₂CO₃ were placed in a 250 mL round-bottom flask, 80 mL of 1,4-dioxane and 20 mL of H₂O were placed therein, and stirred at 120° C. for 6 hours. After completion of the reaction, the reaction temperature was lowered to room temperature, and it was washed with water, and then extracted with MC. The extracted organic solvent was dried over Mg₂SO₄ and then concentrated. Silica-gel column purification and recrystallization were performed to obtain 4 g (10.8 mmol, yield 62%) of compound D4 as a white solid compound.

The following target compound D was prepared in the same manner as in Preparative Example 4 above, except that intermediates A and B, Compound D-P2 and Compound D-P1 of Table 4 below were used instead of A2, B2, D4-P2 and D4-P1, respectively.

TABLE 4

| Compound | Intermediate | Intermediate | Compound |
|---|---|---|---|
| No. | A | B | D-P2 (Yield) |
| D5 | 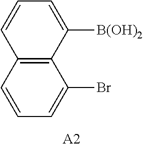 | 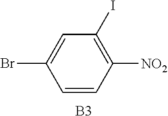 | 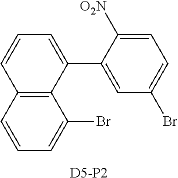 |
| | | | (69%) |
| | Compound | Compound | Target Compound |
| | No. | D-P1 (Yield) | D (Yield) |
| | D5 | 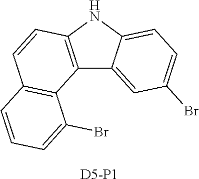 | 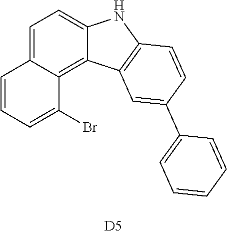 [SMILES: Brc1cccc2ccc3[nH]c4ccc(-c5ccccc5)cc4c3c12 ; "D5"] |
| | | (56%) | (66%) |

<Preparative Example 5> Preparation of Compound F1

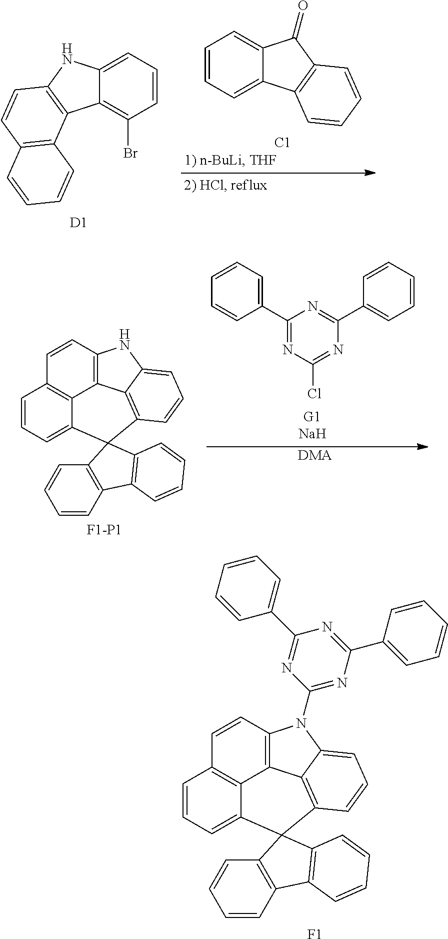

1) Preparation of Compound F1-P1

1.6 g (54 mmol) of compound D1 obtained above was placed in a 250 mL round-bottom flask, and 180 ml of anhydrous tetrahydrofuran (THF, solvent) was added dropwise thereto under a nitrogen atmosphere. 36 mL of 3.0 M n-BuLi/hexane (Hx) was slowly added dropwise while maintaining the temperature of the reaction vessel at −40° C., and then stirred at −40° C. (low temperature) for 4 hours. Thereafter, 14.6 g (81 mmol) of C1 was dissolved in 40 mL of anhydrous THF, and then added dropwise to the reaction; vessel. After stirring at room temperature for 1.0 hours, the completion of the reaction was confirmed by TLC, and then, the solvent was removed under reduced pressure. 37% HCl aqueous solution (40 mL)/acetic acid (40 mL) was added to the reaction vessel, and refluxed for 6 hours, After completion of the reaction, it was washed with water and extracted with MC. The extracted organic solvent was dried over Mg₂SO₄ and then concentrated. Silica-gel column purification and recrystallization were performed to obtain 8.4 g (22.14 mmol, yield 41%) of compound F1-P1 as a white solid

2) Preparation of Compound F1

4.2 g (114 mmol) of compound F1-P1 obtained above and 4.4 g (16.5 mmol) of G1 were placed in a 250 mL round-bottom flask, 50 mL of dimethylacetamide (DMA) was placed therein, and 0.79 g (33 mmol) of NaH was added thereto in portions over several times. Thereafter, the reaction mixture was stirred at 140° C. for 6 hours. After completion of the reaction, the reaction temperature was lowered to room temperature, and it was washed with water, and then extracted with MC. The extracted organic solvent was dried over Mg₂O₄ and then concentrated. Silica-gel column purification and recrystallization were performed to obtain 10.1 g (10.1 mmol, yield 92%) of compound F1 as a white solid compound.

The following target compound k was prepared in the same manner as in Preparative Example 5 above, except that intermediates D, C, and G of Table 5 below were used instead of D1, C1, and G1, respectively.

TABLE 5

| Com- | | | | |
|---|---|---|---|---|
| pound | Intermediate | Intermediate | | |
| No. | D | C | Intermediate G | Target Compound F |
| F1 | 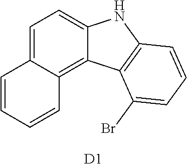 | 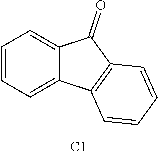 | 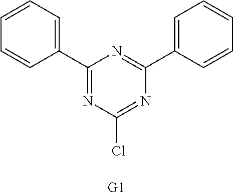 [SMILES: Clc1nc(-c2ccccc2)nc(-c2ccccc2)n1] | 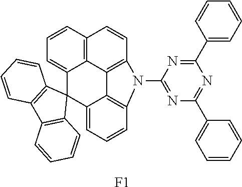 [SMILES: c1ccc(-c2nc(-c3ccccc3)nc(-n3c4cccc5c4c4c6c(cccc6ccc43)C53c4ccccc4-c4ccccc43)n2)cc1 ; "F1"] |
| F2 | 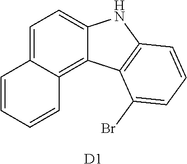 | 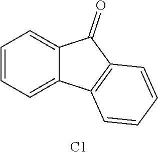 | 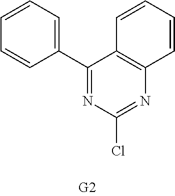 [SMILES: Clc1nc(-c2ccccc2)c2ccccc2n1] | 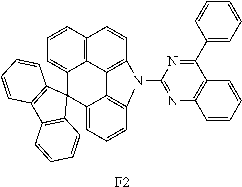 [SMILES: c1ccc(-c2nc(-n3c4cccc5c4c4c6c(cccc6ccc43)C53c4ccccc4-c4ccccc43)nc3ccccc23)cc1 ; "F2"] |
| F3 | 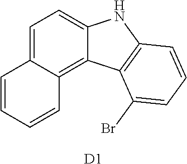 | 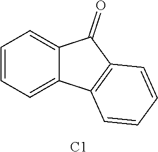 | 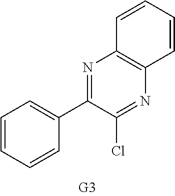 [SMILES: Clc1nc2ccccc2nc1-c1ccccc1] | 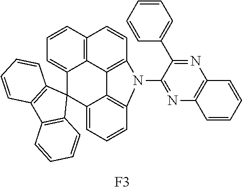 [SMILES: c1ccc(-c2nc3ccccc3nc2-n2c3cccc4c3c3c5c(cccc5ccc32)C42c3ccccc3-c3ccccc32)cc1 ; "F3"] |
| F4 | 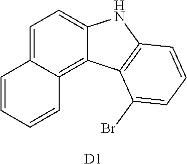 | 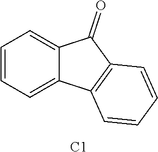 | 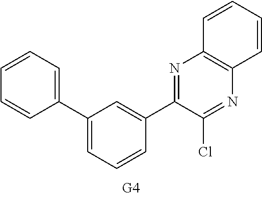 [SMILES: Clc1nc2ccccc2nc1-c1cccc(-c2ccccc2)c1] | 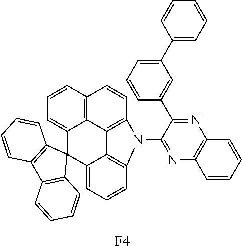 [SMILES: c1ccc(-c2cccc(-c3nc4ccccc4nc3-n3c4cccc5c4c4c6c(cccc6ccc43)C53c4ccccc4-c4ccccc43)c2)cc1 ; "F4"] |
| F5 | 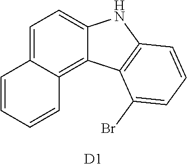 | 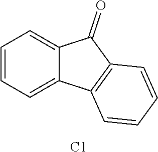 | 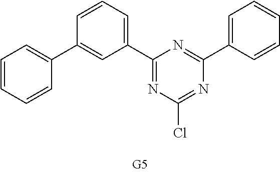 [SMILES: Clc1nc(-c2ccccc2)nc(-c2cccc(-c3ccccc3)c2)n1] | 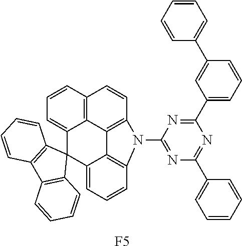 [SMILES: c1ccc(-c2cccc(-c3nc(-c4ccccc4)nc(-n4c5cccc6c5c5c7c(cccc7ccc54)C64c5ccccc5-c5ccccc54)n3)c2)cc1 ; "F5"] |
| F8 | 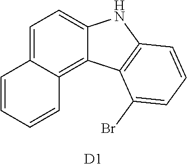 | 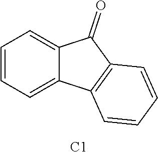 | 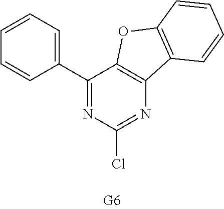 [SMILES: Clc1nc(-c2ccccc2)c2oc3ccccc3c2n1] | 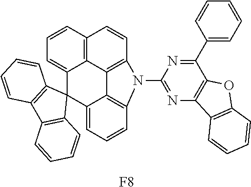 [SMILES: c1ccc(-c2nc(-n3c4cccc5c4c4c6c(cccc6ccc43)C53c4ccccc4-c4ccccc43)nc3c2oc2ccccc23)cc1 ; "F8"] |
| F9 | 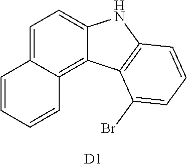 | 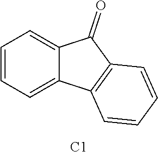 | 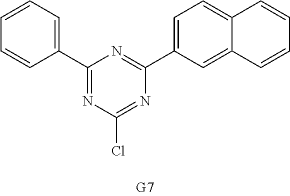 [SMILES: Clc1nc(-c2ccccc2)nc(-c2ccc3ccccc3c2)n1] | 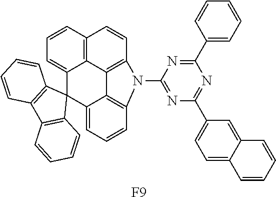 [SMILES: c1ccc(-c2nc(-c3ccc4ccccc4c3)nc(-n3c4cccc5c4c4c6c(cccc6ccc43)C53c4ccccc4-c4ccccc43)n2)cc1 ; "F9"] |
| F12 | 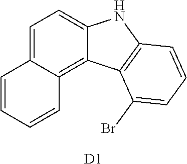 | 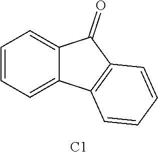 | 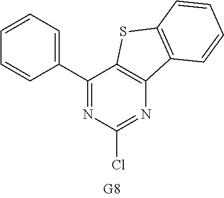 [SMILES: Clc1nc(-c2ccccc2)c2sc3ccccc3c2n1] | 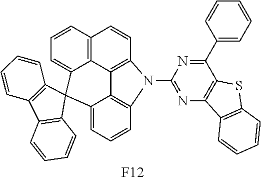 |
| F20 | 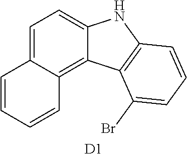 | 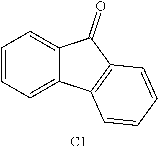 | 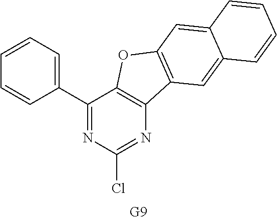 [SMILES: Clc1nc(-c2ccccc2)c2oc3cc4ccccc4cc3c2n1] | 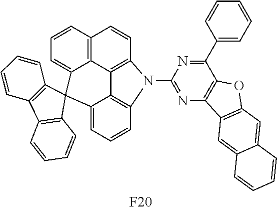 [SMILES: c1ccc(-c2nc(-n3c4cccc5c4c4c6c(cccc6ccc43)C53c4ccccc4-c4ccccc43)nc3c2oc2cc4ccccc4cc23)cc1 ; "F20"] |
| F23 | 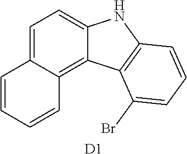 | 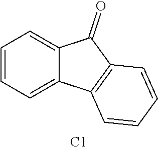 | 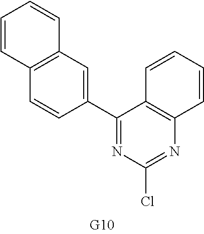 [SMILES: Clc1nc(-c2ccc3ccccc3c2)c2ccccc2n1] | 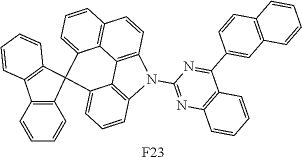 [SMILES: c1ccc2c(c1)-c1ccccc1C21c2cccc3ccc4c(c23)c2c1cccc2n4-c1nc(-c2ccc3ccccc3c2)c2ccccc2n1 ; "F23"] |
| F24 | 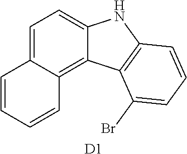 | 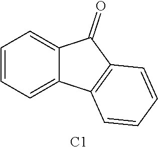 | 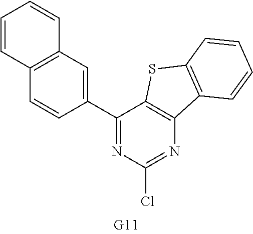 [SMILES: Clc1nc(-c2ccc3ccccc3c2)c2sc3ccccc3c2n1] | 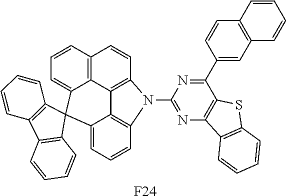 [SMILES: c1ccc2c(c1)-c1ccccc1C21c2cccc3ccc4c(c23)c2c1cccc2n4-c1nc(-c2ccc3ccccc3c2)c2sc3ccccc3c2n1 ; "F24"] |
| F25 | 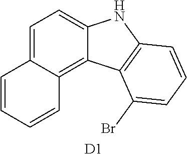 | 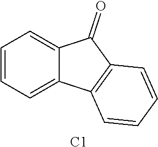 | 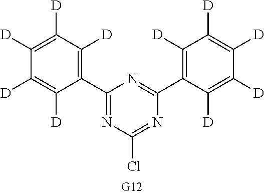 [SMILES: [2H]c1c([2H])c([2H])c(-c2nc(Cl)nc(-c3c([2H])c([2H])c([2H])c([2H])c3[2H])n2)c([2H])c1[2H]] | 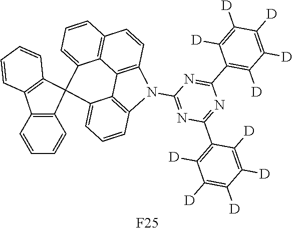 [SMILES: [2H]c1c([2H])c([2H])c(-c2nc(-c3c([2H])c([2H])c([2H])c([2H])c3[2H])nc(-n3c4cccc5c4c4c6c(cccc6ccc43)C53c4ccccc4-c4ccccc43)n2)c([2H])c1[2H] ; "F25"] |
| F38 | 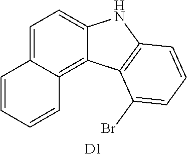 | 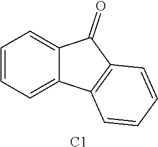 | 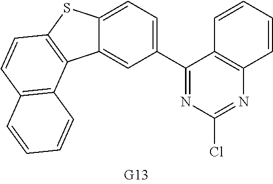 [SMILES: Clc1nc(-c2ccc3sc4ccc5ccccc5c4c3c2)c2ccccc2n1] | 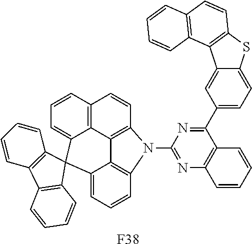 |
| F46 | 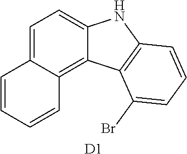 | 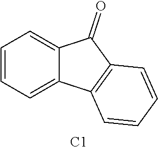 | 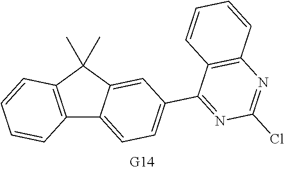 [SMILES: CC1(C)c2ccccc2-c2ccc(-c3nc(Cl)nc4ccccc34)cc21] | 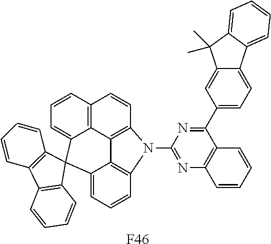 [SMILES: CC1(C)c2ccccc2-c2ccc(-c3nc(-n4c5cccc6c5c5c7c(cccc7ccc54)C64c5ccccc5-c5ccccc54)nc4ccccc34)cc21 ; "F46"] |
| F48 | 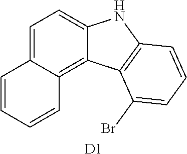 | 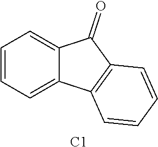 | 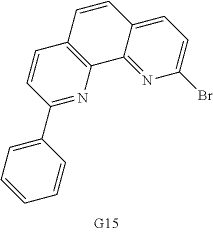 [SMILES: Brc1ccc2ccc3ccc(-c4ccccc4)nc3c2n1] | 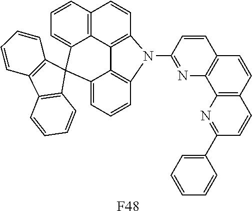 [SMILES: c1ccc(-c2ccc3ccc4ccc(-n5c6cccc7c6c6c8c(cccc8ccc65)C75c6ccccc6-c6ccccc65)nc4c3n2)cc1 ; "F48"] |
| F50 | 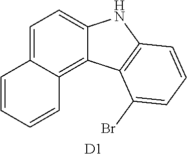 | 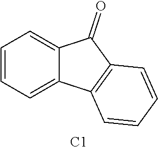 | 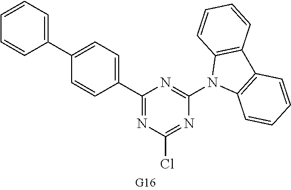 [SMILES: Clc1nc(-c2ccc(-c3ccccc3)cc2)nc(-n2c3ccccc3c3ccccc32)n1] | 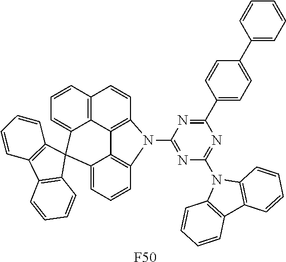 [SMILES: c1ccc(-c2ccc(-c3nc(-n4c5ccccc5c5ccccc54)nc(-n4c5cccc6c5c5c7c(cccc7ccc54)C64c5ccccc5-c5ccccc54)n3)cc2)cc1 ; "F50"] |
| F56 | 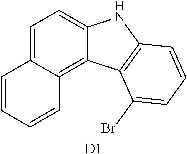 | 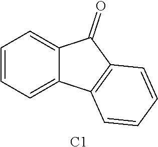 | 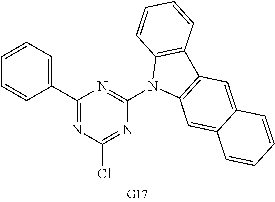 [SMILES: Clc1nc(-c2ccccc2)nc(-n2c3ccccc3c3cc4ccccc4cc32)n1] | 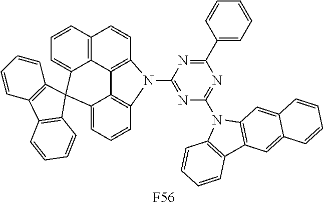 [SMILES: c1ccc(-c2nc(-n3c4ccccc4c4cc5ccccc5cc43)nc(-n3c4cccc5c4c4c6c(cccc6ccc43)C53c4ccccc4-c4ccccc43)n2)cc1 ; "F56"] |
| F62 | 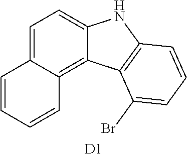 | 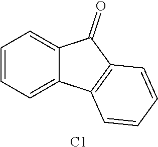 | 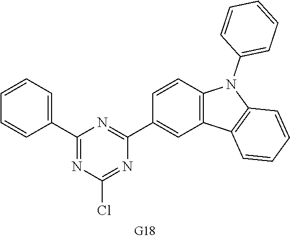 [SMILES: Clc1nc(-c2ccccc2)nc(-c2ccc3c(c2)c2ccccc2n3-c2ccccc2)n1] | 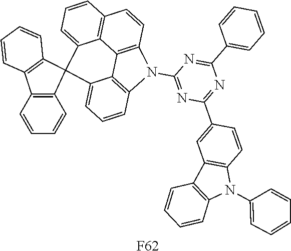 [SMILES: c1ccc(-c2nc(-c3ccc4c(c3)c3ccccc3n4-c3ccccc3)nc(-n3c4cccc5c4c4c6c(cccc6ccc43)C53c4ccccc4-c4ccccc43)n2)cc1 ; "F62"] |
| F63 | 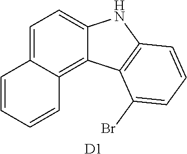 | 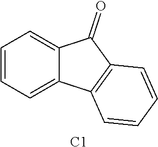 | 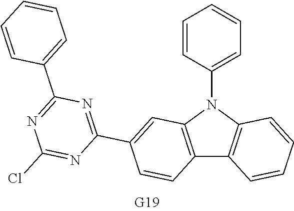 [SMILES: Clc1nc(-c2ccccc2)nc(-c2ccc3c4ccccc4n(-c4ccccc4)c3c2)n1] | 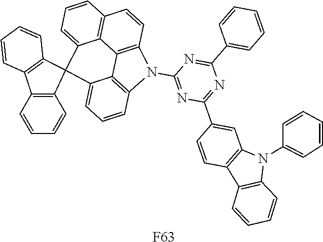 [SMILES: c1ccc(-c2nc(-c3ccc4c5ccccc5n(-c5ccccc5)c4c3)nc(-n3c4cccc5c4c4c6c(cccc6ccc43)C53c4ccccc4-c4ccccc43)n2)cc1 ; "F63"] |
| F121 | 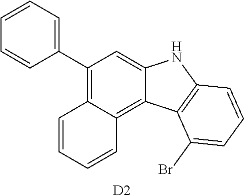 | 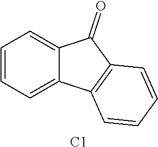 | 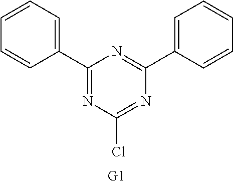 [SMILES: Clc1nc(-c2ccccc2)nc(-c2ccccc2)n1] | 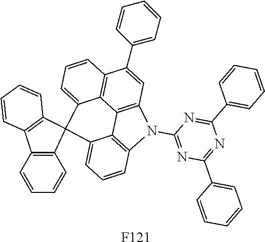 [SMILES: c1ccc(-c2nc(-c3ccccc3)nc(-n3c4cccc5c4c4c6c(cccc6c(-c6ccccc6)cc43)C53c4ccccc4-c4ccccc43)n2)cc1 ; "F121"] |
| F125 | 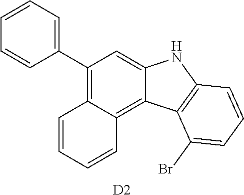 | 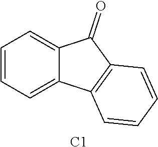 | 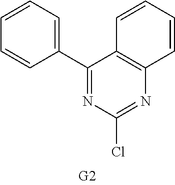 [SMILES: Clc1nc(-c2ccccc2)c2ccccc2n1] | 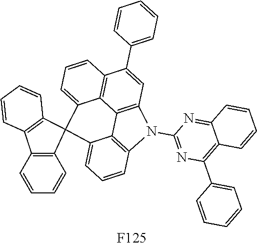 [SMILES: c1ccc(-c2nc(-n3c4cccc5c4c4c6c(cccc6c(-c6ccccc6)cc43)C53c4ccccc4-c4ccccc43)nc3ccccc23)cc1 ; "F125"] |
| F132 | 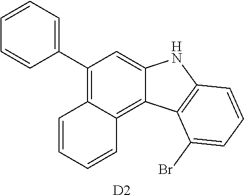 | 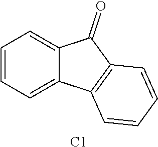 | 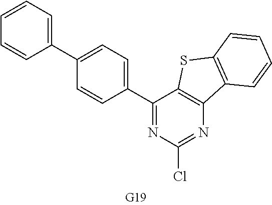 [SMILES: Clc1nc(-c2ccc(-c3ccccc3)cc2)c2sc3ccccc3c2n1] | 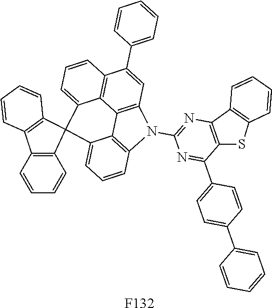 [SMILES: c1ccc(-c2ccc(-c3nc(-n4c5cccc6c5c5c7c(cccc7c(-c7ccccc7)cc54)C64c5ccccc5-c5ccccc54)nc4c3sc3ccccc34)cc2)cc1 ; "F132"] |
| F135 | 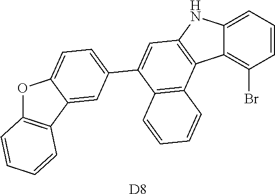 [SMILES: Brc1cccc2[nH]c3cc(-c4ccc5oc6ccccc6c5c4)c4ccccc4c3c12 ; "D8"] | 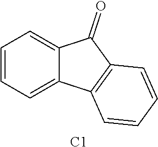 | 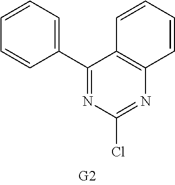 [SMILES: Clc1nc(-c2ccccc2)c2ccccc2n1] | 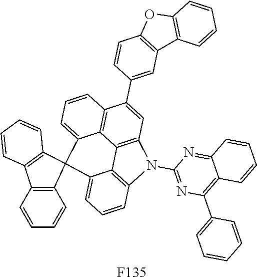 [SMILES: c1ccc(-c2nc(-n3c4cccc5c4c4c6c(cccc6c(-c6ccc7oc8ccccc8c7c6)cc43)C53c4ccccc4-c4ccccc43)nc3ccccc23)cc1 ; "F135"] |
| F136 | 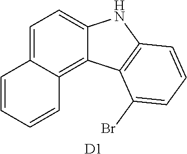 | 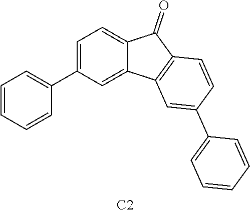 [SMILES: O=C1c2ccc(-c3ccccc3)cc2-c2cc(-c3ccccc3)ccc21 ; "C2"] | 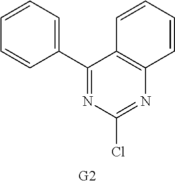 [SMILES: Clc1nc(-c2ccccc2)c2ccccc2n1] | 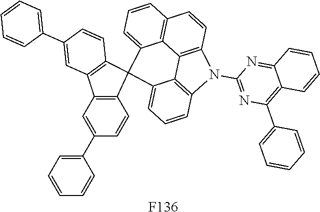 [SMILES: c1ccc(-c2ccc3c(c2)-c2cc(-c4ccccc4)ccc2C32c3cccc4ccc5c(c34)c3c2cccc3n5-c2nc(-c3ccccc3)c3ccccc3n2)cc1 ; "F136"] |
| F139 | 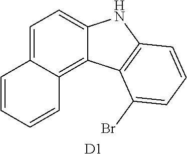 | 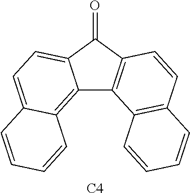 | 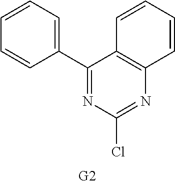 [SMILES: Clc1nc(-c2ccccc2)c2ccccc2n1] | 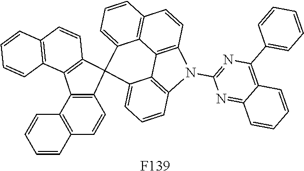 [SMILES: c1ccc(-c2nc(-n3c4cccc5c4c4c6c(cccc6ccc43)C53c4ccc5ccccc5c4-c4c3ccc3ccccc43)nc3ccccc23)cc1 ; "F139"] |
| F140 | 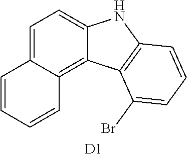 | 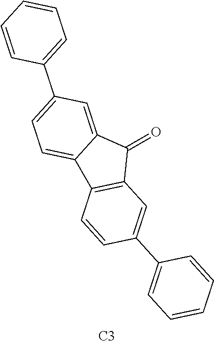 [SMILES: O=C1c2cc(-c3ccccc3)ccc2-c2ccc(-c3ccccc3)cc21 ; "C3"] | 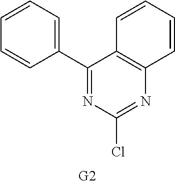 [SMILES: Clc1nc(-c2ccccc2)c2ccccc2n1] | 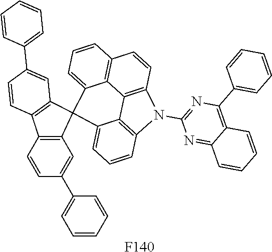 |
| F141 | 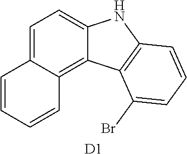 | 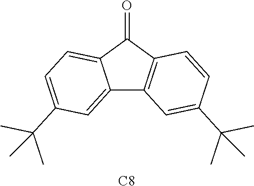 | 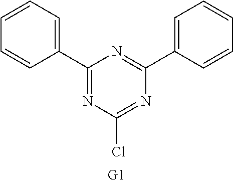 [SMILES: Clc1nc(-c2ccccc2)nc(-c2ccccc2)n1] | 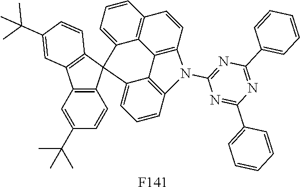 [SMILES: CC(C)(C)c1ccc2c(c1)-c1cc(C(C)(C)C)ccc1C21c2cccc3ccc4c(c23)c2c1cccc2n4-c1nc(-c2ccccc2)nc(-c2ccccc2)n1 ; "F141"] |
| F143 | 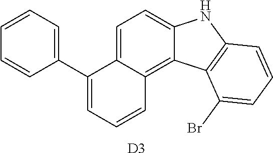 | 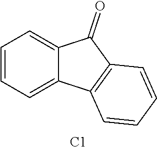 | 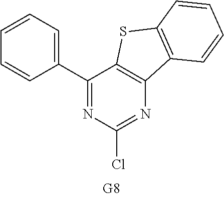 [SMILES: Clc1nc(-c2ccccc2)c2sc3ccccc3c2n1] | 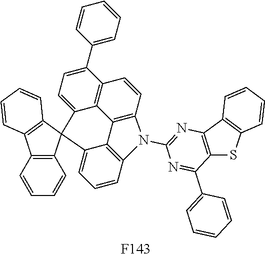 [SMILES: c1ccc(-c2ccc3c4c2ccc2c4c4c(cccc4n2-c2nc(-c4ccccc4)c4sc5ccccc5c4n2)C32c3ccccc3-c3ccccc32)cc1 ; "F143"] |
| F152 | 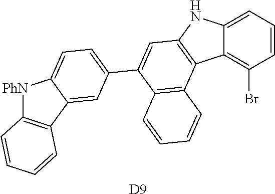 | 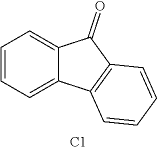 | 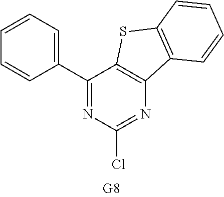 [SMILES: Clc1nc(-c2ccccc2)c2sc3ccccc3c2n1] | 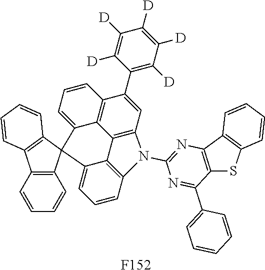 [SMILES: [2H]c1c([2H])c([2H])c(-c2cc3c4c5c(cccc25)C2(c5ccccc5-c5ccccc52)c2cccc(c24)n3-c2nc(-c3ccccc3)c3sc4ccccc4c3n2)c([2H])c1[2H] ; "F152"] |
| F157 | 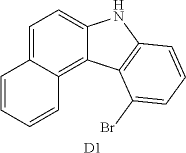 | 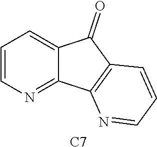 [SMILES: O=C1c2cccnc2-c2ncccc21 ; "C7"] | 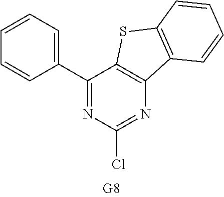 [SMILES: Clc1nc(-c2ccccc2)c2sc3ccccc3c2n1] | 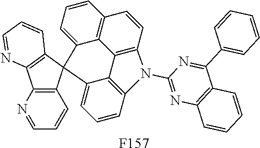 |
| F158 | 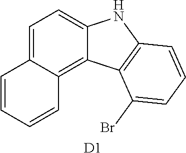 | 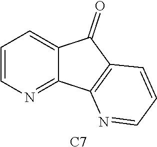 [SMILES: O=C1c2cccnc2-c2ncccc21 ; "C7"] | 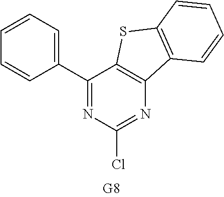 [SMILES: Clc1nc(-c2ccccc2)c2sc3ccccc3c2n1] | 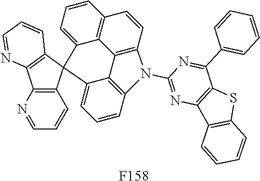 [SMILES: c1ccc(-c2nc(-n3c4cccc5c4c4c6c(cccc6ccc43)C53c4cccnc4-c4ncccc43)nc3c2sc2ccccc23)cc1 ; "F158"] |
| F159 | 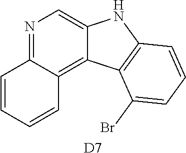 | 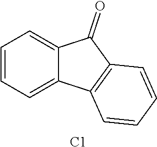 | 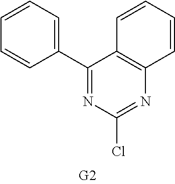 [SMILES: Clc1nc(-c2ccccc2)c2ccccc2n1] | 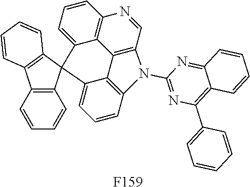 [SMILES: c1ccc(-c2nc(-n3c4cccc5c4c4c6c(cccc6ncc43)C53c4ccccc4-c4ccccc43)nc3ccccc23)cc1 ; "F159"] |
| F166 | 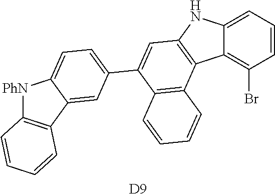 | 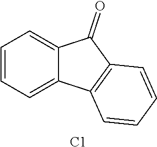 | 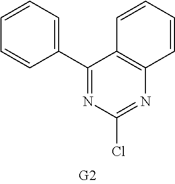 [SMILES: Clc1nc(-c2ccccc2)c2ccccc2n1] | 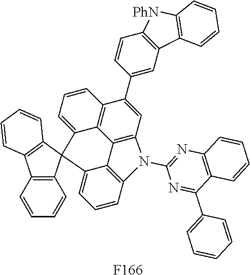 |
| F168 | 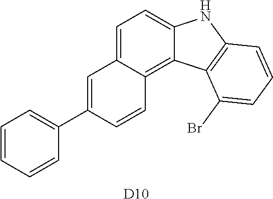 [SMILES: Brc1cccc2[nH]c3ccc4cc(-c5ccccc5)ccc4c3c12 ; "D10"] | 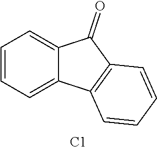 | 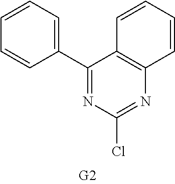 [SMILES: Clc1nc(-c2ccccc2)c2ccccc2n1] | 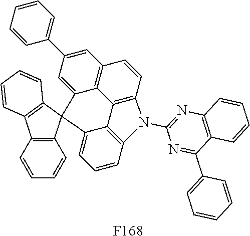 [SMILES: c1ccc(-c2cc3c4c(ccc5c4c4c(cccc4n5-c4nc(-c5ccccc5)c5ccccc5n4)C34c3ccccc3-c3ccccc34)c2)cc1 ; "F168"] |
| F170 | 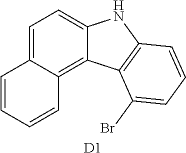 | 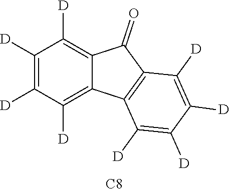 [SMILES: [2H]c1c([2H])c([2H])c2c(c1[2H])C(=O)c1c([2H])c([2H])c([2H])c([2H])c1-2 ; "C8"] | 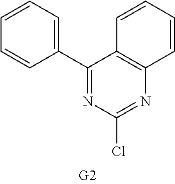 [SMILES: Clc1nc(-c2ccccc2)c2ccccc2n1] | 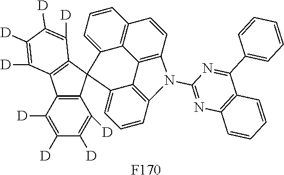 [SMILES: [2H]c1c([2H])c([2H])c2c(c1[2H])-c1c([2H])c([2H])c([2H])c([2H])c1C21c2cccc3ccc4c(c23)c2c1cccc2n4-c1nc(-c2ccccc2)c2ccccc2n1 ; "F170"] |
| F171 | 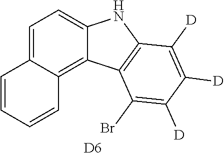 | 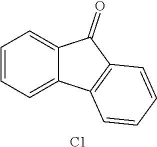 | 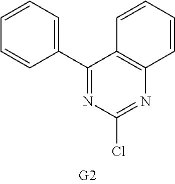 [SMILES: Clc1nc(-c2ccccc2)c2ccccc2n1] | 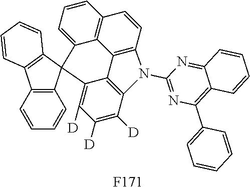 [SMILES: [2H]c1c([2H])c([2H])c2c3c1C1(c4ccccc4-c4ccccc41)c1cccc4ccc(c3c14)n2-c1nc(-c2ccccc2)c2ccccc2n1 ; "F171"] |
| F177 | 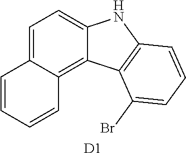 | 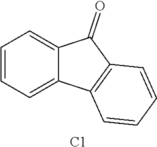 | 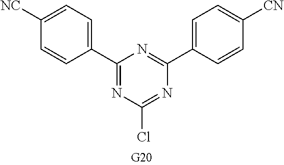 [SMILES: N#Cc1ccc(-c2nc(Cl)nc(-c3ccc(C#N)cc3)n2)cc1] | 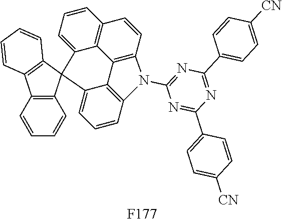 [SMILES: N#Cc1ccc(-c2nc(-c3ccc(C#N)cc3)nc(-n3c4cccc5c4c4c6c(cccc6ccc43)C53c4ccccc4-c4ccccc43)n2)cc1 ; "F177"] |
| F178 | 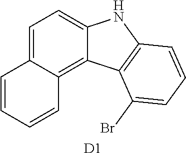 | 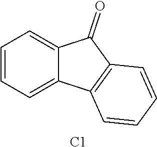 | 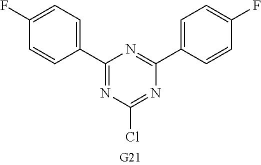 [SMILES: Fc1ccc(-c2nc(Cl)nc(-c3ccc(F)cc3)n2)cc1] | 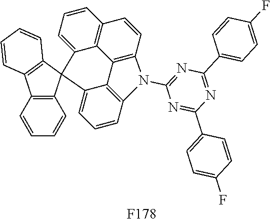 [SMILES: Fc1ccc(-c2nc(-c3ccc(F)cc3)nc(-n3c4cccc5c4c4c6c(cccc6ccc43)C53c4ccccc4-c4ccccc43)n2)cc1 ; "F178"] |

### <Preparative Example 6> Preparation of Compound 773

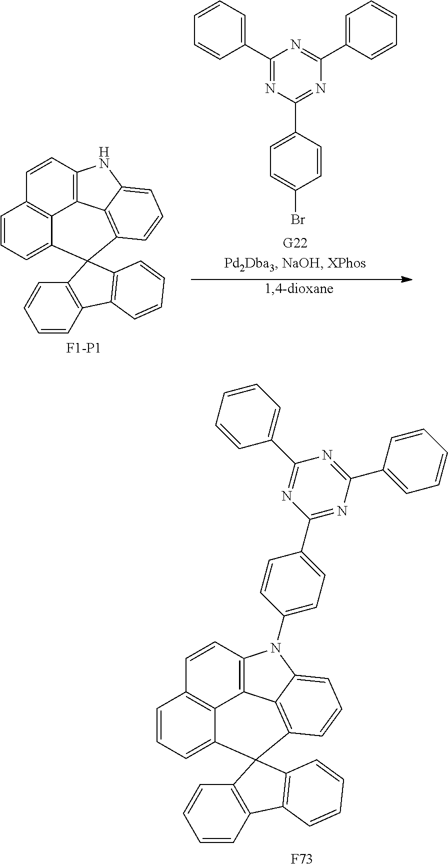

1) Preparation of Compound F1-P1

16 g (54 mmol) of compound DA obtained above was placed in a 250 mL round-bottom flask, and 180 mL of anhydrous tetrahydrofuran (THF, solvent) was added dropwise thereto under a nitrogen atmosphere. 36 mL of 3.0 M n-BuLi/hexane (Ox) was slowly added dropwise while maintaining the temperature of the reaction vessel at −40° C., and then stirred at −40° C. (low temperature, for 4 hours. Thereafter, 14.6 g (81 mmol) of C1 was dissolved in 40 mL of anhydrous THF, and then added dropwise to the reaction vessel. After stirring at room temperature for 10 hours, the completion of the reaction was confirmed by TLC, and then, the solvent was removed under reduced pressure. 37% HCl aqueous solution (40 mL)/acetic acid (40 mL) was added to the reaction vessel, and refluxed for 6 hours. After completion of the reaction, it was washed with water and extracted with MC. The extracted organic solvent was dried over Mg₂SO₄ and then concentrated. Silica-gel column purification and recrystallization were performed to obtain 8.4 g (22.14 mmol, yield 41%) of compound F1-P1 as a white solid compound.

2) Preparation of Compound F73

4.2 g (11 mmol) of compound F1-P1 obtained above, 1 g (1.1 mmol) of tris(dibenzylideneacetone)dipalladium(0) (Pd dbas), 1.3 g (33 mmol) of NaOH, 2.0 g (4.4 mmol) of dicyclohexyl(2′,4′,6′-triisopropyl-[1,1′-biphenyl]-2-yl)phosphine (Xphos), and 6.4 g (16.5 mmol) of G22 were placed in a 250 mL round-bottom flask, and 50 mL of 1,4-dioxane was placed therein. The reaction mixture was stirred at 120° C. for 2 hours. After completion of the reaction, the reaction temperature was lowered to room temperature, and it was washed with water, and then extracted with MC. The extracted organic solvent was dried over Mg₂SO₄ and then concentrated. Silica-gel column purification and recrystallization were performed to obtain 4 g (10.8 mmol, yield 621) of compound F73 as a yellow solid compound.

The following target compound F was prepared in the same manner as in Preparative Example 6 above, except that intermediate G of Table 6 below was used instead of G22.

TABLE 6

| Compound | | |
|---|---|---|
| No. | Intermediate G | Target Compound F (Yield) |
| F73 | 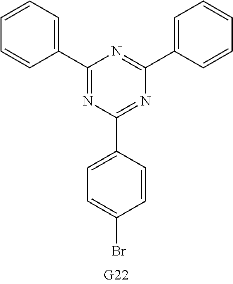 [SMILES: Brc1ccc(-c2nc(-c3ccccc3)nc(-c3ccccc3)n2)cc1] | 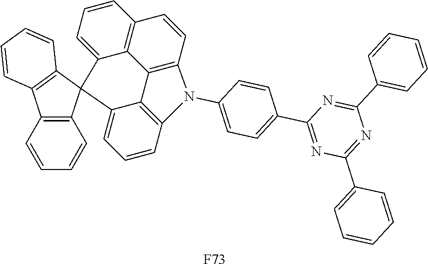 [SMILES: c1ccc(-c2nc(-c3ccccc3)nc(-c3ccc(-n4c5cccc6c5c5c7c(cccc7ccc54)C64c5ccccc5-c5ccccc54)cc3)n2)cc1 ; "F73"] |
| | | (76%) |
| F76 | 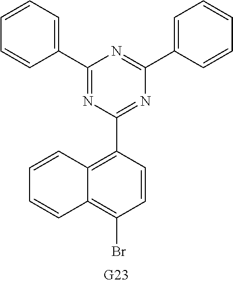 [SMILES: Brc1ccc(-c2nc(-c3ccccc3)nc(-c3ccccc3)n2)c2ccccc12] | 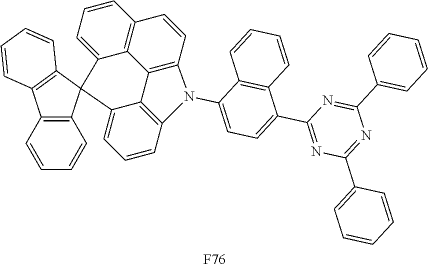 [SMILES: c1ccc(-c2nc(-c3ccccc3)nc(-c3ccc(-n4c5cccc6c5c5c7c(cccc7ccc54)C64c5ccccc5-c5ccccc54)c4ccccc34)n2)cc1 ; "F76"] |
| | | (70%) |
| F81 | 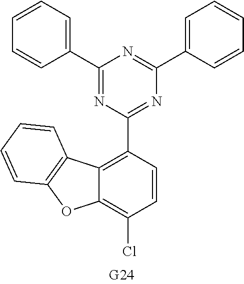 [SMILES: Clc1ccc(-c2nc(-c3ccccc3)nc(-c3ccccc3)n2)c2c1oc1ccccc12] | 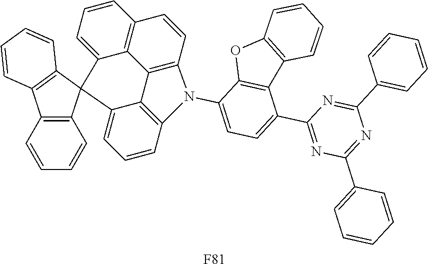 [SMILES: c1ccc(-c2nc(-c3ccccc3)nc(-c3ccc(-n4c5cccc6c5c5c7c(cccc7ccc54)C64c5ccccc5-c5ccccc54)c4oc5ccccc5c34)n2)cc1 ; "F81"] |
| | | (88%) |
| F83 | 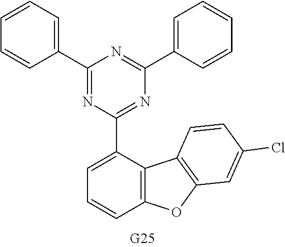 [SMILES: Clc1ccc2c(c1)oc1cccc(-c3nc(-c4ccccc4)nc(-c4ccccc4)n3)c12] | 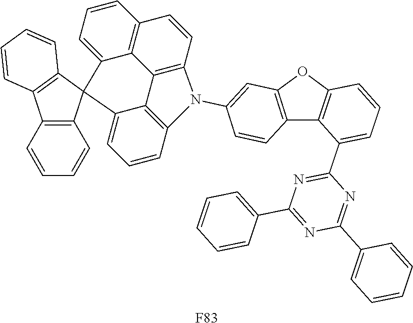 [SMILES: c1ccc(-c2nc(-c3ccccc3)nc(-c3cccc4oc5cc(-n6c7cccc8c7c7c9c(cccc9ccc76)C86c7ccccc7-c7ccccc76)ccc5c34)n2)cc1 ; "F83"] |
| | | (90%) |
| F84 | 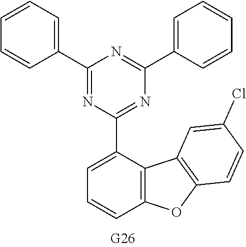 [SMILES: Clc1ccc2oc3cccc(-c4nc(-c5ccccc5)nc(-c5ccccc5)n4)c3c2c1] | 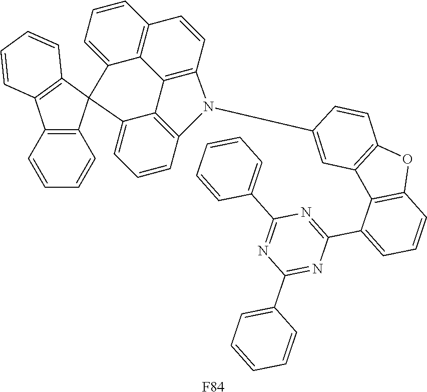 [SMILES: c1ccc(-c2nc(-c3ccccc3)nc(-c3cccc4oc5ccc(-n6c7cccc8c7c7c9c(cccc9ccc76)C86c7ccccc7-c7ccccc76)cc5c34)n2)cc1 ; "F84"] |
| | | (90%) |
| F85 | 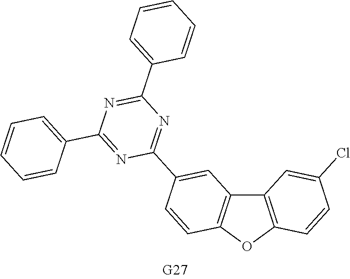 [SMILES: Clc1ccc2oc3ccc(-c4nc(-c5ccccc5)nc(-c5ccccc5)n4)cc3c2c1] | 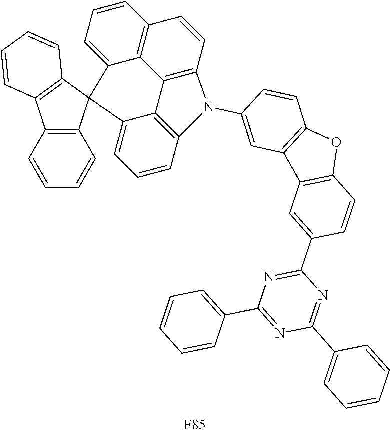 [SMILES: c1ccc(-c2nc(-c3ccccc3)nc(-c3ccc4oc5ccc(-n6c7cccc8c7c7c9c(cccc9ccc76)C86c7ccccc7-c7ccccc76)cc5c4c3)n2)cc1 ; "F85"] |
| | | (93%) |
| F86 | 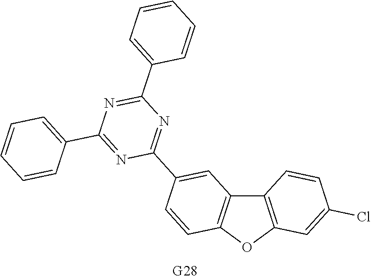 [SMILES: Clc1ccc2c(c1)oc1ccc(-c3nc(-c4ccccc4)nc(-c4ccccc4)n3)cc12] | 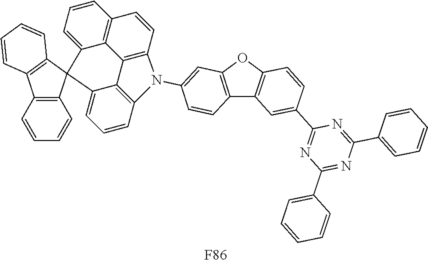 [SMILES: c1ccc(-c2nc(-c3ccccc3)nc(-c3ccc4oc5cc(-n6c7cccc8c7c7c9c(cccc9ccc76)C86c7ccccc7-c7ccccc76)ccc5c4c3)n2)cc1 ; "F86"] |
| | | (91%) |
| F90 | 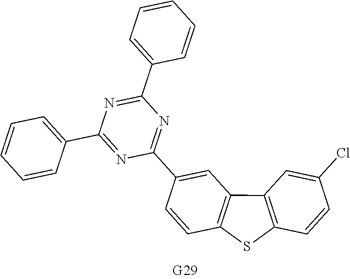 [SMILES: Clc1ccc2sc3ccc(-c4nc(-c5ccccc5)nc(-c5ccccc5)n4)cc3c2c1] | 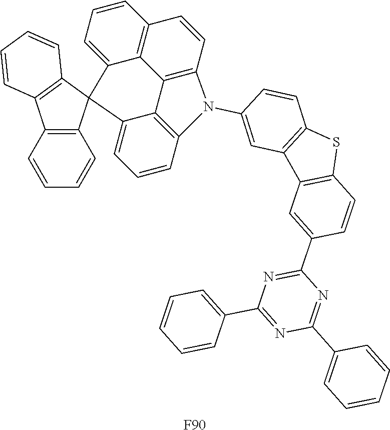 [SMILES: c1ccc(-c2nc(-c3ccccc3)nc(-c3ccc4sc5ccc(-n6c7cccc8c7c7c9c(cccc9ccc76)C86c7ccccc7-c7ccccc76)cc5c4c3)n2)cc1 ; "F90"] |
| | | (93%) |
| F92 | 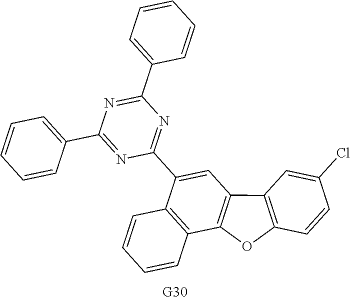 [SMILES: Clc1ccc2oc3c4ccccc4c(-c4nc(-c5ccccc5)nc(-c5ccccc5)n4)cc3c2c1] | 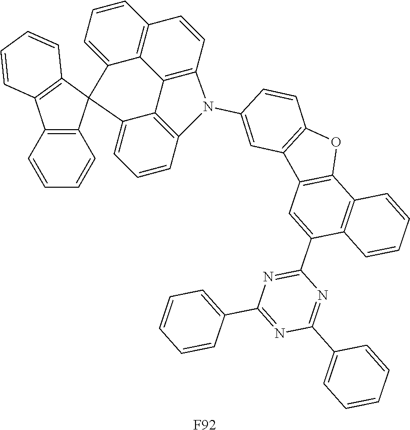 [SMILES: c1ccc(-c2nc(-c3ccccc3)nc(-c3cc4c5cc(-n6c7cccc8c7c7c9c(cccc9ccc76)C86c7ccccc7-c7ccccc76)ccc5oc4c4ccccc34)n2)cc1 ; "F92"] |
| | | (90%) |
| F93 | 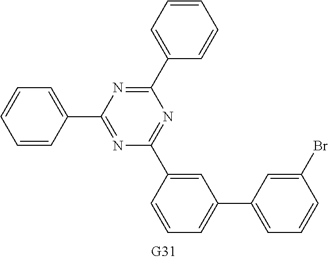 [SMILES: Brc1cccc(-c2cccc(-c3nc(-c4ccccc4)nc(-c4ccccc4)n3)c2)c1] | 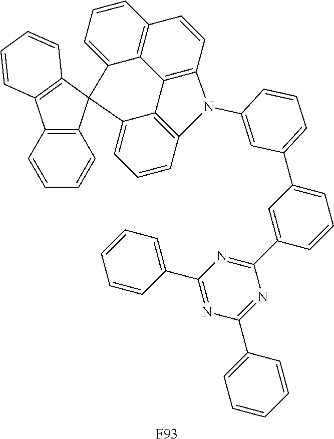 [SMILES: c1ccc(-c2nc(-c3ccccc3)nc(-c3cccc(-c4cccc(-n5c6cccc7c6c6c8c(cccc8ccc65)C75c6ccccc6-c6ccccc65)c4)c3)n2)cc1 ; "F93"] |
| | | (88%) |
| F98 | 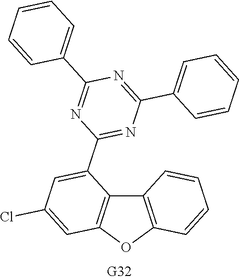 [SMILES: Clc1cc(-c2nc(-c3ccccc3)nc(-c3ccccc3)n2)c2c(c1)oc1ccccc12] | 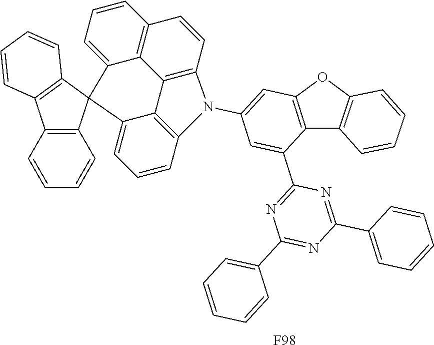 |
| | | (66%) |
| F100 | 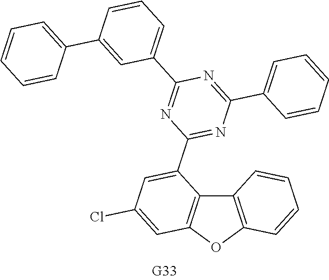 [SMILES: Clc1cc(-c2nc(-c3ccccc3)nc(-c3cccc(-c4ccccc4)c3)n2)c2c(c1)oc1ccccc12] | 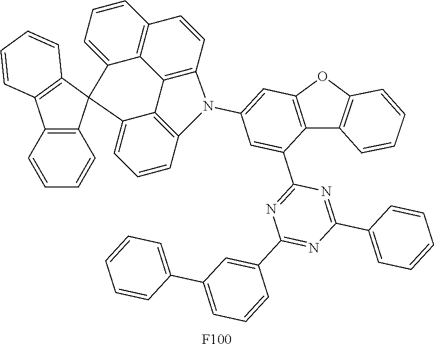 |
| | | (71%) |
| F104 | 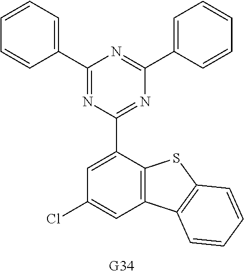 [SMILES: Clc1cc(-c2nc(-c3ccccc3)nc(-c3ccccc3)n2)c2sc3ccccc3c2c1] | 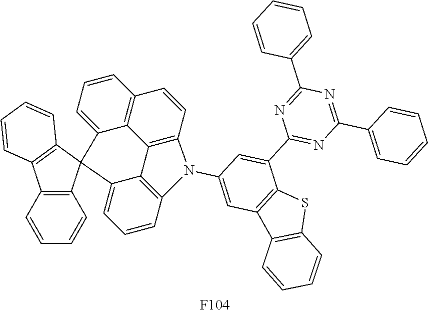 [SMILES: c1ccc(-c2nc(-c3ccccc3)nc(-c3cc(-n4c5cccc6c5c5c7c(cccc7ccc54)C64c5ccccc5-c5ccccc54)cc4c3sc3ccccc34)n2)cc1 ; "F104"] |
| | | (66%) |
| F115 | 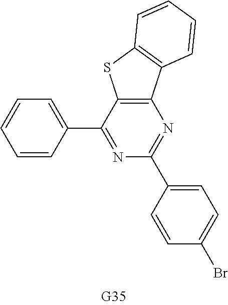 [SMILES: Brc1ccc(-c2nc(-c3ccccc3)c3sc4ccccc4c3n2)cc1] | 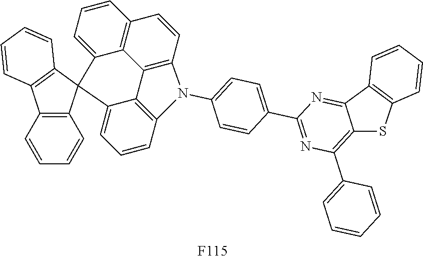 [SMILES: c1ccc(-c2nc(-c3ccc(-n4c5cccc6c5c5c7c(cccc7ccc54)C64c5ccccc5-c5ccccc54)cc3)nc3c2sc2ccccc23)cc1 ; "F115"] |
| | | (51%) |

The remaining Compounds other than the compounds described in Preparative Example 5, Preparative Example 6, Table 5 and Table 6 were prepared in the same manner as in the preparative examples described above, and the synthesis results are shown in Tables 7 and 8 below.

TABLE 7

| Com- | | Com- | |
|---|---|---|---|
| pound | FD-Mass | pound | FD-Mass |
| 1 | m/z = 610.22 (C44H26N4, | 2 | m/z = 583.20 (C43H25N3, |
| | 610.72) | | 583.69) |
| 3 | m/z = 583.20 (C43H25N3, | 4 | m/z = 659.24 (C49H29N3, |
| | 583.69) | | 659.79) |
| 5 | m/z = 686.25 (C50H30N4, | 6 | m/z = 659.24 (C49H29N3, |
| | 686.82) | | 659.79) |
| 7 | m/z = 659.24 (C49H29N3, | 8 | m/z = 623.20 (C45H25N3O, |
| | 659.79) | | 623.72) |
| 9 | m/z = 660.23 (C48H28N4, | 10 | m/ z= 633.22 (C47H27N3, |
| | 660.78) | | 633.75) |
| 11 | m/z = 633.22 (C47H27N3, | 12 | m/z = 639.18 (C45H25N3S, |
| | 633.75) | | 639.78) |
| 13 | m/z = 710.25 (C52H30N4, | 14 | m/z = 633.22 (C47H27N3, |
| | 710.84) | | 633.75) |
| 15 | m/z = 735.27 (C55H33N3, | 16 | m/z = 709.25 (C53H31N3, |
| | 735.89) | | 709.85) |
| 17 | m/z = 736.26 (C54H32N4, | 18 | m/z = 709.25 (C53H31N3, |
| | 736.88) | | 709.85) |
| 19 | m/z = 709.25 (C53H31N3, | 20 | m/z = 673.22 (C49H27N3O, |
| | 709.85) | | 673.78) |
| 21 | m/z = 736.26 (C54H32N4, | 22 | m/z = 709.25 (C53H31N3, |
| | 736.88) | | 709.85) |
| 23 | m/z = 673.22 (C49H27N3O, | 24 | m/z = 639.18 (C45H25N3S, |
| | 673.78) | | 639.78) |
| 25 | m/ z= 620.28 | 26 | m/z = 614.25 (C45H22D5N3, |
| | (C44H16D1ON4, 620.78) | | 614.76) |
| 27 | m/z = 749.25 (C55H31N3O, | 28 | m/z = 673.22 (C49H27N3O, |
| | 749.87) | | 673.78) |
| 29 | m/z = 700.23 (C50H28N4O, | 30 | m/z = 723.23 (C53H29N3O, |
| | 700.80) | | 723.84) |
| 31 | m/z = 723.23 (C53H29N3O, | 32 | m/z = 713.21 |
| | 723.84) | | (C51H27N3O2, 713.80) |
| 33 | m/z = 723.23 (C53H29N3O, | 34 | m/z = 709.25 (C53H31N3, |
| | 723.84) | | 709.85) |
| 35 | m/z = 748.26 (C55H32N4, | 36 | m/z = 790.24 (C56H30N4O2, |
| | 748.89) | | 790.88) |
| 37 | m/z = 716.20 (C50H28N4S, | 38 | m/z = 739.21 (C53H29N3S, |
| | 716.86) | | 739.90) |
| 39 | m/z = 765.22 (C55H31N3S, | 40 | m/z = 660.23 (C48H28N4, |
| | 765.93) | | 660.78) |
| 41 | m/z = 661.23 (C47H27N5, | 42 | m/z = 673.22 (C49H27N3O, |
| | 661.77) | | 673.78) |
| 43 | m/z = 715.21 (C51H29N3S, | 44 | m/ z= 715.21 (C51H29N3S, |
| | 715.87) | | 715.87) |
| 45 | m/z = 726.28 (C53H34N4, | 46 | m/ z= 699.27 (C52H33N3, |
| | 726.88) | | 699.86) |
| 47 | m/z = 684.23 (C50H28N4, | 48 | m/z = 633.22 (C47H27N3, |
| | 684.80) | | 633.75) |
| 49 | m/z = 790.24 | 50 | m/z = 775.27 (C56H33N5, |
| | (C56H30N4O2, 790.88) | | 775.92) |
| 51 | m/z = 775.27 (C56H33N5, | 52 | m/z = 775.27 (C56H33N5, |
| | 775.92) | | 775.92) |
| 53 | m/z = 775.27 (C56H33N5, | 54 | m/z = 622.22 (C45H26N4, |
| | 775.92) | | 622.73) |
| 55 | m/z = 749.26 (C54H31N5, | 56 | m/z = 749.26 (C54H31N5, |
| | 749.88) | | 749.88) |
| 57 | m/z = 776.26 (C56H32N4O, | 58 | m/z = 749.25 |
| | 776.90) | | (C55H31N3O, 749.87) |
| 59 | m/z = 772.26 (C57H32N4, | 60 | m/z = 772.25 (C53H30N4, |
| | 772.91) | | 772.85) |
| 61 | m/z = 790.24 | 62 | m/z = 775.27 (C56H33N5, |
| | (C56H30N4O2, 790.88) | | 775.92) |
| 63 | m/z = 775.27 (C56H33N5, | 64 | m/z = 825.29 (C60H35N5, |
| | 775.92) | | 825.98) |
| 65 | m/z = 709.25 (C53H31N3, | 66 | m/z = 729.19 (C51H27N3OS, |
| | 709.85) | | 729.86) |
| 67 | m/z = 564.15 | 68 | m/z = 729.19 (C51H27N3OS, |
| | (C39H20DN3S, 564.68) | | 729.86) |
| 69 | m/z = 685.25 (C51H31N3, | 70 | m/z = 739.21 (C53H29N3S, |
| | 685.83) | | 739.90) |
| 71 | m/z = 659.24 (C49H29N3, | 72 | m/z = 715.21 (C51H29N3S, |
| | 659.79) | | 715.87) |
| 73 | m/z = 686.25 (C50H30N4, | 74 | m/z = 686.25 (C50H30N4, |
| | 685.82) | | 685.82) |
| 75 | m/z = 685.25 (C51H31N3, | 76 | m/z = 736.26 (C54H32N4, |
| | 685.83) | | 736.88) |
| 77 | m/z = 736.26 (C54H32N4, | 78 | m/z = 736.26 (C54H32N4, |
| | 736.88) | | 736.88) |
| 79 | m/z = 736.26 (C54H32N4, | 80 | m/z = 762.28 (C56H34N4, |
| | 736.88) | | 762.92) |
| 81 | m/z = 776.26 (C56H32N4O, | 82 | m/z = 776.26 (C56H32N4O, |
| | 776.90) | | 776.90) |
| 83 | m/z = 776.26 (C56H32N4O, | 84 | m/z = 776.26 (C56H32N4O, |
| | 776.90) | | 776.90) |
| 85 | m/z = 776.26 (C56H32N4O, | 86 | m/z = 776.26 (C56H32N4O, |
| | 776.90) | | 776.90) |
| 87 | m/z = 776.26 (C56H32N4O, | 88 | m/z = 776.26 (C56H32N4O, |
| | 776.90) | | 776.90) |
| 89 | m/z = 802.31 (C59H38N4, | 90 | m/z = 792.23 (C56H32N4S, |
| | 802.98) | | 792.96) |
| 91 | m/z = 802.31 (C59H38N4, | 92 | m/z = 826.27 (C60H34N4O, |
| | 802.98) | | 826.96) |
| 93 | m/z = 762.28 (C56H34N4, | 94 | m/z = 715.21 (C51H29N3S, |
| | 762.92) | | 715.87) |
| 95 | m/z = 699.23 (C59H29N3O, | 96 | m/z = 659.24 (C49H29N3, |
| | 699.81) | | 659.79) |
| 97 | m/z = 776.26 (C56H32N4O, | 98 | m/z = 776.26 (C56H32N4O, |
| | 776.90) | | 776.90) |
| 99 | m/z = 826.27 (C60H34N4O, | 100 | m/z = 852.29 (C62H36N4O, |
| | 826.96) | | 853.00) |
| 101 | m/z = 802.31 (C59H38N4, | 102 | m/z = 886.27 (C62H34N4O2, |
| | 802.98) | | 886.98) |
| 103 | m/z = 792.23 (C56H32N4S, | 104 | m/z = 792.23 (C56H32N4S, |
| | 792.96) | | 792.96) |
| 105 | m/z = 737.26 (C53H31N5, | 106 | m/z = 709.25 (C53H31N3, |
| | 737.87) | | 709.85) |
| 107 | m/z = 762.28 (C56H34N4, | 108 | m/z = 736.26 (C54H32N4, |
| | 762.92) | | 736.88) |
| 109 | m/z = 533.19 (C39H23N3, | 110 | m/z = 749.25 (C55H31N3O, |
| | 533.63) | | 749.87) |
| 111 | m/z = 805.22 | 112 | m/z = 865.27 (C63H35N3O2, |
| | (C57H31N3OS, 805.96) | | 865.99) |
| 113 | m/z = 659.24 (C49H29N3, | 114 | m/z = 709.25 (C53H31N3, |
| | 659.79) | | 709.85) |
| 115 | m/z = 715.21 (C51H29N3S, | 116 | m/z = 699.23 (C59H29N3O, |
| | 715.87) | | 699.81) |
| 117 | m/z = 687.24 (C49H29N5, | 118 | m/z = 735.27 (C55H33N3, |
| | 687.81) | | 735.89) |
| 119 | m/z = 791.24 (C57H33N3S, | 120 | m/z = 775.26 (C57H33N3O, |
| | 791.97) | | 775.91) |
| 121 | m/z = 686.25 (C50H30N4, | 122 | m/z = 762.28 (C56H34N4, |
| | 686.82) | | 762.92) |
| 123 | m/z = 736.26 (C54H32N4, | 124 | m/z = 775.27 (C56H33N5, |
| | 736.88) | | 775.92) |
| 125 | m/z = 659.24 (C49H29N3, | 126 | m/z = 715.21 (C51H29N3S, |
| | 659.79) | | 715.87) |
| 127 | m/z = 762.28 (C56H34N4, | 128 | m/z = 723.20 (C45H25N3O, |
| | 762.92) | | 723.72) |
| 129 | m/z = 765.22 (C55H31N3S, | 130 | m/z = 765.22 (C55H31N3S, |
| | 765.93) | | 765.93) |
| 131 | m/z = 765.22 (C55H31N3S, | 132 | m/z = 791.24 (C57H33N3S, |
| | 765.93) | | 791.97) |
| 133 | m/z = 761.28 (C57H35N3, | 134 | m/z = 685.25 (C51H31N3, |
| | 761.93) | | 685.83) |
| 135 | m/z = 749.25 (C55H31N3O, | 136 | m/ z= 735.27 (C55H33N3, |
| | 749.87) | | 735.89) |
| 137 | m/z = 659.24 (C49H29N3, | 138 | m/z = 710.25 (C52H30N4, |
| | 659.79) | | 710.84) |
| 139 | m/z = 683.81 (C51H29N3, | 140 | m/z = 735.27 (C55H33N3, |
| | 683.24) | | 735.89) |
| 141 | m/z = 722.34 (C52H42N4, | 142 | m/z = 735.27 (C55H33N3, |
| | 722.94) | | 735.89) |
| 143 | m/z = 715.21 (C51H29N3S, | 144 | m/z = 659.24 (C49H29N3, |
| | 715.87) | | 659.79) |
| 145 | m/z = 791.24 (C57H33N3S, | 146 | m/ z= 735.27 (C55H33N3, |
| | 791.97) | | 735.89) |
| 147 | m/z = 775.26 (C57H33N3O, | 148 | m/z = 791.24 (C57H33N3S, |
| | 775.91) | | 791.97) |
| 149 | m/z = 765.22 (C55H31N3S, | 150 | m/z = 715.21 (C51H29N3S, |
| | 765.93) | | 715.87) |
| 151 | m/z = 791.24 (C57H33N3S, | 152 | m/z = 720.24 (C51H24D5N3S, |
| | 791.97) | | 720.90) |
| 153 | m/z = 791.24 (C57H33N3S, | 154 | m/z = 791.24 (C57H33N3S, |
| | 791.97) | | 791.97) |
| 155 | m/z = 735.27 (C55H33N3, | 156 | m/z = 715.21 (C51H29N3S, |
| | 735.89) | | 715.87) |
| 157 | m/z = 585.20 (C41H23N5, | 158 | m/z = 641.17 (C43H23N5S, |
| | 585.67) | | 641.75) |
| 159 | m/z = 584.20 (C42H24N4, | 160 | m/z = 660.23 (C48H28N4, |
| | 584.68) | | 660.78) |
| 161 | m/z = 659.24 (C49H29N3, | 162 | m/z = 601.20 (C43H24FN3, |
| | 659.79) | | 601.68) |
| 163 | m/z = 749.25 (C55H31N3O, | 164 | m/z = 709.25 (C53H31N3, |
| | 749.87) | | 709.85) |
| 165 | m/z = 824.29 (C61H36N4, | 166 | m/z = 824.29 (C61H36N4, |
| | 824.99) | | 824.99) |
| 167 | m/z = 804.23 (C57H32N4S, | 168 | m/z = 659.24 (C49H29N3, |
| | 804.97) | | 659.79) |
| 169 | m/z = 584.21 (C43H24DN3, | 170 | m/z = 591.26 (C43H17D8N3, |
| | 584.70) | | 591.74) |
| 171 | m/z = 586.22 | 172 | m/z = 588.24 (C43H20D5N3, |
| | (C43H22D3N3, 586.71) | | 588.72) |
| 173 | m/z = 687.27 (C51H33N3, | 174 | m/z = 690.27 (C50H26D4N4, |
| | 687.85) | | 690.84) |
| 175 | m/z = 709.25 (C53H31N3, | 176 | m/z = 709.25 (C53H31N3, |
| | 709.85) | | 709.85) |
| 177 | m/z = 660.21 (C46H24N6, | 178 | m/z = 646.20 (C44H24F2N4, |
| | 660.74) | | 646.70) |
| 179 | m/z = 701.22 (C49H27N5O, | 180 | m/z = 735.27 (C55H33N3, |
| | 701.79) | | 735.89) |

TABLE 8

| Example | ¹H NMR (CDCl₃, 300 Mz) |
|---|---|
| F1 | δ = 9.22(d, 1H), 9.12 (d, 1H), 8.89 (d, 4H), 8.32(d, 1H), |
| | 8.29(d, 1H), 8.23~8.14(m, 4H), 7.62~7.60(m, 4H), 7.59 |
| | (d, 2H), 7.58~7.55(m, 4H), 7.53(d, 1H), 7.19(t, 1H), 7.16 |
| | (t, 1H), 6.92(d, 1H). |
| F2 | δ = 9.16(d, 1H), 8.89(d, 1H), 8.33~8.28(m, 6H), 8.27~8.26 |
| | (m, 4H), 7.99(d, 1H), 7.98~7.96(m, 5H), 7.84(d, 2H), 7.66 |
| | (d, 1H), 7.58(d, 1H), 7.15(t, 1H), 7.14(t, 1H), 6.89(d, 1H). |
| F3 | δ = 9.14(d, 1H), 8.89(d, 1H), 8.33~8.27(m, 11H), 7.97(d, |
| | 1H), 7.96~7.95(m, 4H), 7.83(d, 2H), 7.66(d, 1H), 7.57(d, |
| | 1H), 7.15(t, 1H), 7.14(t, 1H), 6.89(d, 1H). |
| F4 | δ = 9.18 (d, 1H), 8.92(d, 1H), 8.31~8.28(m, 6H), |
| | 8.27~8.26(m, 3H), 8.24(d, 1H), 7.99(d, 1H), 7.97~7.96(m, |
| | 6H), 7.82~7.80(m, 4H), 7.65(d, 1H), 7.58~7.57(m, 3H), |
| | 7.15(t, 1H), 7.11(t, 1H), 6.89(d, 1H). |
| F5 | δ = 9.29(d, 1H), 9.19(d, 1H), 9.29(d, 1H), 8.92~8.91 (m, |
| | 5H), 8.32(d, 1H), 8.29(d, 1H), 8.23~8.14(m, 4H), |
| | 7.62~7.60(m, 4H), 7.59(d, 2H), 7.58~7.56(m, 4H), 7.53(d, |
| | 1H), 7.29~7.27(m, 2H), 7.17(t, 1H), 7.15(t, 1H), 6.96(d, |
| | 1H). |
| F8 | δ = 9.22(d, 1H), 9.16(d, 1H), 8.81 (d, 4H), 8.32(d, 1H), |
| | 8.29(d, 1H), 8.23~8.14(m, 4H), 7.62~7.61(m, 2H), 7.59 |
| | (d, 2H), 7.58~7.57(m, 3H), 7.53(d, 1H), 7.39~7.38(m, 2H), |
| | 7.19(t, 1H), 7.16(t, 1H), 6.92(d, 1H). |
| F9 | δ = 9.22(d, 1H), 9.09(s, 1H), 9.08(d, 2H), 9.10(d, 1H), |
| | 9.09 (d, 1H), 8.32(d, 1H), 8.29(d, 1H), 8.23~8.14(m, 4H), |
| | 7.64(d, 1H), 7.62~7.60(m, 4H), 7.59(d, 2H), 7.58~7.55(m, |
| | 4H), 7.47~7.46(d, 2H), 7.19(t, 1H), 7.16(t, 1H), 6.92(d, |
| | 1H). |
| F12 | δ = 9.22(d, 1H), 9.19(d, 1H), 8.92(d, 1H), 8.89(d, 1H), |
| | 8.32(d, 1H), 8.29(d, 1H), 8.23~8.14(m, 4H), 7.62~7.60(m, |
| | 5H), 7.59(d, 2H), 7.58~7.55(m, 4H), 7.53(d, 1H), 7.19(t, |
| | 1H), 7.16(t, 1H), 6.92(d, 1H). |
| F20 | δ = 9.23(d, 1H), 9.21(s, 1H), 9.16(d, 1H), 8.81 (d, 4H), |
| | 8.32(d, 1H), 8.26(d, 1H), 8.17~8.14(m, 4H), 7.72(s, 1H), |
| | 7.62~7.61(m, 2H), 7.59(d, 2H), 7.58~7.57(m, 3H), 7.53(d, |
| | 1H), 7.39~7.38(m, 2H), 7.20(t, 1H), 7.17(t, 1H), 6.92(d, |
| | 1H). |
| F23 | δ = 9.19(d, 1H), 9.12(d, 1H), 9.11(d, 1H), 8.81 (d, 2H), |
| | 8.32~8.31(m, 2H), 8.29(s, 1H), 8.26~8.24(m, 4H), 7.72(d, |
| | 1H), 7.62~7.61(m, 2H), 7.59(d, 2H), 7.57~7.55(m, 4H), |
| | 7.53(d, 1H), 7.39~7.38(m, 2H), 7.20(t, 1H), 7.17(t, 1H), |
| | 6.89(d, 1H). |
| F24 | δ = 9.24(d, 1H), 9.20(d, 1H), 9.18(d, 1H), 8.81~8.79(m, |
| | 2H), 8.46~8.45(m, 4H), 8.30(s, 1H), 8.26(d, 2H), 7.69(d, |
| | 1H), 7.62~7.61(m, 2H), 7.62(d, 2H), 7.57~7.55(m, 4H), |
| | 7.53(d, 1H), 7.39~7.38(m, 2H), 7.20~7.21(m, 1H), 7.18(t, |
| | 1H), 6.94(d, 1H). |
| F25 | δ = 9.22(d, 1H), 9.12 (d, 1H), 8.32(d, 1H), 8.29(d, 1H), |
| | 8.24~8.23(m, 2H), 7.62~7.60(m, 2H), 7.59(d, 2H), 7.58(d, |
| | 1H), 7.57(d, 1H), 7.53(d, 1H), 7.19(t, 1H), 7.16(t, 1H), |
| | 6.92(d, 1H). |
| F38 | δ = 9.26(d, 1H), 9.13(d, 1H), 8.32~8.29(m, 3H), 8.29(d, |
| | 1H), 8.23~8.22(m, 2H), 8.19(d, 2H), 8.14~8.13(m, 3H), |
| | 7.72(s, 1H), 7.60~7.58(m, 7H), 7.58~7.55(m, 3H), 7.46(d, |
| | 1H), 7.45(d, 1H), 7.21(t, 1H), 7.18(t, 1H), 6.92(d, 1H). |
| F46 | δ = 9.18(d, 1H), 9.17(s, 1H), 8.89(d, 3H), 9.06 (d, 1H), |
| | 8.32(d, 1H), 8.29(d, 1H), 8.22~8.21(m, 2H), 7.73(d, 1H), |
| | 7.60~7.58(m, 4H), 7.57(d, 2H), 7.58~7.55(m, 4H), 7.53(d, |
| | 1H), 7.36~7.35(m, 2H), 7.23(t, 1H), 7.19(t, 1H), 6.92(d, |
| | 1H). 2.14(s, 6H). |
| F48 | δ = 8.98(d, 1H), 8.96(d, 1H), 8.89(d, 1H), 8.66(d, 1H), |
| | 8.65(d, 1H), 8.59(d, 1H), 8.29(d, 1H), 8.28(d, 1H), |
| | 8.24~8.23(m, 2H), 7.59~7.58(m, 5H), 7.57(d, 2H), 7.56(d, |
| | 2H), 7.53(d, 1H), 7.44~7.43(m, 3H), 7.37(t, 1H), 7.19(t, |
| | 1H), 7.16(t, 1H), 6.87(d, 1H). |
| F50 | δ = 9.22(d, 1H), 9.19(d, 1H), 9.12(d, 2H), 8.96(d, 2H), |
| | 8.32(d, 1H), 8.29(d, 1H), 8.26(d, 2H), 8.24~8.23(m, 2H), |
| | 7.62~7.60(m, 4H), 7.59~7.56(m, 6H), 7.53~7.52(m, 5H), |
| | 7.36(t, 1H), 7.34(d, 2H), 7.19(t, 1H), 7.16(t, 1H), 6.92(d, |
| | 1H). |
| F56 | δ = 9.20(d, 1H), 9.18(d, 1H), 9.17(s, 1H), 9.12(d, 2H), |
| | 8.98(d, 2H), 8.34(d, 1H), 8.29(d, 1H), 8.26(d, 1H), |
| | 8.24~8.23(m, 2H), 7.60~7.59(m, 4H), 7.59~7.56(m, 4H), |
| | 7.53~7.52(m, 5H), 7.36(t, 1H), 7.34(d, 2H), 7.19(t, 1H), |
| | 7.16(t, 1H), 6.92(d, 1H). |
| F62 | δ = 9.28(s, 1H), 9.20~9.21(m, 2H), 9.08(d, 1H), 8.98(d, |
| | 2H), 8.32(d, 1H), 8.27(d, 1H), 8.23~8.22(m, 2H), |
| | 7.60~7.58(m, 4H), 7.57(d, 2H), 7.56~7.51(m, 9H), 7.49(d, |
| | 2H), 7.38(t, 1H), 7.20~7.19(m, 3H), 7.16(t, 1H), 6.93(d, |
| | 1H). |
| F63 | δ = 9.20~9.21(m, 2H), 9.14(s, 1H), 9.09(d, 1H), 8.98(d, |
| | 2H), 8.36(d, 1H), 8.32(d, 1H), 8.23~8.22(m, 2H), |
| | 7.59~7.57(m, 4H), 7.56~7.51(m, 11H), 7.45(d, 2H), 7.36 |
| | (t, 1H), 7.20~7.19(m, 3H), 7.16(t, 1H), 6.88(d, 1H). |
| F73 | δ = 9.02(d, 1H), 8.98 (d, 1H), 8.97(d, 4H), 8.89(d, 2H), |
| | 8.32(d, 1H), 8.29(d, 1H), 8.24~8.23(m, 2H), 7.72(d, 2H), |
| | 7.62~7.59(m, 6H), 7.58(d, 2H), 7.57(d, 1H), 7.56(d, 1H), |
| | 7.53(d, 1H), 7.52(d, 2H), 7.19(t, 1H), 7.16(t, 1H), 6.92(d, |
| | 1H). |
| F76 | δ = 9.04 (d, 1H), 8.98 (d, 1H), 8.99(d, 4H), 9.90(d, 1H), |
| | 8.32(d, 1H), 8.29(d, 1H), 8.24~8.23(m, 2H), 7.72(d, 2H), |
| | 7.64(d, 1H), 7.63(d, 1H), 7.62~7.59(m, 5H), 7.59(d, 2H), |
| | 7.58(d, 1H), 7.57(d, 1H), 7.53(d, 1H), 7.52(d, 2H), 7.39(t, |
| | 2H), 7.19(t, 1H), 7.16(t, 1H), 6.92(d, 1H). |
| F81 | δ = 8.99(d, 4H), 8.66(d, 1H), 8.65(d, 1H), 8.44(d, 1H), |
| | 8.39(d, 1H), 8.36(d, 1H), 8.29(d, 1H), 8.16~8.15(m, 2H), |
| | 7.81(d, 1H), 7.56~7.45(m, 12H), 7.40(t, 1H), 7.31(d, 1H), |
| | 7.29~7.27(m, 2H), 7.19(t, 1H), 7.16(t, 1H), 6.89(d, 1H). |
| F83 | δ = 8.8(d, 1H), 8.76(d, 1H), 8.48(d, 4H), 8.27(s, 1H), |
| | 8.19(d, 1H), 7.98~7.89(m, 9H), 7.55~7.47(m, 4H), |
| | 7.41~7.36(m, 5H), 7.24~7.22(m, 6H). |
| F84 | δ = 9.19(s, 1H), 9.07(d, 1H), 9.06(d, 1H), 8.55(d, 1H), |
| | 8.52(d, 4H), 8.21(d, 2H), 8.19(d, 1H), 7.98~7.89(m, 9H), |
| | 7.55~7.47(m, 4H), 7.41~7.38(m, 3H), 7.24~7.21(m, 5H). |
| F85 | δ = 9.26(s, 1H), 9.01(s, 1H), 9.06(d, 1H), 8.56~8.55(m, |
| | 5H), 8.18(d, 1H), 8.14(d, 1H), 7.98~7.86(m, 9H), |
| | 7.55~7.47(m, 4H), 7.41~7.39(m, 3H), 7.24~7.21(m, 6H). |
| F86 | δ = 9.24(s, 1H), 9.01(d, 1H), 8.92(d, 1H), 8.55(d, 4H), |
| | 8.22(d, 2H), 8.14(d, 1H), 7.98~7.88(m, 9H), 7.55~7.46(m, |
| | 4H), 7.41~7.39(m, 3H), 7.22~7.19(m, 6H). |
| F90 | δ = 9.31(s, 1H), 9.19(s, 1H), 9.06(d, 1H), 8.63~8.61(m, |
| | 5H), 8.21(d, 1H), 8.18(d, 1H), 7.98~7.87(m, 9H), |
| | 7.55~7.47(m, 4H), 7.42~7.39(m, 3H), 7.26~7.23(m, 6H). |
| F92 | δ = 9.19(s, 1H), 9.02(d, 1H), 8.74(d, 1H), 8.56(d, 4H), |
| | 8.22(d, 2H), 8.14(d, 1H), 8.06(d, 1H), 7.99~7.90(m, 9H), |
| | 7.56~7.48(m, 4H), 7.41~7.37(m, 6H), 7.22~7.19(m, 4H). |
| F93 | δ = 9.08(s, 1H), 8.93(d, 1H), 8.84(d, 4H), 7.76~7.73(m, |
| | 3H), 7.39~7.28(m, 7H), 7.54~7.47(m, 6H), 7.331(t, 1H), |
| | 7.18(t, 1H), 7.13(t, 1H), 6.98~6.94(m, 4H). |
| F98 | δ = 9.36(s, 1H), 8.98(d, 4H), 8.71(s, 1H), 8.63(d, 1H), |
| | 8.25(d, 1H), 8.20(d, 1H), 7.98~7.86(m, 10H), 7.53~7.46 |
| | (m, 4H), 7.42~7.39(m, 3H), 7.26~7.21(m, 6H). |
| F100 | δ = 9.34(s, 1H), 9.13(d, 1H), 8.98(d, 3H), 8.73(s, 1H), |
| | 8.62(d, 1H), 8.25(d, 1H), 8.20(d, 1H), 7.98~7.84(m, 12H), |
| | 7.53~7.46(m, 6H), 7.43~7.39(m, 3H), 7.29~7.22(m, 6H). |
| F104 | δ = 9.33(s, 1H), 9.28(s, 1H), 8.98(d, 4H), 8.89(d, 1H), |
| | 8.66(d, 1H), 8.27(d, 1H), 8.25(d, 1H), 7.98~7.91(m, 6H), |
| | 7.89~7.83(m, 4H), 7.53~7.46(m, 4H), 7.42~7.36(m, 3H), |
| | 7.31(t, 1H), 7.26~7.21(m, 4H). |
| F115 | δ = 8.59(d, 2H), 8.52(d, 2H), 7.92(d, 1H), 7.90~7.82(m, |
| | 6H), 7.79~7.72(m, 4H), 7.53~7.44(m, 4H), 7.39~7.32(m, |
| | 7H), 6.86~6.84(m, 3H). |
| F121 | δ = 9.27(s, 1H), 9.12 (d, 1H), 8.88 (d, 4H), 8.27(d, 1H), |
| | 8.21~8.13(m, 6H), 7.62~7.58(m, 4H), 7.59(d, 2H), |
| | 7.58~7.54(m, 6H), 7.21~7.20(m, 3H), 7.18(t, 1H), 6.92(d, |
| | 1H). |
| F125 | δ = 9.18(s, 1H), 8.92(d, 1H), 8.33~8.27(m, 5H), 8.27~8.26 |
| | (m, 4H), 7.97(d, 1H), 7.98~7.94(m, 6H), 7.86(d, 2H), |
| | 7.66~7.63(m, 2H), 7.59(d, 1H), 7.15~7.11(m, 5H), 6.89(d, |
| | 1H). |
| F132 | δ = 9.27(s, 1H), 9.19(d, 1H), 8.89(d, 2H), 8.34(d, 1H), |
| | 8.32(d, 1H), 8.24~8.16(m, 4H), 7.67~7.59(m, 7H), 7.59 |
| | (d, 2H), 7.58~7.55(m, 8H), 7.53(d, 1H), 7.19(t, 1H), |
| | 7.17~7.14(m, 3H), 6.92(d, 1H). |
| F135 | δ = 9.23(s, 1H), 8.98(d, 1H), 8.42(s, 1H), 8.33~8.28(m, |
| | 6H), 8.27~8.26(m, 4H), 7.99(d, 1H), 7.98~7.88(d, 6H), |
| | 7.66~7.59(m, 3H), 7.58~7.52(m, 3H), 7.15~7.12(m, 4H), |
| | 6.92(d, 1H). |
| F136 | δ = 9.17(d, 1H), 8.89(d, 1H), 8.33~8.28(m, 6H), 8.27~8.26 |
| | (m, 4H), 7.99(d, 1H), 7.98~7.92(m, 7H), 7.84(d, 2H), 7.66 |
| | (d, 1H), 7.58(d, 1H), 7.24(s, 2H), 7.21~7.14(m, 6H), 6.81 |
| | (d, 1H). |
| F139 | δ = 9.27(d, 1H), 9.16(d, 1H), 8.52(d, 2H), 8.33~8.28(m, |
| | 6H), 8.27~8.26(m, 4H), 7.99(d, 1H), 7.98~7.96(m, 5H), |
| | 7.84(d, 2H), 7.66(d, 1H), 7.58(d, 1H), 7.23~7.14(m, 3H), |
| | 6.89(d, 1H). |
| F140 | δ = 9.16(d, 1H), 8.89(d, 1H), 8.33~8.28(m, 6H), 8.27~8.26 |
| | (m, 4H), 7.99(d, 1H), 7.98~7.92(m, 7H), 7.88(d, 2H), |
| | 7.63~7.59(m, 3H), 7.52(d, 1H), 7.24~7.15(m, 6H), 6.89(d, |
| | 1H). |
| F141 | δ = 9.22(d, 1H), 9.12 (d, 1H), 8.89 (d, 4H), 8.32(d, 1H), |
| | 8.29(d, 1H), 8.23~8.14(m, 4H), 7.62~7.59(m, 4H), 7.84(d, |
| | 2H), 7.58(d, 1H), 7.57~7.53(m, 3H), 7.24(t, 1H), 7.20(t, |
| | 1H), 1.59(s, 18H). |
| F143 | δ = 9.18 (d, 1H), 9.16(d, 1H), 8.92(d, 1H), 8.89(d, 1H), |
| | 8.32(d, 1H), 8.29(d, 1H), 8.23~8.14(m, 4H), 7.63~7.58(m, |
| | 5H), 7.58(d, 2H), 7.57~7.52(m, 6H), 7.53(d, 1H), |
| | 7.21~7.18(m, 3H), 7.16(t, 1H), 6.92(d, 1H). |
| F152 | δ = 9.18 (d, 1H), 9.16(d, 1H), 8.92(d, 1H), 8.89(d, 1H), |
| | 8.32(d, 1H), 8.29(d, 1H), 8.23~8.14(m, 4H), 7.63~7.58(m, |
| | 3H), 7.58(d, 2H), 7.57~7.52(m, 4H), 7.53(d, 1H), |
| | 7.21~7.18(m, 2H), 7.16(t, 1H), 6.92(d, 1H). |
| F157 | δ = 9.17(d, 1H), 9.15(d, 1H), 8.38(d, 2H), 8.36~8.33(m, |
| | 3H), 7.96~7.87(m, 6H), 7.64~7.59(m, 6H), 7.26~7.23(m, |
| | 3H), 7.19(t, 1H). |
| F158 | δ = 9.24(d, 1H), 9.18(d, 1H), 8.39(d, 2H), 8.34(d, 1H), |
| | 7.96~7.85(m, 8H), 7.64~7.61(m, 6H), 7.26~7.21(m, 3H), |
| | 7.19(t, 1H). |
| F159 | δ = 9.29(s, 1H), 9.15(d, 1H), 8.36~8.33(m, 4H), 7.96~7.87 |
| | (m, 7H), 7.68(d, 1H), 7.61~7.57(m, 5H), 7.29~7.23(m, 3H), |
| | 7.19(t, 1H). |
| F166 | δ = 9.33(s, 1H), 8.89(d, 1H), 8.82(s, 1H), 8.33~8.28(m, |
| | 6H), 8.27~8.23(m, 4H), 7.99(d, 1H), 7.98~7.92(m, 5H), |
| | 7.81(d, 2H), 7.65~7.58(d, 6H), 7.27~7.14(m, 6H), 7.09(d, |
| | 2H, 6.89(d, 1H). |
| F168 | δ = 9.13(d, 1H), 8.87(d, 1H), 8.34~8.28(m, 5H), 8.27~8.23 |
| | (m, 2H), 8.00(d, 1H), 7.98~7.93(m, 6H), 7.84(d, 2H), 7.66 |
| | (d, 1H), 7.58~7.53(m, 5H), 7.18~7.14(m, 4H), 6.86(d, 1H). |
| F170 | δ = 9.16(d, 1H), 8.33~8.28(m, 4H), 8.27(d, 1H), 8.26(t, |
| | 1H), 7.99(d, 1H), 7.98(d, 1H), 7.84(d, 2H), 7.66(d, 1H), |
| | 7.58-7.56(m, 2H), 7.15(t, 1H), 7.14(t, 1H), 6.86(d, 1H). |
| F171 | δ = 9.16(d, 1H), 8.89(d, 1H), 8.33~8.28(m, 6H), 8.27~8.26 |
| | (m, 2H), 7.99(d, 1H), 7.98~7.96(m, 4H), 7.84(d, 2H), 7.66 |
| | (d, 1H), 7.58(d, 1H), 7.15(t, 1H), 7.14(t, 1H), 6.89(d, 1H). |
| E177 | δ = 9.26(d, 1H), 9.18(d, 1H), 9.07(d, 4H), 8.33(d, 1H), |
| | 8.29(d, 1H), 8.23~8.14(m, 4H), 7.66(d, 2H), 7.63~7.61(m, |
| | 2H), 7.59(d, 2H), 7.58~7.55(m, 2H), 7.53(d, 1H), 7.19(t, |
| | 1H), 7.17(t, 1H), 6.94(d, 1H). |
| F178 | δ = 9.23(d, 1H), 9.16(d, 1H), 9.01(d, 4H), 8.32(d, 1H), |
| | 8.28(d, 1H), 8.23~8.16(m, 4H), 7.62~7.58(m, 6H), |
| | 7.53~7.48(m, 3H), 7.19(t, 1H), 7.16(t, 1H), 6.92(d, 1H). |

### Experimental Example 1

(1) Manufacturing of Organic Light-Emitting Devices

A glass substrate coated with a thin film of indium tin oxide (ITO) to a thickness of 1500 Å was washed with distilled water ultrasonic waves. After washing with distilled water, it was ultrasonically washed with a solvent such as acetone, methanol, isopropyl alcohol, and the like, and dried, and then treated with ultraviolet ozone (UVO) for 5 minutes using UV in a UV cleaner. Thereafter, the substrate was transferred to a plasma cleaner (PT), and then plasma-treated in a vacuum to increase the work function of ITO and remove the residual film, and transferred to a thermal deposition equipment for organic deposition.

2-TNATA (4,4′,4″-tris[2-naphthyl(phenyl)amino]triphenylamine) as a hole injection layer and NPB (N,N′-di(1-naphthyl)-N,N′-diphenyl-(1,1′-biphenyl)-4,4′-diamine) as a hole transport layer were formed on the ITO transparent electrode (anode), which is a common layer, respectively.

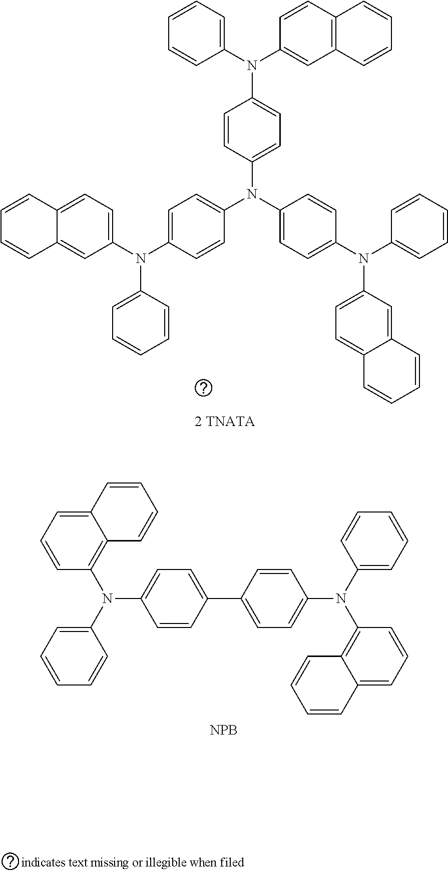 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light-emitting layer was thermally vacuum deposited thereon as follows. The light-emitting layer was formed by depositing the compound described in Table 9 below as a red host, and doping (piq)₂(Ir) (acac) to the host at 3 wt % using (piq)₂(Ir) (acac) as a red phosphorescent dopant and depositing it to a thickness of 400 Å. Thereafter, bathophenanthroline (Phen) was deposited to a thickness of 30 Å as a hole-blocking layer, and Alqⱼ was deposited to a thickness of 250 Å thereon as an electron transport layer. Finally, lithium fluoride (LiF) was deposited to a thickness of 10 Å on the electron transport layer to form an electron injection layer, and then an aluminum (Al) was deposited to a thickness of 1,200 Å on the electron injection layer to form a cathode, thereby manufacturing an organic light-emitting device.

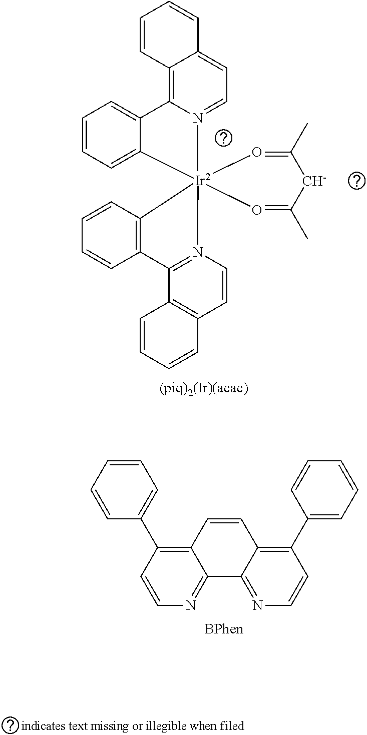

On the other hand, all organic compounds required for manufacturing OLED devices were purified by vacuum sublimation under 10⁻⁴ to 10⁻⁸ torr for each material, and used for manufacturing an organic light-emitting diode (TIED) device.

(2) Driving Voltage and Luminous Efficiency of Organic Light-Emitting Devices

For the organic light-emitting device manufactured as described above, electroluminescence (EL) properties were measured with M7000 from McScience Inc., and based on the measured results, T₉₀ was measured when the reference luminance was 6,000 cd/m² through the lifespan measuring device (M6000) manufactured by McScience Inc. The properties of the organic light-emitting device of the present invention are shown in Table 9 below. In the above, T₉₀ means a lifespan (unit: time (h)) that is a time at which the luminance become 90% relative to the initial luminance.

The results of measuring the driving voltage, luminous efficiency, color coordinates (CIE), and lifespan of the organic light-emitting devices manufactured according to the present invention are shown in Table 9 below. In addition, the results of measuring the driving voltage, luminous efficiency, color coordinates (CIE), and lifespan of the organic light-emitting devices manufactured using the following comparative compound as a compound for the light-emitting layer are shown in Table 9 below.

TABLE 9

| | | Driving | | Color | |
|---|---|---|---|---|---|
| | Com- | Voltage | Efficiency | coordinates | lifespan |
| | pound | (V) | (cd/A) | (x, y) | (T₉₀ ) |
| Example 1 | F1 | 4.01 | 32.7 | (0.683, | 229 |
| | | | | 0.317) | |
| Example 2 | F2 | 3.87 | 33.6 | (0.683, | 280 |
| | | | | 0.317) | |
| Example 3 | F3 | 3.79 | 33.6 | (0.683, | 275 |
| | | | | 0.317) | |
| Example 4 | F4 | 4.14 | 32.6 | (0.683, | 277 |
| | | | | 0.317) | |
| Example 5 | F5 | 3.87 | 33.9 | (0.683, | 242 |
| | | | | 0.317) | |
| Example 6 | F8 | 3.79 | 33.6 | (0.683, | 190 |
| | | | | 0.317) | |
| Example 7 | F9 | 3.59 | 33.6 | (0.683, | 268 |
| | | | | 0.317) | |
| Example 8 | F12 | 4.06 | 30.6 | (0.683, | 320 |
| | | | | 0.317) | |
| Example 9 | F20 | 3.99 | 34.6 | (0.683, | 155 |
| | | | | 0.317) | |
| Example 10 | F23 | 3.88 | 29.6 | (0.683, | 148 |
| | | | | 0.317) | |
| Example 11 | F24 | 3.99 | 31.6 | (0.684, | 340 |
| | | | | 0.316) | |
| Example 12 | F25 | 3.99 | 32.8 | (0.683, | 323 |
| | | | | 0.317) | |
| Example 13 | F38 | 3.78 | 29.6 | (0.683, | 298 |
| | | | | 0.317) | |
| Example 14 | F46 | 4.11 | 35.6 | (0.683, | 192 |
| | | | | 0.317) | |
| Example 15 | F48 | 4.42 | 29.6 | (0.683, | 112 |
| | | | | 0.317) | |
| Example 16 | F50 | 3.92 | 36.6 | (0.683, | 190 |
| | | | | 0.317) | |
| Example 17 | F56 | 3.80 | 30.6 | (0.683, | 198 |
| | | | | 0.317) | |
| Example 18 | F62 | 4.12 | 34.6 | (0.683, | 222 |
| | | | | 0.317) | |
| Example 19 | F63 | 4.22 | 36.6 | (0.683, | 218 |
| | | | | 0.317) | |
| Example 20 | F73 | 4.21 | 36.6 | (0.682, | 167 |
| | | | | 0.318) | |
| Example 21 | F76 | 4.19 | 36.6 | (0.683, | 170 |
| | | | | 0.317) | |
| Example 22 | F81 | 3.91 | 36.2 | (0.683, | 200 |
| | | | | 0.317) | |
| Example 23 | F83 | 3.98 | 36.6 | (0.683, | 242 |
| | | | | 0.317) | |
| Example 24 | F84 | 4.19 | 35.2 | (0.683, | 288 |
| | | | | 0.317) | |
| Example 25 | F85 | 3.82 | 37.6 | (0.683, | 176 |
| | | | | 0.317) | |
| Example 26 | F86 | 3.86 | 35.6 | (0.683, | 184 |
| | | | | 0.317) | |
| Example 27 | F90 | 3.79 | 31.6 | (0.683, | 244 |
| | | | | 0.317) | |
| Example 28 | F92 | 4.14 | 33.6 | (0.683, | 187 |
| | | | | 0.317) | |
| Example 29 | F93 | 4.27 | 33.6 | (0.683, | 156 |
| | | | | 0.317) | |
| Example 30 | F98 | 3.75 | 33.6 | (0.683, | 343 |
| | | | | 0.317) | |
| Example 31 | F100 | 3.78 | 32.6 | (0.683, | 325 |
| | | | | 0.317) | |
| Example 32 | F104 | 4.11 | 32.6 | (0.683, | 259 |
| | | | | 0.317) | |
| Example 33 | F115 | 4.21 | 33.6 | (0.683, | 285 |
| | | | | 0.317) | |
| Example 34 | F121 | 3.89 | 33.6 | (0.683, | 254 |
| | | | | 0.317) | |
| Example 35 | F125 | 4.11 | 34.6 | (0.683, | 296 |
| | | | | 0.317) | |
| Example 36 | F132 | 3.82 | 34.6 | (0.683, | 309 |
| | | | | 0.317) | |
| Example 37 | F135 | 3.88 | 31.9 | (0.683, | 258 |
| | | | | 0.317) | |
| Example 38 | F136 | 4.08 | 33.9 | (0.681, | 253 |
| | | | | 0.318) | |
| Example 39 | F139 | 4.15 | 36.5 | (0.682, | 302 |
| | | | | 0.316) | |
| Example 40 | F140 | 4.08 | 36.9 | (0.681, | 308 |
| | | | | 0.318) | |
| Example 41 | F141 | 4.08 | 39.8 | (0.681, | 161 |
| | | | | 0.318) | |
| Example 42 | F143 | 4.08 | 34.7 | (0.681, | 288 |
| | | | | 0.318) | |
| Example 43 | F148 | 4.08 | 33.3 | (0.682, | 176 |
| | | | | 0.316) | |
| Example 44 | F152 | 4.02 | 33.9 | (0.681, | 282 |
| | | | | 0.318) | |
| Example 45 | F157 | 4.12 | 29.2 | (0.681, | 189 |
| | | | | 0.318) | |
| Example 46 | F158 | 4.12 | 32.1 | (0.681, | 221 |
| | | | | 0.318) | |
| Example 47 | F159 | 4.02 | 33.9 | (0.682, | 238 |
| | | | | 0.316) | |
| Example 48 | F166 | 4.12 | 28.9 | (0.681, | 176 |
| | | | | 0.318) | |
| Example 49 | F168 | 4.02 | 37.0 | (0.681, | 220 |
| | | | | 0.318) | |
| Example 50 | F170 | 4.02 | 34.6 | (0.681, | 338 |
| | | | | 0.318) | |
| Example 51 | F171 | 4.02 | 33.9 | (0.682, | 330 |
| | | | | 0.318) | |
| Example 52 | F177 | 4.12 | 37.6 | (0.683, | 138 |
| | | | | 0.317) | |
| Example 53 | F178 | 4.12 | 36.6 | (0.682, | 166 |
| | | | | 0.316) | |
| Comparative | A | 5.24 | 9.4 | (0.682, | 44 |
| Example 1 | | | | 0.318) | |
| Comparative | B | 5.09 | 11.2 | (0.682, | 89 |
| Example 2 | | | | 0.318) | |
| Comparative | C | 5.19 | 9.5 | (0.684, | 85 |
| Example 3 | | | | 0.316) | |
| Comparative | D | 4.82 | 24.2 | (0.683, | 39 |
| Example 4 | | | | 0.317) | |
| Comparative | E | 4.76 | 23.3 | (0.682, | 128 |
| Example 5 | | | | 0.318) | |
| Comparative | F | 4.88 | 19.9 | (0.683, | 129 |
| Example 6 | | | | 0.317) | |
| Comparative | G | 4.58 | 22.1 | (0.684, | 112 |
| Example 7 | | | | 0.316) | |
| Comparative | H | 4.69 | 18.9 | (0.683, | 57 |
| Example 8 | | | | 0.317) | |
| Comparative | I | 5.18 | 17.9 | (0.683, | 63 |
| Example 9 | | | | 0.317) | |

### [Comparative Compounds A˜I]

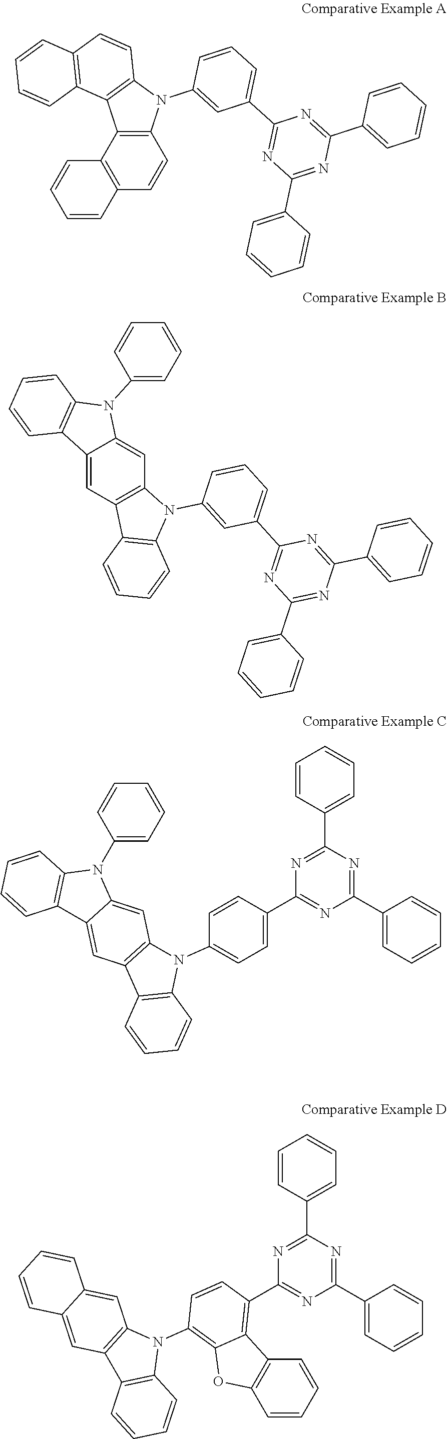 [SMILES: c1ccc(-c2nc(-c3ccccc3)nc(-c3cccc(-n4c5ccc6ccccc6c5c5c6ccccc6ccc54)c3)n2)cc1 ; c1ccc(-c2nc(-c3ccccc3)nc(-c3cccc(-n4c5ccccc5c5cc6c7ccccc7n(-c7ccccc7)c6cc54)c3)n2)cc1 ; c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2nc(-c3ccccc3)nc(-c3ccc(-n4c5ccccc5c5cc6ccccc6cc54)c4oc5ccccc5c34)n2)cc1]

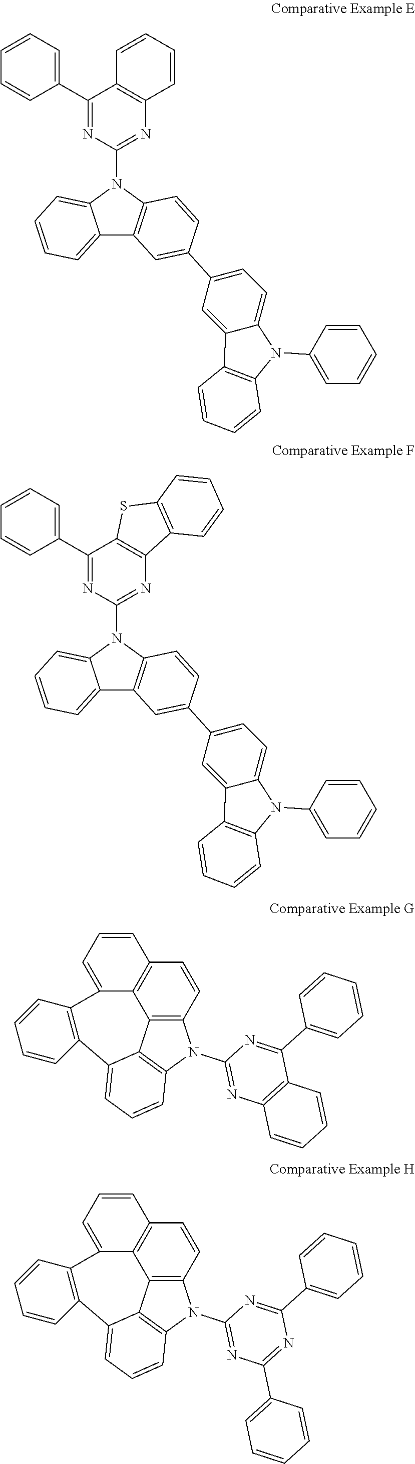 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-c2nc(-n3c4ccccc4c4cc(-c5ccc6c(c5)c5ccccc5n6-c5ccccc5)ccc43)nc3c2sc2ccccc23)cc1 ; c1ccc(-c2nc(-n3c4cccc5c4c4c6c(cccc6ccc43)-c3ccccc3-5)nc3ccccc23)cc1 ; c1ccc(-c2nc(-c3ccccc3)nc(-n3c4cccc5c4c4c6c(cccc6ccc43)-c3ccccc3-5)n2)cc1]

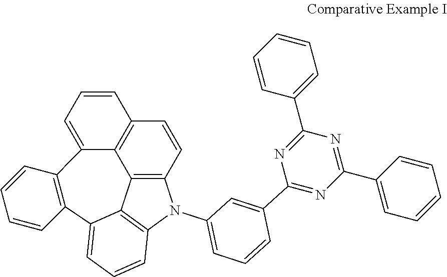 [SMILES: c1ccc(-c2nc(-c3ccccc3)nc(-c3cccc(-n4c5cccc6c5c5c7c(cccc7ccc54)-c4ccccc4-6)c3)n2)cc1]

Looking at the results of Table 9 above, when the heterocyclic compound represented by Formula 1 is used as a light-emitting layer of the organic light-emitting device, it could be confirmed to exhibit an excellent effect in lifespan, luminous efficiency, and driving voltage properties compared to the case of using Comparative Compounds A to I.

In particular, it could be confirmed that the compound represented by Formula 1 improved hole injection and hole transport properties by substituting a cyclized fluorene group under the naphthocarbazole structure, thereby providing an appropriate energy level and thermal stability to the organic light-emitting device, and an organic light-emitting device with improved lifespan, driving stability and efficiency may be manufactured by using the compound represented by Formula 1.

### Experimental Example 2

(1) Manufacturing of Organic Light-Emitting Devices

A glass substrate coated with a thin film of indium tin oxide (ITO) to a thickness of 1500 Å was washed with distilled water ultrasonic waves. After washing with distilled water, it was ultrasonically washed with a solvent such as acetone, methanol, isopropyl alcohol, and the like, and dried, and then treated with ultraviolet ozone (UVO) for 5 minutes using UV in a UV cleaner. Thereafter, the substrate was transferred to a plasma cleaner (PT), and then plasma-treated in a vacuum to increase the work function of ITO and remove the residual film, and transferred to a thermal deposition equipment for organic deposition.

2-TNATA (4,4′,4″-tris[2-naphthyl(phenyl)amino]triphenylamine) as a hole injection layer, NPB (N,N′-di(1-naphthyl)-N,N′-diphenyl-(1,1′-biphenyl)-4,4′-diamine) as a hole transport layer, and cyclohexylidenebis[N,N-bis(4-methylphenyl)benzenamine] (TAPC) as an electron-blocking layer or tris(4-carbazoyl-9-ylphenyl)amine (TCTA) as an exciton-blocking layer were formed on the ITO transparent electrode (anode), which is a common layer, respectively.

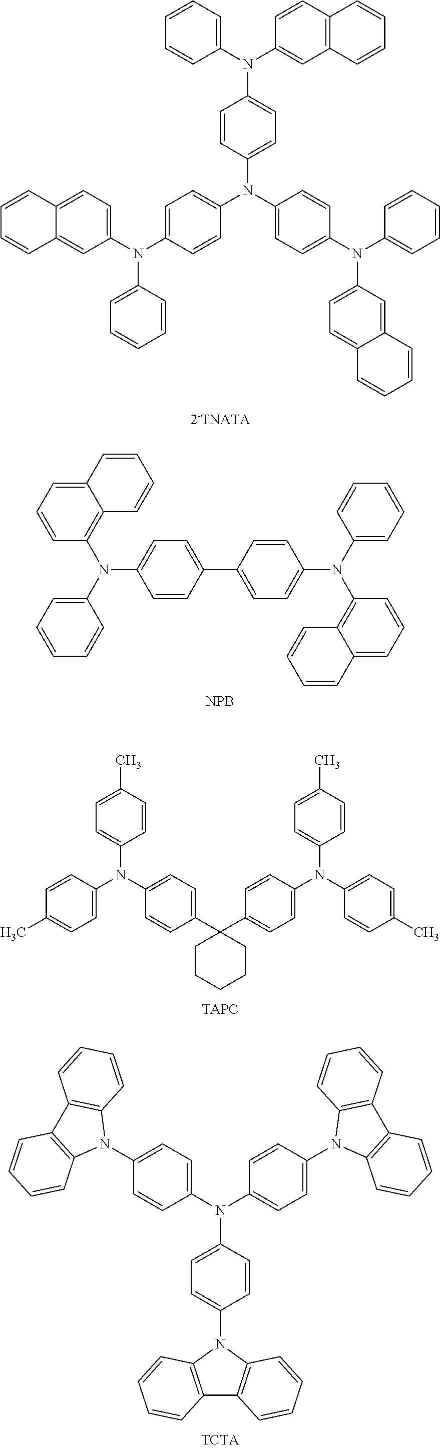

A light-emitting layer was thermally vacuum deposited thereon as follows. The light-emitting layer was formed by depositing the compound described in Table 10 below as a red host, and doping (piq)₂(Ir) (acac) to the host at 3 wt % using (piq)₂(Ir) (acac) as a red phosphorescent dopant and depositing it to a thickness of 400 Å. Thereafter, bathophenanthroline (BPhen) was deposited to a thickness of 30 Å as a hole-blocking layer, and 2,2′,2″-(1,3,5-Benzenetriyl)-tris(1-phenyl-1-H-benzimidazole) (TPBI) was deposited to a thickness of 250 Å thereon as an electron transport layer. Finally, lithium fluoride (LiF) was deposited to a thickness of 10 Å on the electron transport layer to form an electron injection layer, and then an aluminum (Al) as deposited to a thickness of 1,200 Å on the electron injection layer to form a cathode, thereby manufacturing an organic light-emitting device.

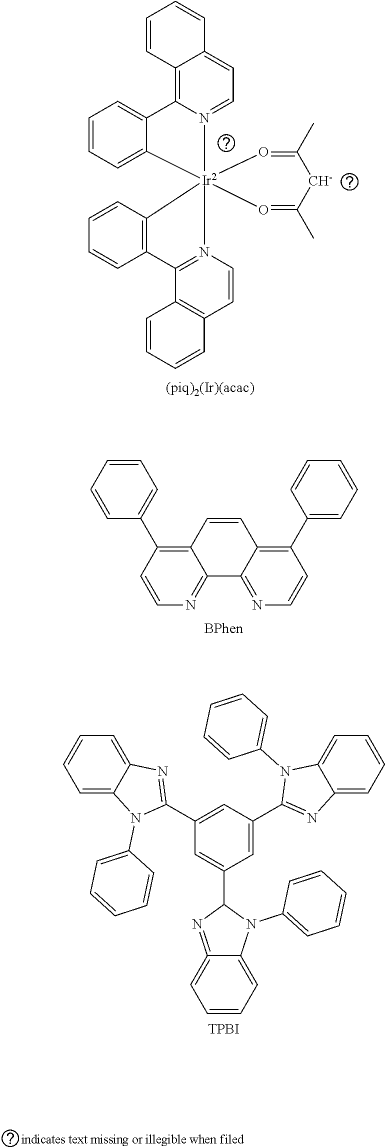

On the other hand, all organic compounds required for manufacturing OLED devices were purified by vacuum sublimation under 10⁻⁸ to 10⁻⁶ torr for each material, and used for manufacturing an organic light-emitting diode (OLED) device.

(2) Driving Voltage and Luminous Efficiency of Organic Light-Emitting Devices

For the organic light-emitting device manufactured as described above, electroluminescence (EL) properties were measured with M7000 from McScience Inc., and based on the measured results, T₉₀ was measured when the reference luminance was 6,000 cd/m² through the lifespan measuring device (M6000) manufactured by McScience Inc. The properties of the organic light-emitting device of the present invention are shown in Table 10 below. In the above, T₉₀ means a lifespan (unit: time (h)) that is a time at which the luminance become 90% relative to the initial luminance.

The results of measuring the driving voltage, luminous efficiency, color coordinates (CIE), and lifespan of the organic light-emitting devices manufactured according to the present invention are shown in Table 10 below. In addition, the results of measuring the driving voltage, luminous efficiency, color coordinates (CIE), and lifespan of the organic light-emitting devices manufactured using the following comparative compound as a compound for the light-emitting layer are shown in Table 10 below.

TABLE 10

| | | Driving | | Color | |
|---|---|---|---|---|---|
| | Com- | Voltage | Efficiency | coordinates | lifespan |
| | pound | (V) | (cd/A) | (x, y) | (T₉₀) |
| Example 54 | F1 | 4.02 | 39.7 | (0.683, | 209 |
| | | | | 0.317) | |
| Example 55 | F2 | 3.89 | 45.6 | (0.683, | 300 |
| | | | | 0.317) | |
| Example 56 | F3 | 3.81 | 43.6 | (0.683, | 325 |
| | | | | 0.317) | |
| Example 57 | F4 | 4.14 | 42.6 | (0.683, | 297 |
| | | | | 0.317) | |
| Example 58 | F5 | 3.97 | 40.9 | (0.683, | 282 |
| | | | | 0.317) | |
| Example 59 | F8 | 3.81 | 42.6 | (0.683, | 210 |
| | | | | 0.317) | |
| Example 60 | F9 | 3.64 | 39.6 | (0.683, | 288 |
| | | | | 0.317) | |
| Example 61 | F12 | 4.08 | 38.6 | (0.683, | 360 |
| | | | | 0.317) | |
| Example 62 | F25 | 3.99 | 39.8 | (0.683, | 383 |
| | | | | 0.317) | |
| Example 63 | F50 | 3.98 | 43.6 | (0.683, | 210 |
| | | | | 0.317) | |
| Example 64 | F62 | 4.19 | 44.6 | (0.683, | 242 |
| | | | | 0.317) | |
| Example 65 | F63 | 4.32 | 42.6 | (0.683, | 288 |
| | | | | 0.317) | |
| Example 66 | F73 | 4.31 | 42.6 | (0.682, | 177 |
| | | | | 0.318) | |
| Example 67 | F76 | 4.21 | 43.6 | (0.683, | 178 |
| | | | | 0.317) | |
| Example 68 | F81 | 3.99 | 43.2 | (0.683, | 220 |
| | | | | 0.317) | |
| Example 69 | F83 | 3.98 | 42.6 | (0.683, | 282 |
| | | | | 0.317) | |
| Example 70 | F84 | 4.18 | 42.2 | (0.683, | 296 |
| | | | | 0.317) | |
| Example 71 | F85 | 3.93 | 44.6 | (0.683, | 180 |
| | | | | 0.317) | |
| Example 72 | F86 | 3.92 | 38.6 | (0.683, | 190 |
| | | | | 0.317) | |
| Example 73 | F90 | 3.91 | 39.6 | (0.683, | 256 |
| | | | | 0.317) | |
| Example 74 | F92 | 4.20 | 38.6 | (0.683, | 194 |
| | | | | 0.317) | |
| Example 75 | F93 | 4.33 | 39.6 | (0.683, | 170 |
| | | | | 0.317) | |
| Example 76 | F98 | 3.79 | 43.6 | (0.683, | 340 |
| | | | | 0.317) | |
| Example 77 | F100 | 3.78 | 44.6 | (0.683, | 348 |
| | | | | 0.317) | |
| Example 78 | F104 | 4.18 | 40.6 | (0.683, | 320 |
| | | | | 0.317) | |
| Example 79 | F115 | 4.31 | 40.6 | (0.683, | 330 |
| | | | | 0.317) | |
| Example 80 | F121 | 3.90 | 41.6 | (0.683, | 280 |
| | | | | 0.317) | |
| Example 81 | F125 | 4.21 | 43.6 | (0.683, | 317 |
| | | | | 0.317) | |
| Example 82 | F132 | 3.87 | 43.6 | (0.683, | 329 |
| | | | | 0.317) | |
| Example 83 | F135 | 3.89 | 42.9 | (0.683, | 308 |
| | | | | 0.317) | |
| Example 84 | F136 | 4.18 | 40.9 | (0.681, | 303 |
| | | | | 0.318) | |
| Example 85 | F139 | 4.25 | 43.5 | (0.682, | 312 |
| | | | | 0.316) | |
| Example 86 | F140 | 4.18 | 43.9 | (0.681, | 318 |
| | | | | 0.318) | |
| Example 87 | F141 | 4.18 | 44.8 | (0.681, | 159 |
| | | | | 0.318) | |
| Example 88 | F143 | 4.18 | 39.7 | (0.681, | 280 |
| | | | | 0.318) | |
| Example 89 | F148 | 4.18 | 39.3 | (0.682, | 180 |
| | | | | 0.316) | |
| Example 90 | F152 | 4.12 | 38.9 | (0.681, | 299 |
| | | | | 0.318) | |
| Example 91 | F157 | 4.19 | 32.2 | (0.681, | 193 |
| | | | | 0.318) | |
| Example 92 | F158 | 4.22 | 37.1 | (0.681, | 280 |
| | | | | 0.318) | |
| Example 93 | F159 | 4.12 | 38.9 | (0.682, | 298 |
| | | | | 0.316) | |
| Example 94 | F170 | 4.12 | 39.6 | (0.681, | 368 |
| | | | | 0.318) | |
| Example 95 | F171 | 4.12 | 38.9 | (0.682, | 370 |
| | | | | 0.318) | |
| Comparative | A | 5.44 | 11.4 | (0.682, | 38 |
| Example 1 | | | | 0.318) | |
| Comparative | B | 5.29 | 14.2 | (0.682, | 52 |
| Example 2 | | | | 0.318) | |
| Comparative | C | 5.29 | 12.5 | (0.684, | 42 |
| Example 3 | | | | 0.316) | |
| Comparative | D | 4.92 | 26.2 | (0.683, | 21 |
| Example 4 | | | | 0.317) | |
| Comparative | E | 4.96 | 25.3 | (0.682, | 138 |
| Example 5 | | | | 0.318) | |
| Comparative | F | 4.88 | 20.9 | (0.683, | 121 |
| Example 6 | | | | 0.317) | |
| Comparative | G | 4.68 | 24.1 | (0.684, | 110 |
| Example 7 | | | | 0.316) | |
| Comparative | H | 4.79 | 21.9 | (0.683, | 62 |
| Example 8 | | | | 0.317) | |
| Comparative | I | 5.28 | 17.9 | (0.683, | 89 |
| Example 9 | | | | 0.317) | |

Looking at the results of Table 10 above, when the heterocyclic compound represented by Formula 1 is used as a light-emitting layer of the organic light-emitting device, it could be confirmed to exhibit an excellent effect in lifespan, luminous efficiency, and driving voltage properties compared to the case of using Comparative Compounds A to I.

In particular, it could be confirmed that the compound represented by Formula 1 improved hole injection and hole transport properties by substituting a cyclized fluorene group under the naphthocarbazole structure, and thus, the efficiency of an organic light-emitting device using an electron-blocking layer is effectively further improved.

In the present invention, it could be confirmed that when the compound represented by Formula 1 was used as a host for the light-emitting layer, excellent device properties were exhibited.

### DESCRIPTION OF SYMBOLS

100: Substrate
200: Anode
300: Organic layer
301: Hole injection layer
302: Hole transport layer
303: Light-emitting layer
304: Hole-blocking layer
305: Electron transport layer
306: Electron injection layer
400: Cathode

1. A heterocyclic compound represented by following Formula 1: 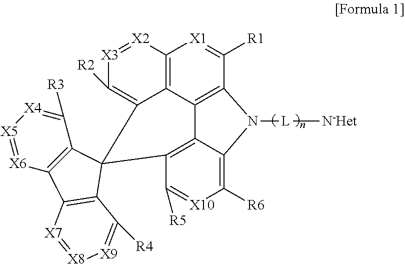
wherein,
X1 to X10 are the same as or different from each other and are each independently N or CRa;
Ra and R1 to R6 are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group; a substituted or unsubstituted C6 to C60 aryl group, or a substituted or unsubstituted C2 to C60 heteroaryl group,
L is a single bond; a substituted or unsubstituted C6 to C60 arylene group; or a substituted or unsubstituted C2 to C60 heteroarylene group,
n is an integer from 0 to 5, with the proviso that when n is 2 or more, L is the same as or different from each other,
N-Het is a substituted or unsubstituted and is C2 to C60 monocyclic or polycyclic heterocyclic group containing one or more N.

2. The heterocyclic compound according to claim 1, wherein Formula 1 is represented by any one of following Formulas 1-1 to 1-3: 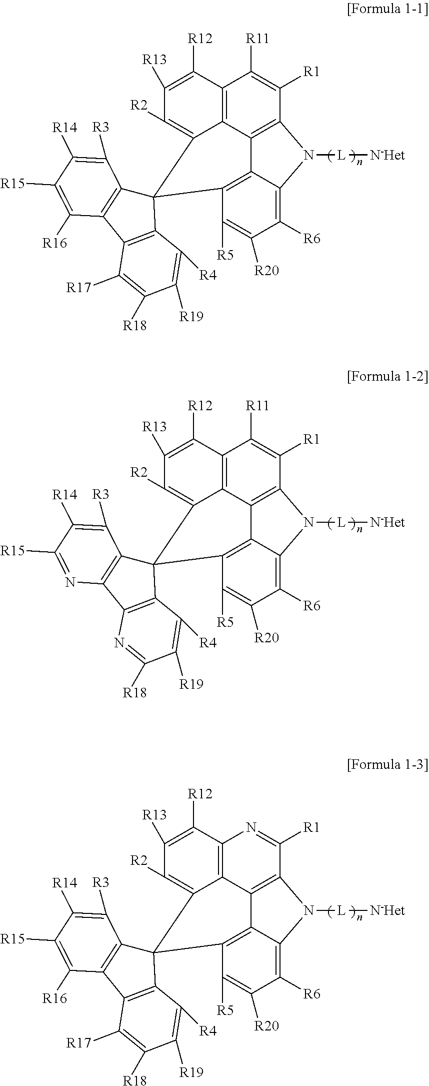
wherein,
R11 to R20 are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102, —SiR101R102R103, and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group; a substituted or unsubstituted C6 to C60 aryl group; or a substituted or unsubstituted C2 to C60 heteroaryl group,
R1 to R6, L, n, and N-Het are the same as defined in Formula 1.

3. The heterocyclic compound according to claim 1, wherein N-Het is represented by any one of following Formulas 1-4 to 1-8: 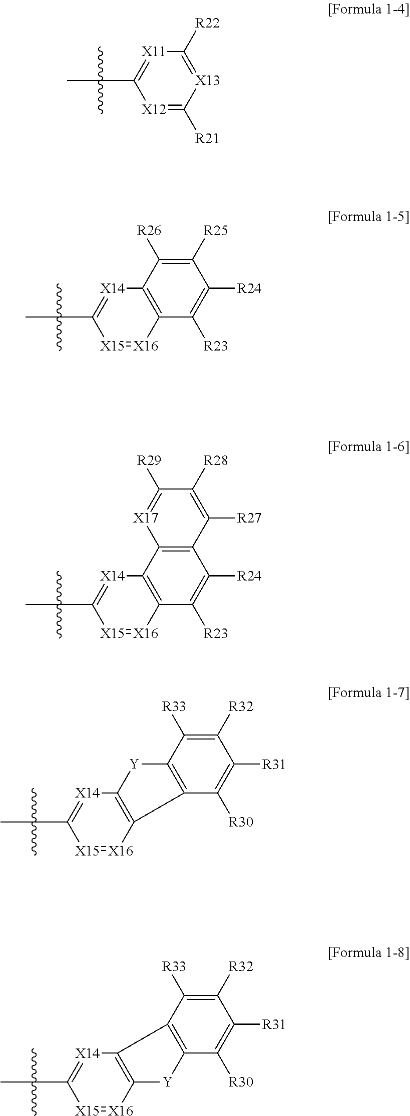
wherein,
X11 to X13 are the same as or different from each other and are each independently N or CRb,
X14 to X16 are the same as or different from each other and are each independently N or CRc,
X17 is N or CRd,
Rb, Rc, and Rd are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group; a substituted or unsubstituted C6 to C60 aryl group; or a substituted or unsubstituted C2 to C60 heteroaryl group,
Y is O or S,
R21 to R33 are the same as or different from each other and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocycle, wherein R101, R102, and R103 are the same as or different from each other and are each independently a substituted or unsubstituted C1 to C60 alkyl group; a substituted or unsubstituted C6 to C60 aryl group; or a substituted or unsubstituted C2 to C60 heteroaryl group.

4. The heterocyclic compound according to claim 2, wherein R1 to R6 and R11 to R20 are the same as or different from each other and are each independently hydrogen or deuterium.

5. The heterocyclic compound according to claim 2, wherein any one of R11 to R20 is a substituted or unsubstituted C6 to C60 aryl group or a substituted or unsubstituted C2 to C60 heteroaryl group, and the rest are hydrogen or deuterium.

6. The heterocyclic compound according to claim 2, wherein two of R11 to R20 are a substituted or unsubstituted C6 to C60 aryl group or a substituted or unsubstituted C2 to C60 heteroaryl group, and the rest are hydrogen or deuterium.

7. The heterocyclic compound according to claim 3, wherein two or more of X11 to X13 are N, and Rb is hydrogen; deuterium; a substituted or unsubstituted C6 to C60 aryl group; or a substituted or unsubstituted C2 to C60 heteroaryl group.

8. The heterocyclic compound according to claim 3, wherein two of X14 to X16 are N, and Rc is deuterium; a substituted or unsubstituted C6 to C60 aryl group; or a substituted or unsubstituted C2 to C60 heteroaryl group.

9. The heterocyclic compound according to claim 1, wherein Formula 1 is represented by any one of the following compounds:

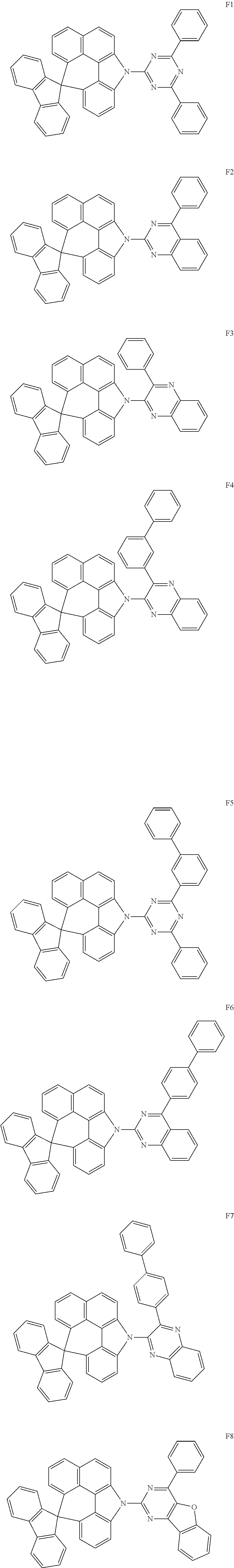

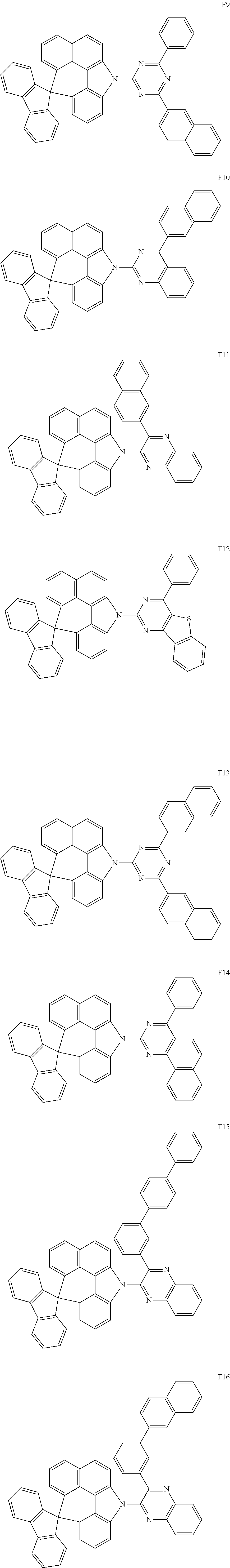

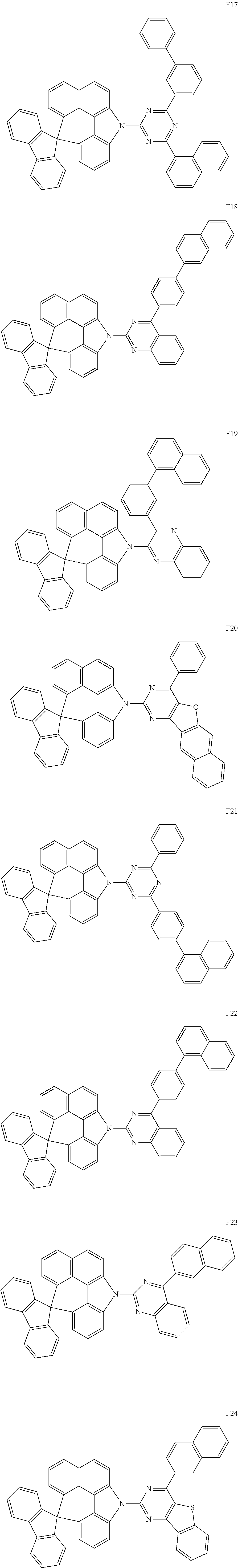

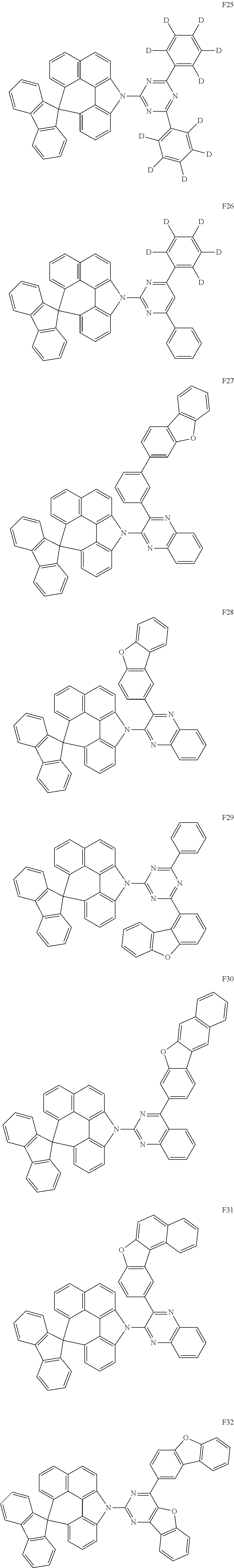

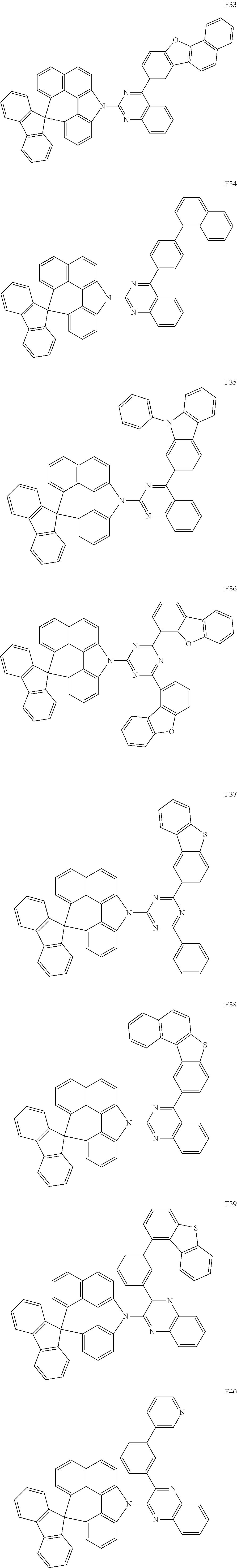

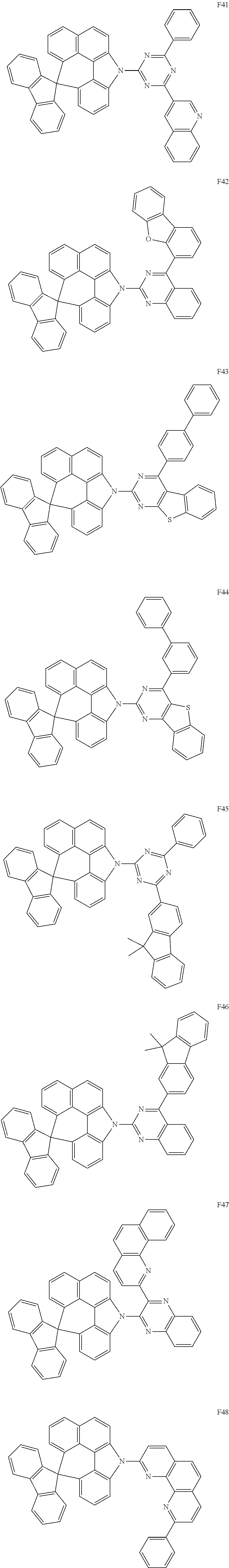

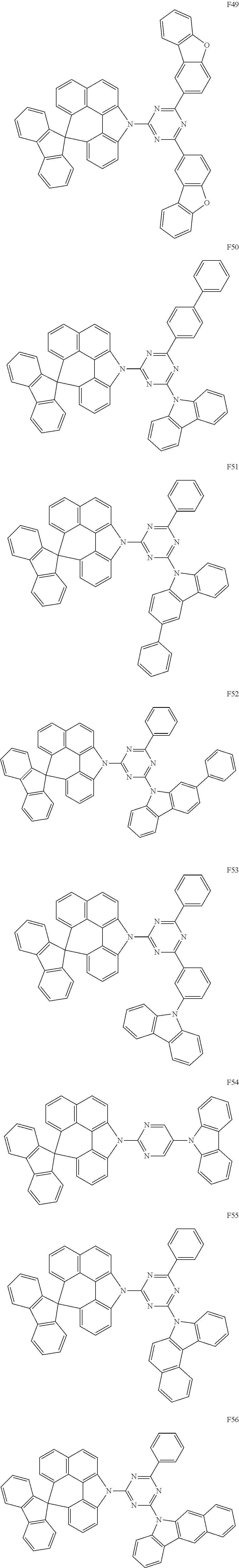

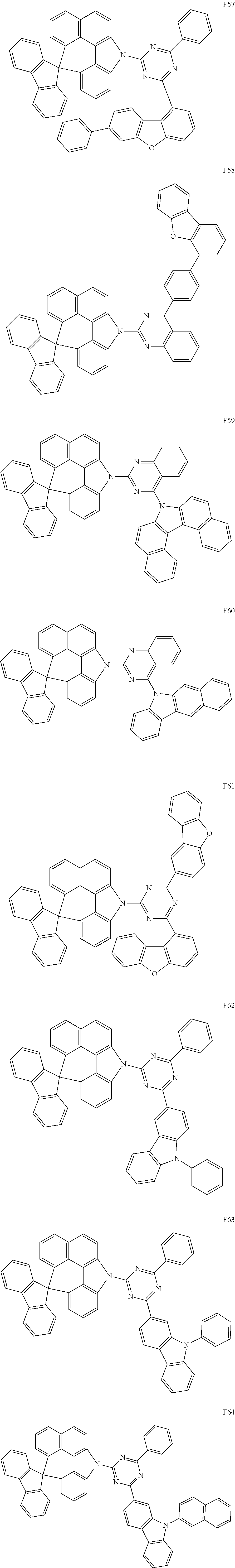

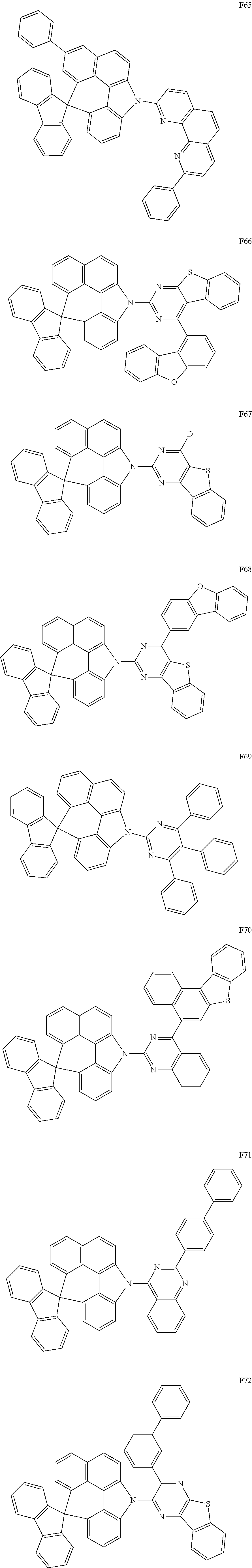

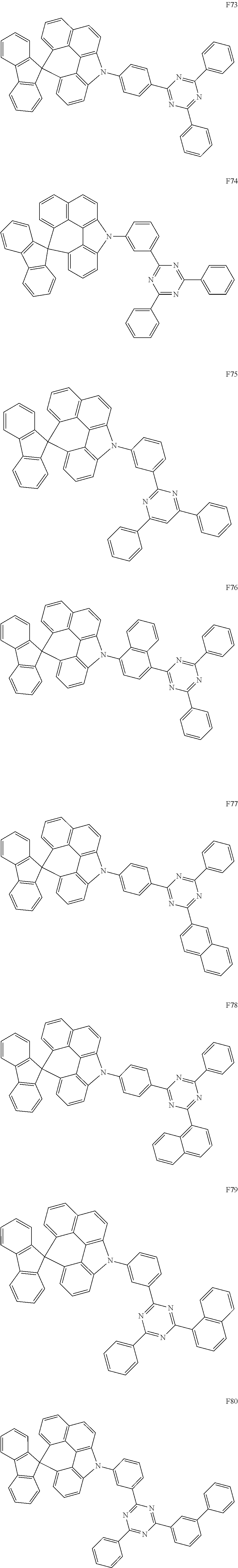

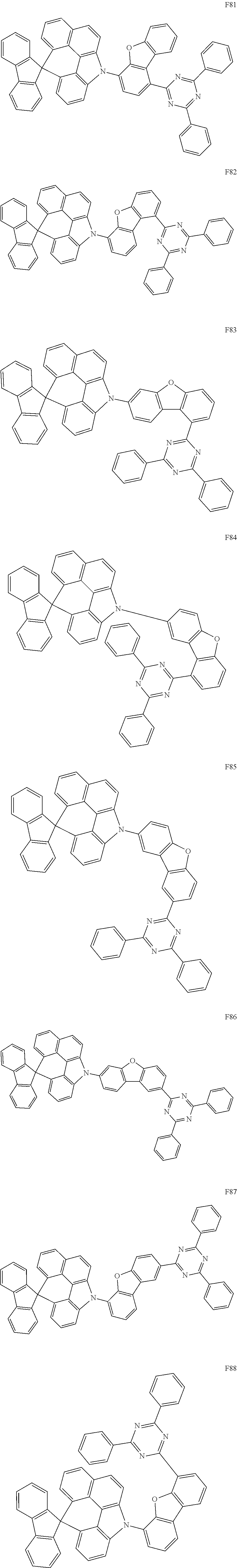

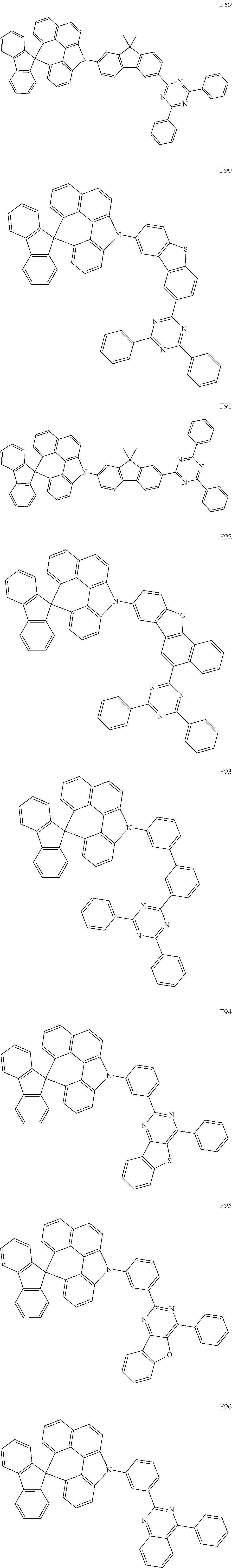

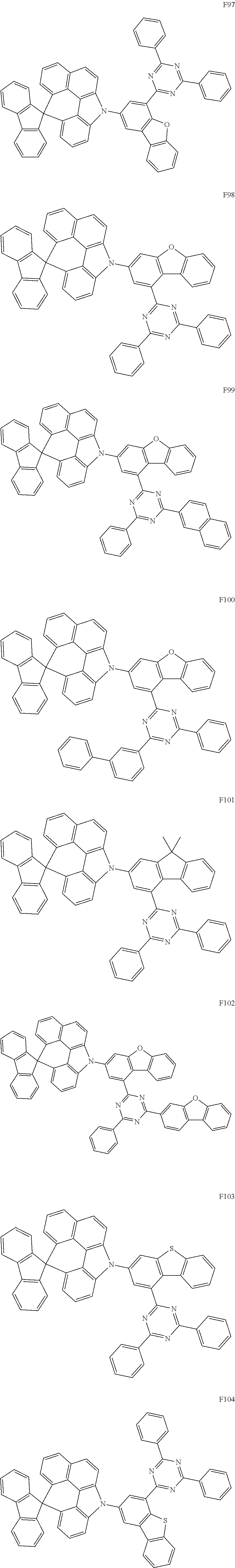

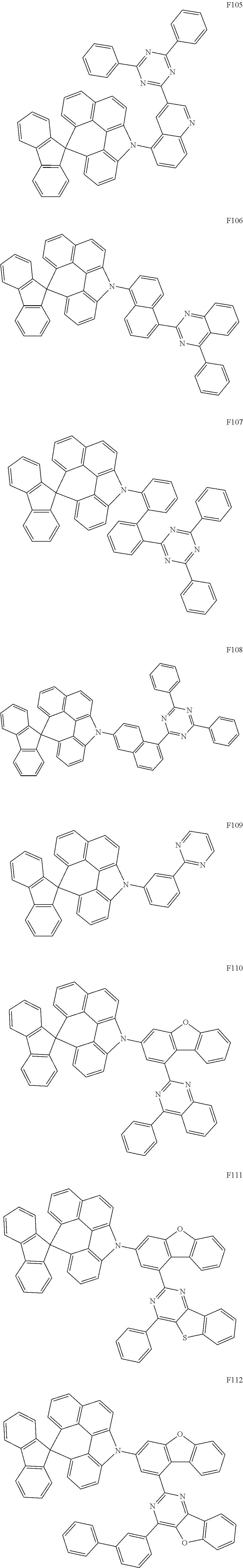

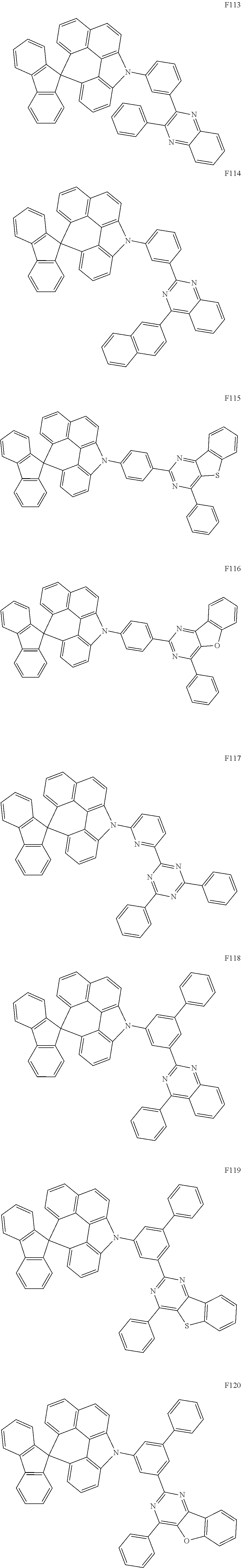

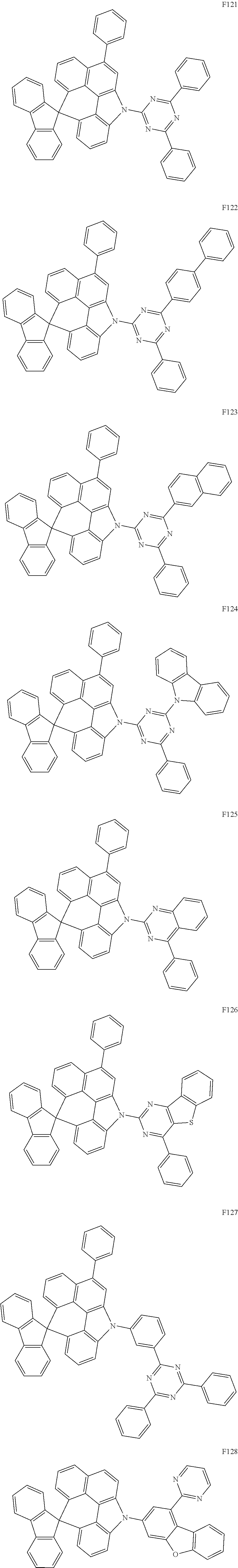

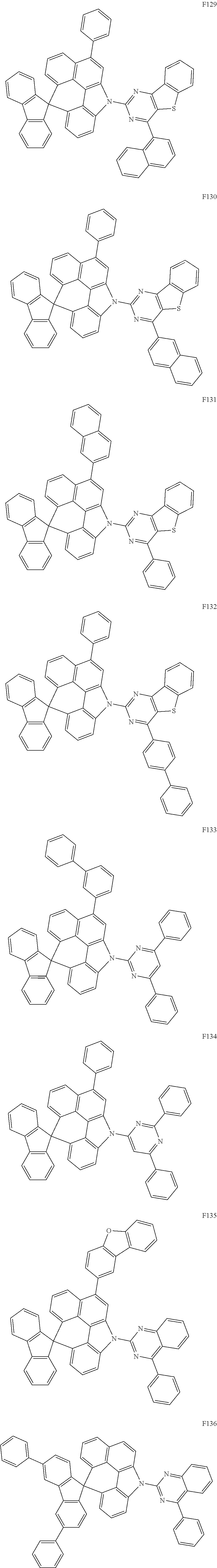

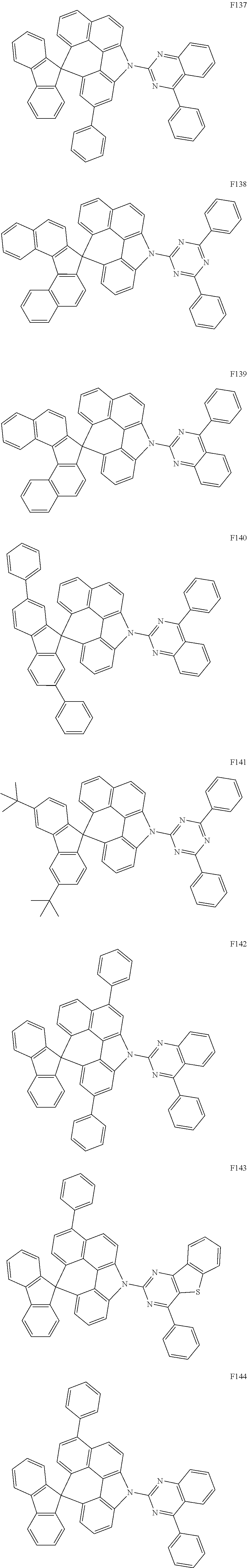

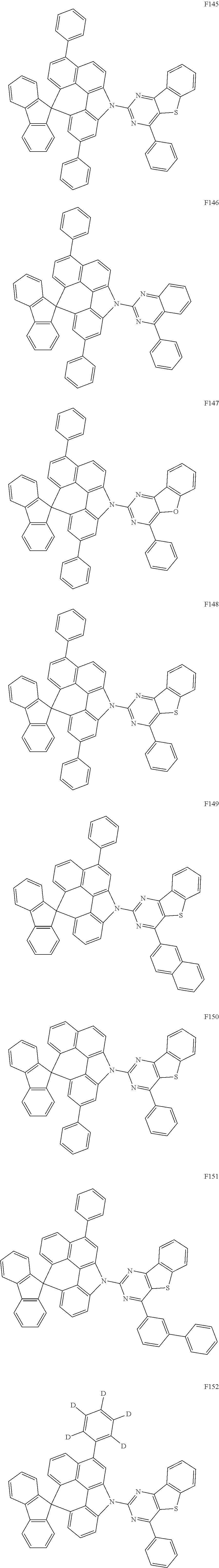

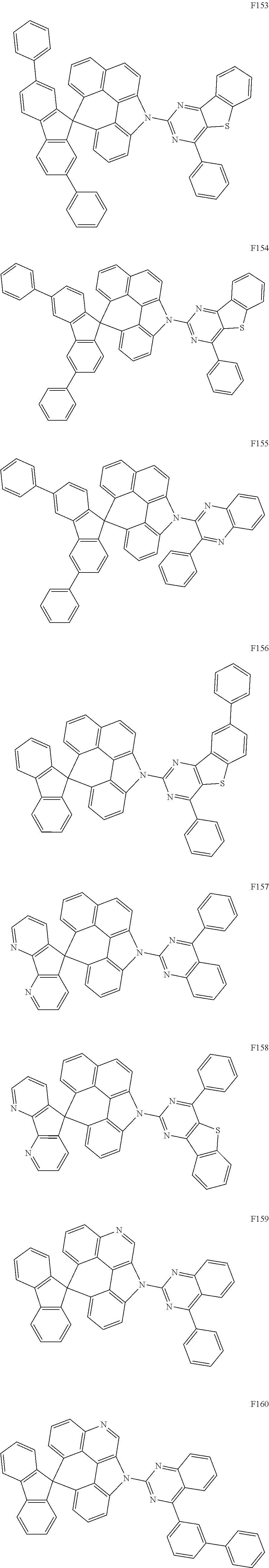

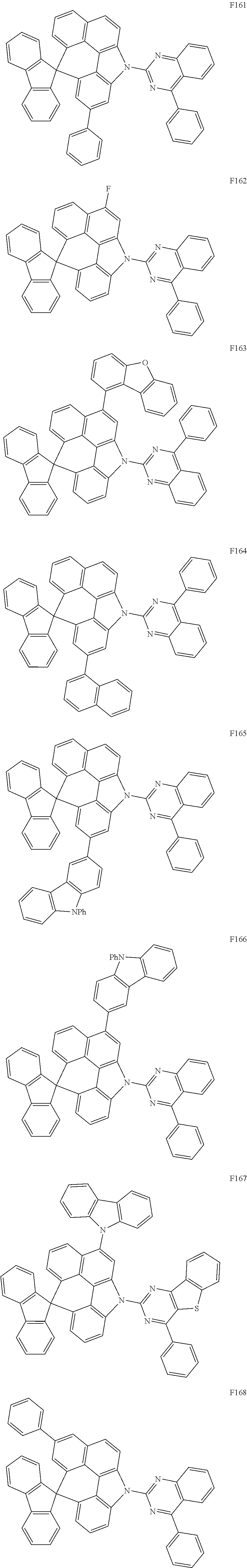

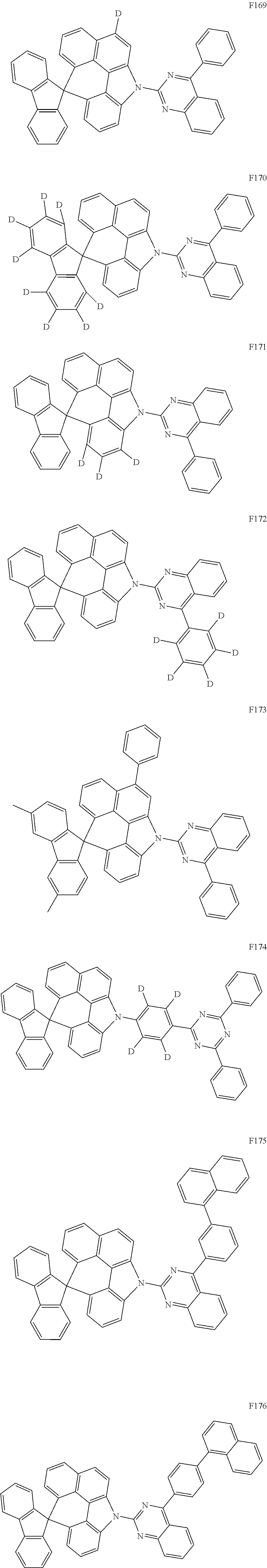

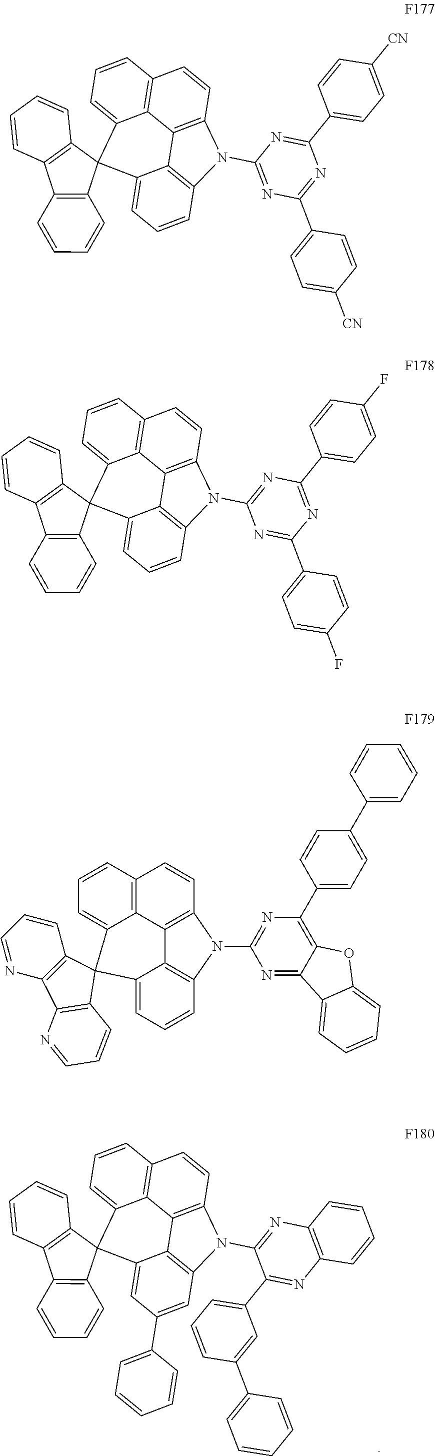 [SMILES: N#Cc1ccc(-c2nc(-c3ccc(C#N)cc3)nc(-n3c4cccc5c4c4c6c(cccc6ccc43)C53c4ccccc4-c4ccccc43)n2)cc1 ; Fc1ccc(-c2nc(-c3ccc(F)cc3)nc(-n3c4cccc5c4c4c6c(cccc6ccc43)C53c4ccccc4-c4ccccc43)n2)cc1 ; c1ccc(-c2ccc(-c3nc(-n4c5cccc6c5c5c7c(cccc7ccc54)C64c5cccnc5-c5ncccc54)nc4c3oc3ccccc34)cc2)cc1 ; c1ccc(-c2cccc(-c3nc4ccccc4nc3-n3c4cc(-c5ccccc5)cc5c4c4c6c(cccc6ccc43)C53c4ccccc4-c4ccccc43)c2)cc1]

10. An organic light-emitting device comprising:
a first electrode;
a second electrode provided to face the first electrode; and
one or more organic layers provided between the first electrode and the second electrode, and
wherein at least one of the one or more organic layers comprise the heterocyclic compound according to claim 1.

11. The organic light-emitting device according to claim 10, wherein the organic layer comprises a light-emitting layer, the light-emitting layer comprises a host material, and the host material comprises the heterocyclic compound.

12. The organic light-emitting device according to claim 10, wherein the organic light-emitting device further comprises one or more layers selected from the group consisting of a light-emitting layer, a hole injection layer, a hole transport layer, an electron injection layer, an electron transport layer, an electron-blocking layer, and a hole-blocking layer.

13. A composition for an organic layer of an organic light-emitting device, comprising the heterocyclic compound according to claim 1.

14. (canceled)
